SEQUENCE LISTING (8 sequences)

SEQ ID NO: 1 - DNA, 21 bp, Artificial sequence
  Synthetic
caagcugacccugaaguuctt

SEQ ID NO: 2 - RNA, 20 nt, Artificial sequence
  Synthetic
aacuucagggucagcuugcc

SEQ ID NO: 3 - DNA, 21 bp, Artificial sequence
  Synthetic
aguacugcuuacgauacggtt

SEQ ID NO: 4 - DNA, 21 bp, Artificial sequence
  Synthetic
ccguaucguaagcaguacutt

SEQ ID NO: 5 - RNA, 21 nt, Artificial sequence
  Synthetic
agaaggagaucguggacuauu

SEQ ID NO: 6 - RNA, 21 nt, Artificial sequence
  Synthetic
uaguccacgaucuccuucuuu

SEQ ID NO: 7 - DNA, 21 bp, Artificial sequence
  Synthetic
gcaugcgaccucuguuugatt

SEQ ID NO: 8 - DNA, 21 bp, Artificial sequence
  Synthetic
ucaaacagaggucgcaugctt

(19) United States
(12) Patent Application Publication
(10) Pub. No.: US 2025/0009675 A1
(43) Pub. Date: Jan. 9, 2025

(54) PHOTO-CROSSLINKED BIOREDUCIBLE POLYMERIC NANOPARTICLES FOR ENHANCED RNA DELIVERY

(71) Applicant: The Johns Hopkins University, Baltimore, MD (US)

(72) Inventors: Jordan J. Green, Woodstock, MD (US); Johan Karlsson, Baltimore, MD (US); Stephany Yi Tzeng, Somerville, MD (US); Kathryn M. Luly, Baltimore, MD (US); Yuan Rui, Baltimore, MD (US); David Wilson, Smithsburg, MD (US); Kristen Kozielski, Baltimore, MD (US)

(21) Appl. No.: 18/274,133

(22) PCT Filed: Jan. 25, 2022
(86) PCT No.: PCT/US2022/013604

§ 371 Date: Jul. 25, 2023

Related U.S. Application Data
(60) Provisional application No. 63/141,242, filed on Jan. 25, 2021.

(51) Int. Cl. A61K 9/51; B82Y 5/00; C12N 15/113

(52) U.S. Cl. CPC A61K 9/5153; C12N 15/113; B82Y 5/00; C12N 2310/14

(57) ABSTRACT

Nanocarriers are engineered to realize the potential of RNA therapeutics. This work reports the design of pho-to-crosslinked bioreducible nanoparticles (XbNPs) for stable siRNA encapsulation in high serum conditions, shielded surface charge, efficient intracellular trafficking, and triggered cytosolic RNA release. These attributes of XbNPs leads to robust siRNA-mediated knockdown in cancer cells and potent systemic siRNA delivery to tumors in the lungs.

### STATEMENT OF GOVERNMENTAL INTEREST

This invention was made with government support under CA195503, EB028239, CA246699, and CA228133 awarded by the National Institutes of Health. The government has certain rights in the invention.

### SEQUENCE LISTING

The text of the computer readable sequence listing filed herewith, titled “39216-601_SEQUENCE_LISTING_ST25”, created Jan. 25, 2022, having a file size of 1,709 bytes, is hereby incorporated by reference in its entirety.

### BACKGROUND

Delivery of exogenous nucleic acids to precisely modulate gene expression in specific cells is recognized to have tremendous potential in the treatment of a wide-range of human diseases. Yin et al., 2014. One such technology is RNA interference (RNAi) using small interfering RNA (siRNA) with the ability to cause sequence-specific gene silencing of almost any sequence in the genome upon introduction into a cell. Despite this potential, clinical applications of this technology have been limited due to inefficient siRNA delivery across biological barriers. Whitehead et al., 2009. Delivery technologies using chemically modified siRNA molecules and viral vectors have yet to overcome several limitations, including immunogenicity, limited payload capacity, difficulty of scaled-up vector production, and inefficient silencing. Bessis et al., 2004; Thomas et al., 2003.

In contrast, non-viral nanoparticle-based siRNA delivery formulations have the potential to resolve these major issues as they are generally less immunogenic and easier to manufacture, and enable greater siRNA loading. Pack et al., 2005; Mintzer and Simanek, 2009. Recently, the first RNAi technology received regulatory approval using a lipid nanoparticle-based formulation for siRNA delivery for the treatment of polyneuropathies induced by hereditary transthyretin amyloidosis. Adams et al., 2018; Akinc et al., 2019. This landmark approval will undoubtedly pave the way for future RNAi nanomedicines and has introduced a new paradigm for genetic medicine.

Nanocarriers for cytosolic delivery of siRNA therapeutics must overcome several challenges, however, for successful knockdown, including efficient cargo encapsulation, cellular uptake by targeted cells, endosomal escape, and timely cytosolic release. In addition, the bioavailability of many nanoparticle systems is often limited to the liver following systemic administration; thus, there is a need for nanomaterials that can enable tissue-mediated extrahepatic delivery, as well as for nanomaterials that are biodegradable and non-toxic. Longmire et al., 2008.

### SUMMARY

In some aspects, the presently disclosed subject matter provides a nanoparticle comprising a bioreducible cationic polymer, a crosslinking polymer and one or more nucleic acids, wherein the bioreducible cationic polymer and crosslinking polymer are crosslinked and the one or more nucleic acids are encapsulated within the nanoparticle.

In some aspects, the bioreducible cationic polymer comprises a bioreducible poly (beta-amino ester). In certain aspects, the bioreducible poly(beta-amino ester) comprises at least one degradable ester bond and at least one disulfide bond. In particular aspects, the bioreducible poly(beta-amino ester) comprises an amine-terminated bioreducible poly(beta-amino ester).

In some aspects, the amine-terminated bioreducible poly(beta-amino ester) comprises a monomer having the following structure:

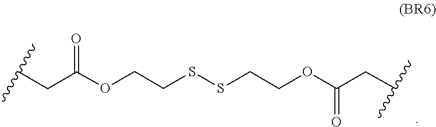 [SMILES: *CC(=O)OCCSSCCOC(=O)C*]

In some aspects, the amine-terminated bioreducible poly(beta-amino ester) comprises a compound having the following structure:

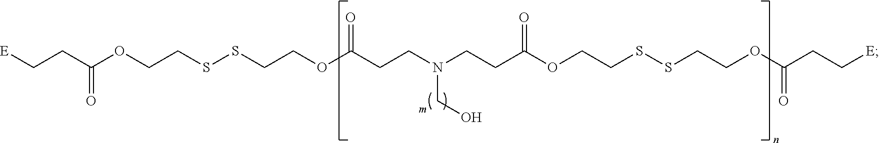

wherein:
n is an integer from 1 to 10,000;
m is an integer selected from the group consisting of 2, 3, 4, 5, 6, 7, 8, and 9; and
E is an amine-containing endcapping group.

In certain aspects, the amine-containing endcapping group is selected from the group consisting of:

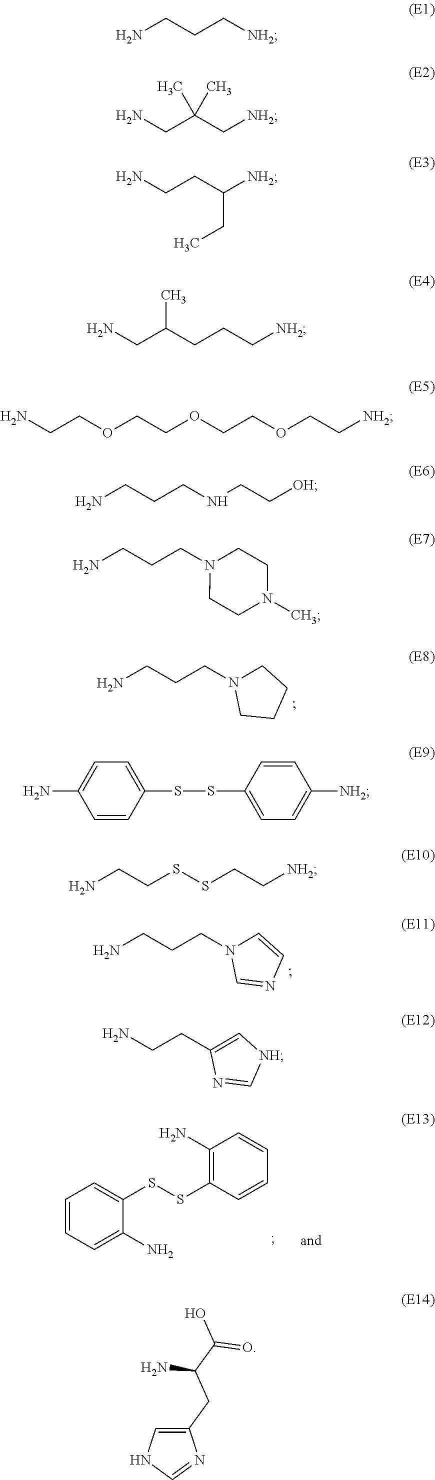

In some aspects, the endcapping groups include:

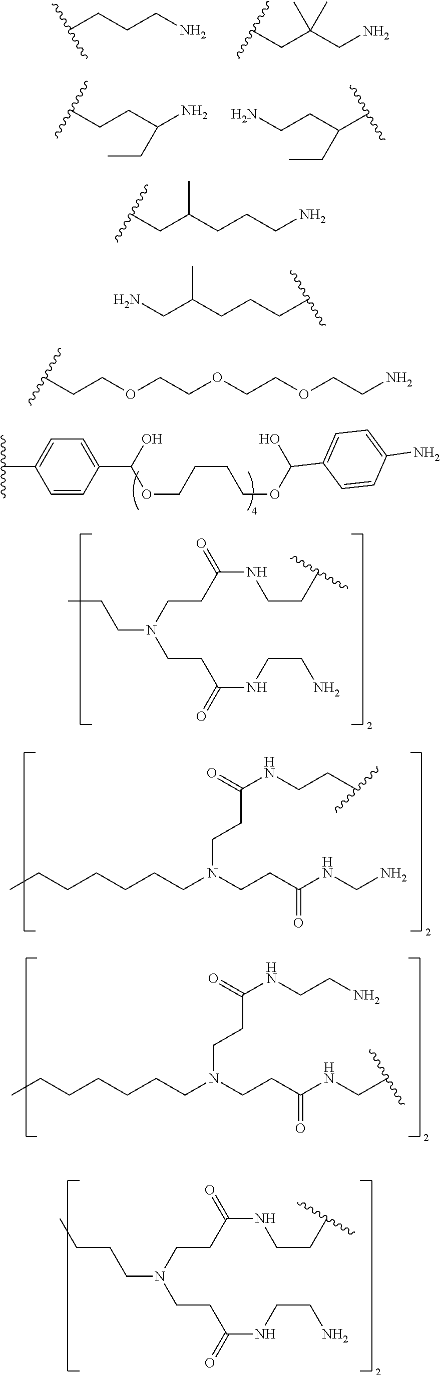

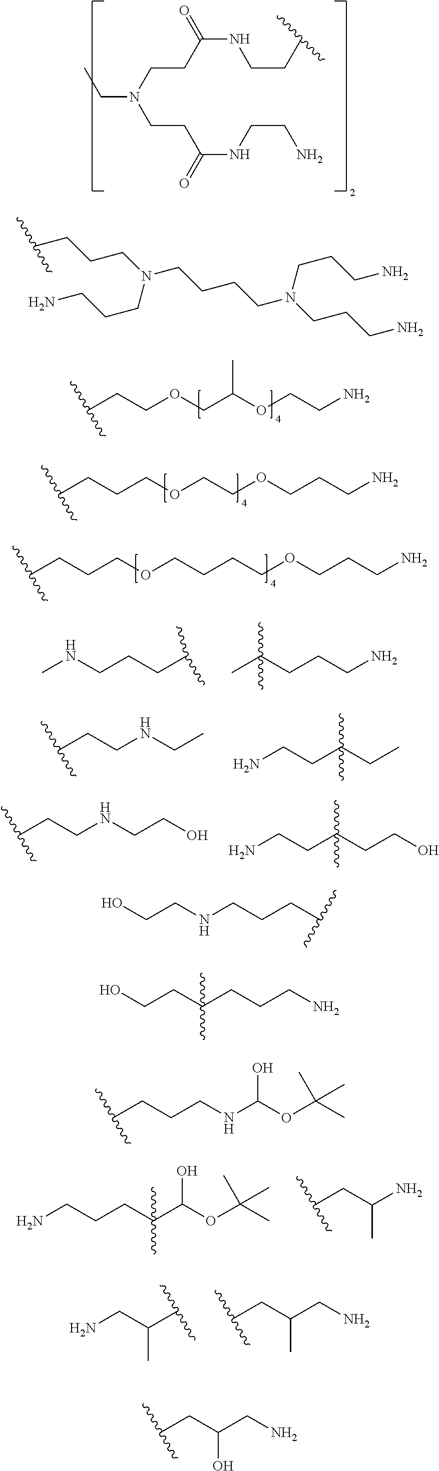

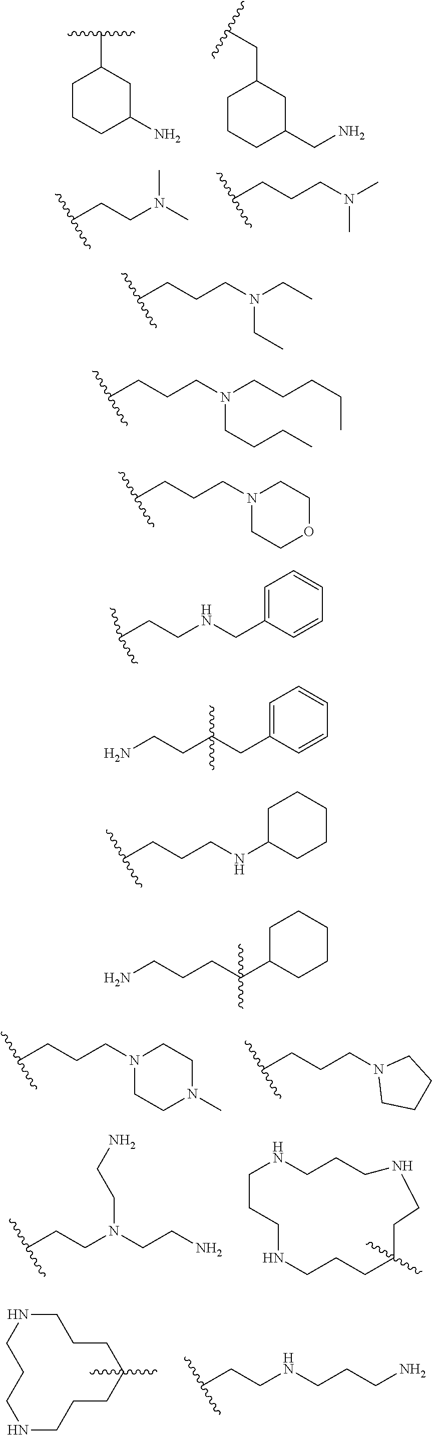

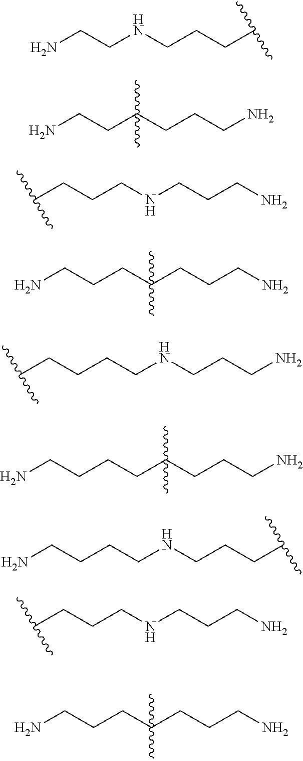

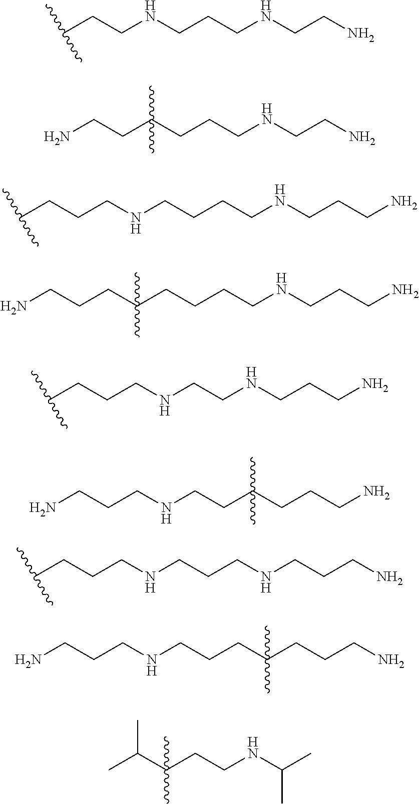

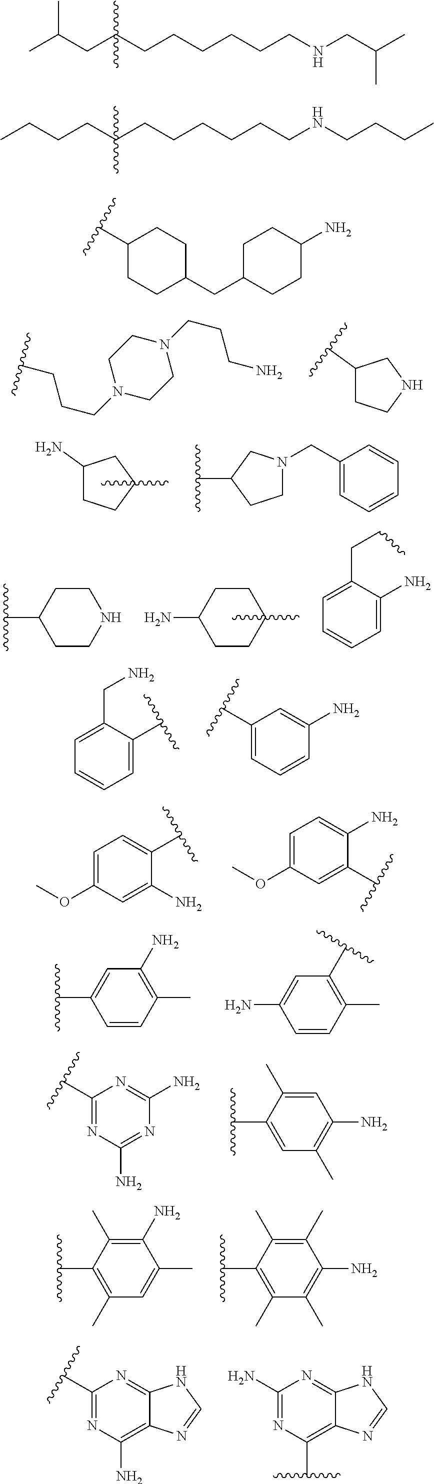

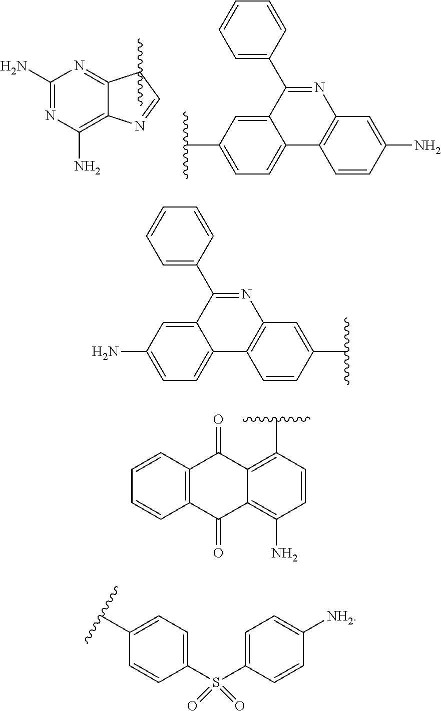

In other aspects, the endcapping groups include:

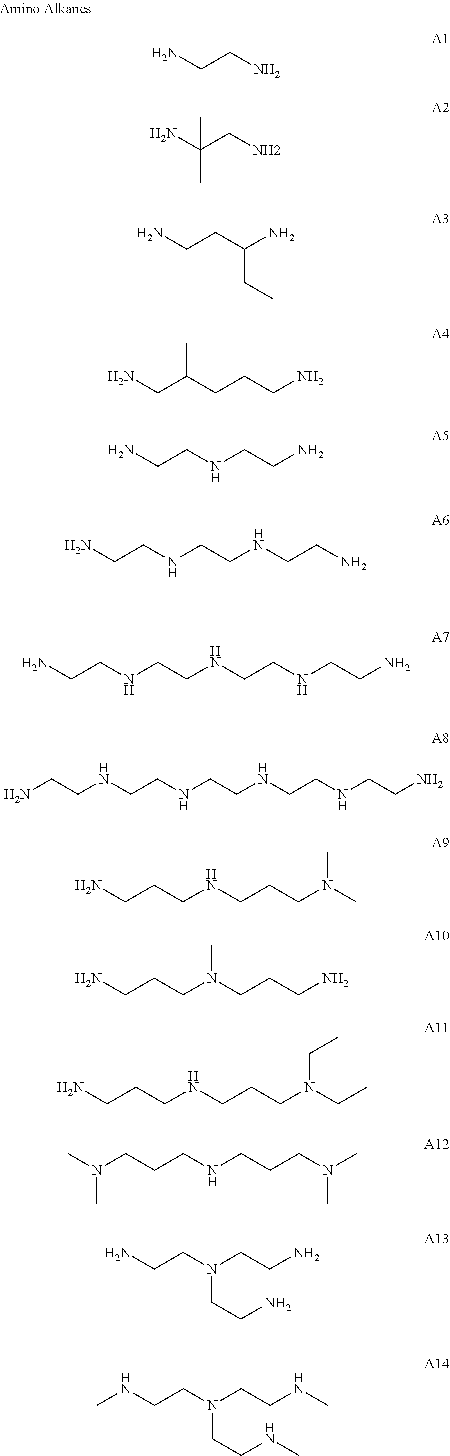

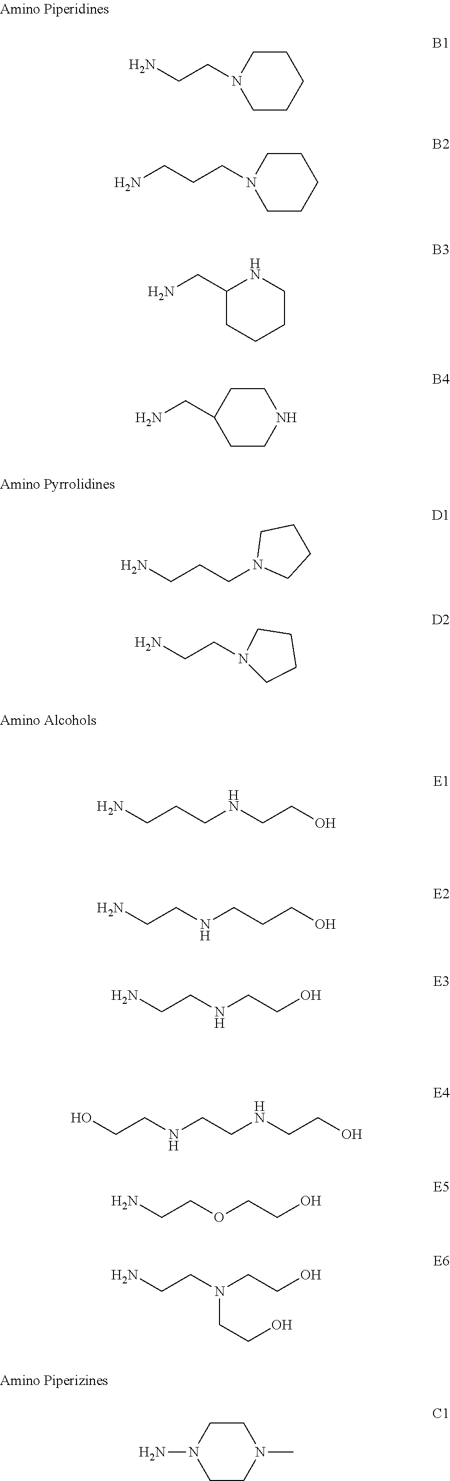

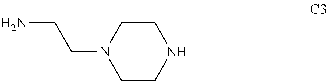

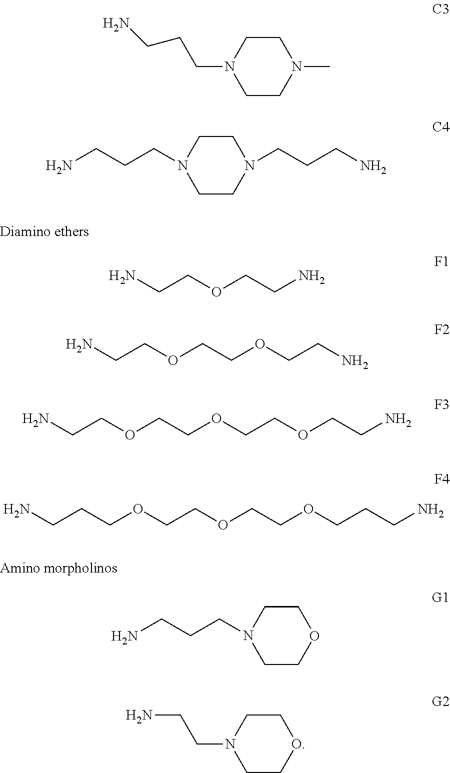

In yet other aspects, the endcapping groups include:

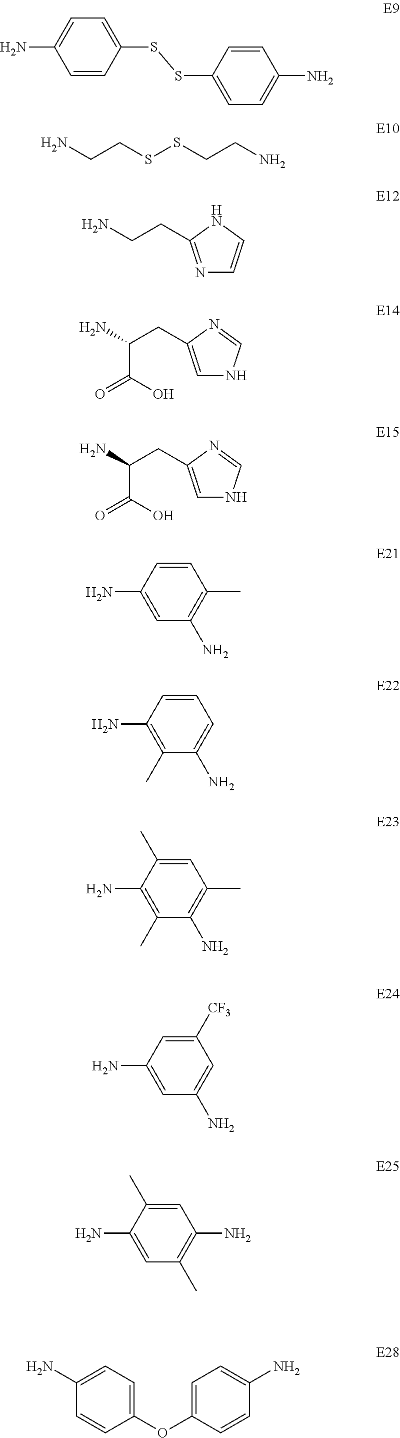

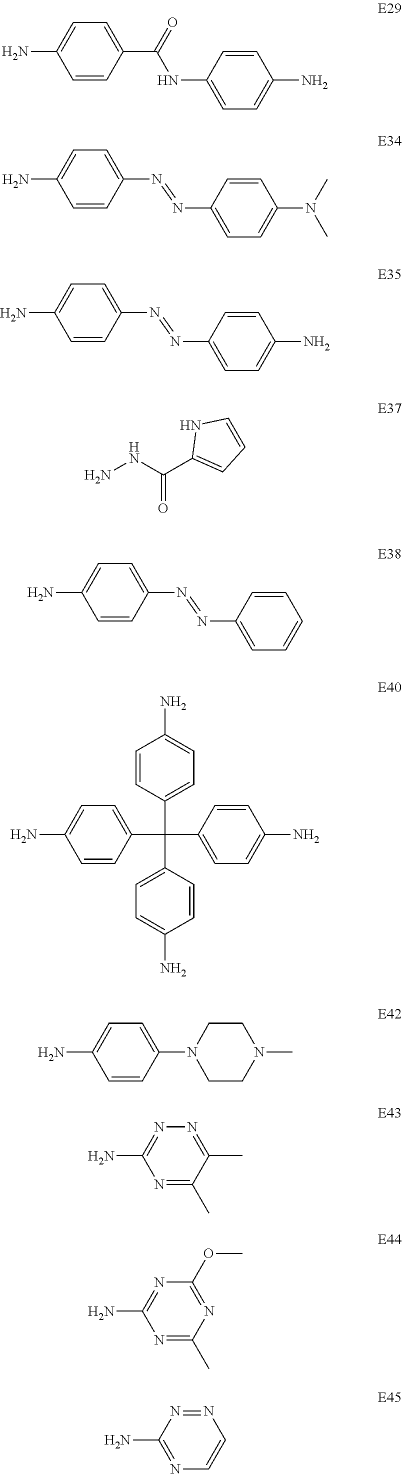

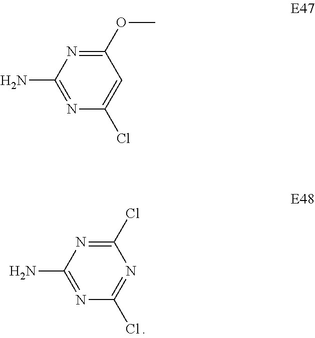

In particular aspects, the amine-terminated bioreducible poly(beta-amino ester) is selected from the group consisting of R646 and R647:

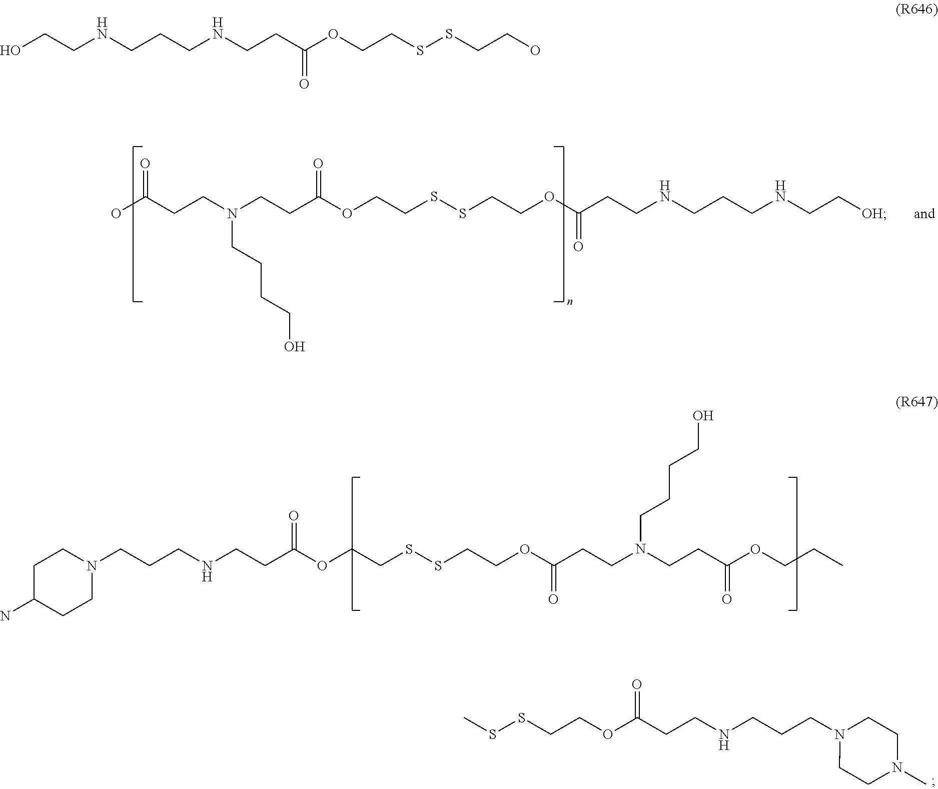

wherein n is an integer from 1 to 10,000.

In some aspects, the crosslinking polymer comprises an acrylate-terminated bioreducible poly(beta-amino ester) having at least one disulfide bond. In certain aspects, the acrylate-terminated bioreducible poly(beta-amino ester) comprises a monomer having the following structure:

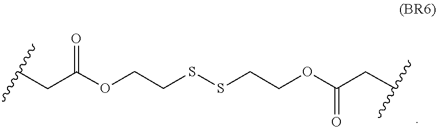 [SMILES: *CC(=O)OCCSSCCOC(=O)C*]

In particular aspects, the acrylate-terminated bioreducible poly(beta-amino ester) comprises a compound having the following structure:

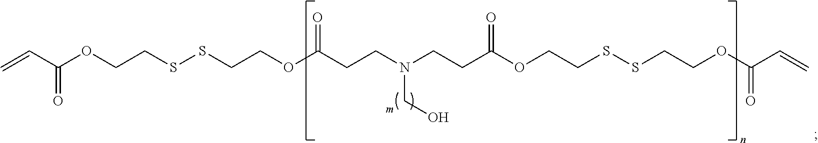

wherein:
n is an integer from 1 to 10,000; and
m is an integer selected from the group consisting of 2, 3, 4, 5, 6, 7, 8, and 9.

In yet more particular aspects, the acrylate-terminated bioreducible poly(beta-amino ester) comprises R64-Ac:

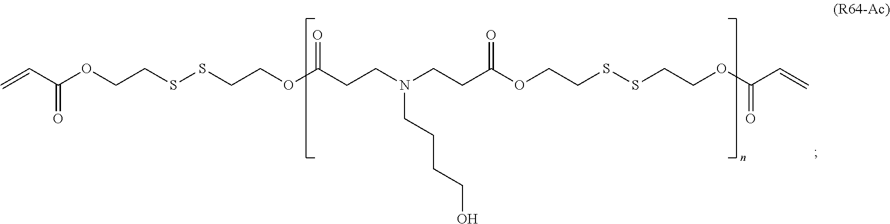

wherein n is an integer from 1 to 10,000.

In some aspects, the crosslinking polymer and the cationic polymer have a mass ratio (weight ratio) of about 1:1 to 1:5 (w/w). In certain aspects, the crosslinking polymer and the cationic polymer have a mass ratio (weight ratio) of about 1:3 (w/w).

In some aspects, the one or more nucleic acids are selected from the group consisting of DNA, RNA, siRNA, mRNA, miRNA, isRNA, agRNA, and smRNA. In certain aspects, the one or more nucleic acids comprises siRNA.

In some aspects, the nanoparticle has a polymer:nucleic acid mass ratio (weight/weight or w/w) ranging from about 20 (w/w) polymer:nucleic acid to about 2,000 (w/w) polymer:nucleic acid.

In some aspects, the nanoparticle has a substantially neutral surface charge.

In some aspects, the nanoparticle has a particle size ranging from about 50 nm to 500 nm. In certain aspects, the nanoparticle has a particle size ranging from about 100 nm to 250 nm.

In other aspects, the presently disclosed subject matter provides a pharmaceutical formulation comprising the presently disclosed nanoparticle and a pharmaceutically acceptable carrier.

In yet other aspects, the presently disclosed subject matter provides a method for preparing a crosslinked nanoparticle, the method comprising:
(a) mixing a bioreducible cationic polymer, and crosslinking polymer, and one or more nucleic acids to form a self-assembled bioreducible nanoparticle;
(b) incubating the self-assembled bioreducible nanoparticle for a period of time;
(c) adding a photoinitiator to the self-assembled bioreducible nanoparticle; and
(d) exposing the self-assembled bioreducible nanoparticle and photoinitiator to ultraviolet light to form a crosslinked bioreducible nanoparticle comprising the one or more nucleic acids.

In yet other aspects, the presently disclosed subject matter provides a method for treating a cancer, the method comprising administering to a subject in need of treatment thereof a therapeutically effective amount of the presently disclosed nanoparticle or a pharmaceutical formulation thereof to treat the cancer.

In some aspects, the nanoparticle enters a cytosol of a cell of the subject. In some aspects, the at least one disulfide bond of the bioreducible poly(beta-amino ester) is reductively degraded in the cytosol to release the nucleic acid in the cytosol of the cell of the subject. In some aspects, the at least one disulfide bond is reductively degraded via glutathione.

In some aspects, the method comprises systemically administering the nanoparticle or pharmaceutical formulation. In some aspects, the one or more nanoparticles are delivered to one or more organs beyond the liver. In certain aspects, the one or more organs beyond the liver comprises the lungs.

In some aspects, the method comprises preferential uptake of the nucleic acid in one or more cancer cells. In some aspects, the crosslinked nanoparticle exhibits enhanced colloidal stability in the bloodstream compared to a non-crosslinked nanoparticle. In some aspects, the crosslinked nanoparticle exhibits reduced adsorption of anionic serum proteins compared to a non-crosslinked nanoparticle.

In some aspects, the cancer is selected from the group consisting of brain cancer, melanoma, lung cancer, breast cancer, prostate cancer, and colorectal cancer. In certain aspects, the brain cancer comprises a glioblastoma.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one figure executed in color. Copies of this patent or patent application publication with color figures will be provided by the Office upon request and payment of the necessary fee.

Having thus described the presently disclosed subject matter in general terms, reference will now be made to the accompanying Figures, which are not necessarily drawn to scale, and wherein:

FIG. 1A, FIG. 1B, and FIG. 1C demonstrate photo-crosslinked bioreducible nanoparticles (XbNPs) for siRNA delivery. (FIG. 1A) Schematic illustration of the electrostatic-based self-assembly into nanoparticles (NPs) and subsequent photo-crosslinking. (FIG. 1B) Reaction scheme of Michael Addition used to form the bioreducible polymers. In the initial step, the diacrylate backbone monomer BR6 is polymerized with the side chain monomer S4 forming the acrylate terminated crosslinking polymer R64-Ac. To form the amine-terminated polymer, a second synthesis step was used, in which the base polymer R64-Ac was endcapped by either monomer E6 or E7 to form R646 and R647, respectively. (FIG. 1C) Molecular weight of polymeric nanocarrier assessed by GPC for crosslinked (Xlinked) and non-crosslinked (Non-Xlinked) NPs with and without exposure to UV light. The NP formulations were formed at a polymer/siRNA ratio of 900 w/w;

FIG. 2A, FIG. 2B, FIG. 2C, FIG. 2D, FIG. 2E, FIG. 2F, FIG. 2G, FIG. 2H, and FIG. 2I demonstrate that photo-crosslinking reduced the nanoparticle (NP) surface charge of the otherwise cationic nanocarrier. (FIG. 2A) Nanoparticle hydrodynamic diameter assessment by dynamic light scattering (DLS) for with (Xlinked) and without (Non-Xlinked) crosslinking when incubated in PBS containing 0%, 10%, and 50% serum. (FIG. 2B) Nanoparticle tracking analysis (NTA) of nanoparticle hydrodynamic diameter. (FIG. 2C) Representative TEM images of Non-Xlinked (left) and Xlinked (right) NPs. Long-term NP stability assessed with DLS when incubated in (FIG. 2D) 10% and (FIG. 2E) 50% serum over 4 h. (FIG. 2F) The hydrodynamic diameter of NP formulations using different polymer/siRNA ratios (w/w). (FIG. 2G) Zeta potential measurements demonstrated reduced surface charge of nanoparticles after crosslinking (*:p<0.0001; n=3). (FIG. 2H) Surface charge of nanoparticles with (XL) and without (Non-XL) crosslinking incubated in 50% serum. The zeta potential for Non-XL NPs was statistically lower compared to 50% serum by itself (*:p=0.0039; n=3). (FIG. 2I) Surface charge of XbNPs formulations using different polymer/siRNA ratios (w/w). One-way ANOVA followed by Tukey's post hoc test were used for statistical analyses. Error bar represents standard error of mean (SEM);

FIG. 3A, FIG. 3B, FIG. 3C, and FIG. 3D demonstrate that photo-crosslinked nanoparticles (XbNPs) lowered protein adsorption when incubated in serum and improved siRNA encapsulation efficiency in a high serum condition. (FIG. 3A) The protein adsorption was assessed by the bicinchoninic acid (BCA) assay for crosslinked (Xlinked) and non-crosslinked (Non-Xlinked) NPs using formulations of 1200 and 900 w/w ratios (n=3). *:p<0.0001, as determined by two-way ANOVA followed by Sidak's multiple comparisons. Error bars represents standard error of mean (SEM). (FIG. 3B) Sodium dodecyl sulfate-polyacrylamide gel electrophoresis (SDS-PAGE) of adsorbed proteins following incubation in serum or PBS for nanoparticles with (XL) and without (Non-XL) crosslinking. Gel electrophoresis assessment of (FIG. 3C) siRNA-encapsulation efficiency for crosslinked (Xlinked) and non-crosslinked (Non-Xlinked) NPs using formulations of 1200, 900, 300, and 200 w/w ratios when incubated in 50% serum for 4 h, and (FIG. 3D) siRNA release when incubated in cytosolic mimicking environment of 10×10⁻³ M Glutathione (GSH);

FIG. 4A, FIG. 4B, FIG. 4C, FIG. 4D, FIG. 4E, FIG. 4F, FIG. 4G, FIG. 4H, FIG. 4I, and FIG. 4J show that XbNPs provided superior siRNA-mediated knockdown in patient-derived glioblastoma cells (GBM319) in high serum conditions and following pre-incubation for 6 h compared to non-crosslinked nanoparticles (NPs). In vitro transfection in 50% serum of GBM319-GFP⁺ cells assessed by flow cytometry analyzing siRNA-mediated knockdown for (FIG. 4A) crosslinked (Xlinked) and non-crosslinked (Non-Xlinked) nanoparticles formulated with different ratio of acrylate-terminated (Ac) and amine-endcapped (E) polymers (*:p<0.0001; two-way ANOVA followed by Sidak's multiple comparisons), (FIG. 4B) Xlinked NPs prepared with different UV exposure times (*:p<0.0001; one-way ANOVA). (FIG. 4C) Representative fluorescence microscopy images of GBM319-GFP⁺ treated with XbNPs carrying either scRNA or siRNA targeting GFP (scale bars=200 μm). (FIG. 4D) siRNA-mediated knockdown (*:p<0.05; two-way ANOVA followed by Sidak's multiple comparisons) and (FIG. 4E) viability (*:p<0.005; two-way ANOVA followed by Sidak's multiple comparisons) assessed by MTS assay for transfection in 50% serum of Non-Xlinked and Xlinked NPs formulated with either R646 or R647 as the amine-terminated polymer using 900 or 1200 w/w formulations. siRNA-mediated knockdown of XbNPs with altered (FIG. 4F) polymer (*:p<0.0001; one-way ANOVA followed by Tukey's post hoc test) and (FIG. 4G) siRNA (*:p<0.01; one-way ANOVA followed by Tukey's post hoc test) concentrations. (FIG. 4H) siRNA-mediated knockdown and (FIG. 4I) viability in GBM319 when transfected with XbNPs in 50% and 100% serum. (FIG. 4J) Transfection following pre-incubation at varied times in complete serum (*:p<0.0001; two-way ANOVA followed by Sidak's multiple comparisons). Error bars represent standard error of mean (SEM) and n=4;

FIG. 5A, FIG. 5B, FIG. 5C, FIG. 5D, FIG. 5E, and FIG. 5F demonstrate that XbNPs enhanced cellular uptake compared to Non-Xlinked NPs in patient-derived glioblastoma cells (GBM319) upon transfection in high serum conditions. Cellular uptake in GBM319 cells assessed by flow cytometry after (FIG. 5A) 6 h (*:p<0.0001; n=4) and (FIG. 5B) 24 h (*:p<0.0001; n=4) following transfection in 50% and 100% serum for crosslinked (Xlinked) and non-crosslinked (Non-Xlinked) nanoparticles (NPs) carrying Cy5-labeled siRNA. (FIG. 5C) Confocal microscopy images of cellular uptake for nanoparticle (NP) carrying Cy5-labeled siRNA 24 and 48 h post-treatment (scale bars=20 μm). (FIG. 5D) Representative image 24 h post-treatment with XbNPs showing nanoparticle (Cy5) and lysosome/endosome colocalization in yellow. (FIG. 5E) Representative 2D scattergram 24 h post-treatment with XbNPs, in which region 3 represents colocalized pixel intensities. (FIG. 5F) NP and lysosome/endosome colocalization 24 and 48 h post-treatment with Xlinked and Non-Xlinked NPs (n=3). Two-way ANOVA followed by Sidak's multiple comparisons were used for statistical analyses. Error bars represents standard error of mean (SEM);

FIG. 6A, FIG. 6B, FIG. 6C, FIG. 6D, FIG. 6E, and FIG. 6F demonstrate that XbNPs provided robust siRNA-mediated knockdown in various glioblastoma cell lines in high serum (50%) conditions. (FIG. 6A) siRNA-mediated knockdown (*:p<0.01) assessed by flow cytometry and (FIG. 6B) viability assessed by the MTS assay in GBM1A cells following treatment of crosslinked (Xlinked) and non-crosslinked (Non-Xlinked) nanoparticles using 1200 and 900 w/w formulations. (FIG. 6C) Transfection of GBM1A using XbNP formulations with varied polymer concentrations (0.8-1.1 mg/mL) and siRNA doses (60 nM and 100 nM), corresponding to 500 w/w-1200 w/w and (d) dose-dependent (10 nM-120 nM) siRNA-mediated knockdown for XbNPs. (FIG. 6E) siRNA-mediated knockdown (*:p<0.05) and (FIG. 6F) viability in GL261 cells following treatment with XbNPs using 1200, 1050, and 900 w/w formulations. Two-way ANOVA followed by Sidak's multiple comparisons were used for statistical analyses. Error bars represent standard error of mean (SEM) and n=4;

FIG. 7A, FIG. 7B, FIG. 7C, FIG. 7D, FIG. 7E, FIG. 7F, and FIG. 7G, FIG. 7H, and FIG. 7I demonstrate that XbNPs facilitated superior siRNA-mediated knockdown in murine melanoma cells (B16F10-GFP⁺) in high serum (50%) conditions compared to non-crosslinked nanoparticles (NPs) attributed by improved cellular uptake. (FIG. 7A) GFP knockdown (*:p<0.0001; n=4; two-way ANOVA followed by Sidak's multiple comparisons) assessed by flow cytometry and FIG. 7B) viability (*:p<0.0001; n=4; two-way ANOVA) assessed by MTS following treatment with crosslinked (Xlinked) and non-crosslinked (Non-Xlinked) NPs using 1200 and 900 w/w formulations with altered polymer concentration. (FIG. 7C) Representative fluorescence microscopy images of B16F10-GFPP⁺ treated with XbNPs carrying either scRNA or siRNA targeting GFP (scale bars=200 μm). (FIG. 7D) siRNA encapsulation efficiency for nanoparticles with various w/w formulations assessed by RiboGreen assay (*:p=0.020; n=2; Holm-Sidak corrected multiple Student's t-tests). (FIG. 7E) siRNA-mediated knockdown in B16F10 cells using 700 and 400 w/w NP formulations with altered siRNA dose (*:p<0.005; n=4; two-way ANOVA followed by Sidak's multiple comparisons). Assessment of (FIG. 7F) cellular uptake quantified by Cy5 spots per cell (*:p<0.0001; n=3; two-tailed Student's t-test) and (FIG. 7G) endosomal escape quantified by Gal8-GFP⁺ per cells in B16F10 cells using the Gal8-GFP recruitment assay, in which the NPs used contained Cy5-labeled siRNA. Representative images of Gl18-GFP⁺ B16F10 cells (FIG. 7H) without treatment and (FIG. 7I) after treatment with NPs (scale bars=100 μm). Error bars represent standard error of mean (SEM);

FIG. 8A, FIG. 8B, FIG. 8C, FIG. 8D, FIG. 8E, FIG. 8F, and FIG. 8G, and FIG. 8H demonstrate that XbNPs enabled organ delivery beyond the liver with preferential siRNA uptake in cancer cells leading to siRNA-mediated knockdown in tumors colonized in the lungs. (FIG. 8A) Biodistribution of nanoparticles with (Xlinked) and without (Non-Xlinked) crosslinking carrying IR-labeled siRNA using formulations of 900 w/w ratios after intravenous (i.v.) injection and (FIG. 8B) their representative IVIS images showing the fluorescent intensity of the IR-labeled siRNA. *:p<0.0005; n=5; two-way ANOVA followed by Sidak's multiple comparisons. (FIG. 8C) Biodistribution of XbNPs using 400 w/w formulation (n=3) and (FIG. 8D) a representative IVIS image of the organ accumulation after i.v. injection. (FIG. 8E) Radiant efficiency distribution based to the radiant efficiency in all of the organs for XbNPs formulations using 400 and 900 w/w. *:p<0.01; n=3-5; two-way ANOVA followed by Sidak's multiple comparisons. (FIG. 8F) Assessment of cellular uptake in different cell types in the lungs for XbNPs carrying Cy5-labeled siRNA after i.v. injection using flow cytometry. *:p=0.0033; n=5; one-way ANOVA followed by Tukey's post hoc test. (FIG. 8G) Bioluminescence intensity of B16F10-Luc⁺ cells colonized in the lungs monitored by IVIS imaging. XbNPs carrying siRNA targeting luciferase (siLuc) or Bcl-2 (siBcl-2), or scRNA as control were systemically administered 7, 9, 11, and 13 days (arrows) after tumor inoculation. *:p<0.005; n=7; two-way ANOVA followed by Dunnett's multiple comparisons. (FIG. 8H) Serum levels of aspartate aminotransferase activity (AST) and Alanine transaminase (ALT) biomarkers for liver health, following four repeated injections of XbNPs or no treatment. *:p=0.13 (AST), *:p=0.65 (ALT); n=4; two-tailed Student's t-test. Error bars represent standard error of mean (SEM);

FIG. 9A, FIG. 9B, FIG. 9C, and FIG. 9D show that photo-crosslinking generates covalent bonds in between the acrylate groups of the crosslinking polymer. (FIG. 9A) Chemical structures of the acrylate-terminated crosslinking polymer (R64Ac) and the amine-terminated polymer (R646). ¹H NMR Spectra of nanoparticles (NPs) being (FIG. 9B) crosslinked (Xlinked) and (FIG. 9C) non-crosslinked (Non-Xlinked) without and (FIG. 9D) with UV-exposure. Integration ratios of Ac (dotted box) to a, b, and c peaks showed a 79.1% (±0.3) decrease of acrylate bonds after photo-crosslinking;

FIG. 10A, FIG. 10B, and FIG. 10C show that tuning the physical properties of XbNPs by altering siRNA payload and UV exposure time. Nanoparticle (FIG. 10A) size and (FIG. 10B) surface charged assessed by dynamic light scattering (DLS) for formulations with altered siRNA dose. (FIG. 10C) Surface charge of nanoparticles after varied UV exposure time. One-way ANOVA followed by Tukey's post hoc test were used for statistical analyses, *:p<0.05 considered statistically different. Error bar represents standard error of mean (SEM) and n=3;

FIG. 11A, FIG. 11B, FIG. 11C, FIG. 11D, and FIG. 11E demonstrate that in vitro transfection of patient-derived glioblastoma cells (GBM319-GFP⁺) in 50% serum media. (FIG. 11A) siRNA-mediated knockdown for crosslinked (Xlinked) and Non-Xlinked nanoparticles (NPs) formulations using 1200 and 600 w/w. Viability following treatment of (FIG. 11B) Xlinked and Non-Xlinked formulations with varied ratios of acrylate-terminated (Ac) and amine-endcapped (E) polymers, (FIG. 11C) XbNP formulations with altered polymer concentration, (FIG. 11D) XbNP formulations with altered siRNA dose, and (FIG. 11E) Irgacure 2959 (Irg) by itself at varied concentrations. Error bars represent standard error of mean (SEM) and n=4;

FIG. 12A and FIG. 12B demonstrate that XbNPs enhanced cellular uptake in patient-derived glioblastoma cells (GBM319) following transfection in high serum conditions (50% and 100% serum media). Mean fluorescent intensity of nanoparticle (Cy5+) uptake in GBM319 cells assessed by flow cytometry at (FIG. 12A) 6 h (p<0.0001; n=4) and (FIG. 12B) 24 h (p<0.0001; n=4) after treatment with crosslinked (Xlinked) and non-crosslinked (Non-Xlinked) nanoparticle formulations using 1200 and 900 w/w ratios. Two-way ANOVA followed by Sidak's multiple comparisons used for statistical analyses. Error bars represent standard error of mean (SEM);

FIG. 13A and FIG. 13B demonstrate that XbNPs enabled siRNA delivery to organs beyond the liver following systemic administration. (FIG. 13A) Representative LI-COR images of the IR-intensity in the harvested organs after intravenous (i.v.) injection of nanoparticles with (Xlinked) and without (Non-Xlinked) crosslinking carrying IR-labeled siRNA using 900 w/w formulations and (FIG. 13B) the measured biodistribution with n=5 (*:p<0.0001; two-way ANOVA followed by Sidak's multiple comparisons). Error bars represent standard error of mean (SEM); and

FIG. 14A, FIG. 14B, and FIG. 14C shows a lung metastatic mouse model using B16F10-Luc⁺ cells. (FIG. 14A) Bioluminescence signal in the lungs assessed by IVIS following tumor inoculation via intravenous injection of B16F10-Luc⁺ cells (n=5). (FIG. 14B) IVIS image of the bioluminescence signal 14 days after tumor inoculation. (FIG. 14C) IVIS image showing the bioluminescence of the collected organs 14 days after tumor inoculation. Error bars represent standard error of mean (SEM).

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figure. Therefore, it is to be understood that the presently disclosed subject matter is not to be limited to the specific embodiments disclosed and that modifications and other embodiments are intended to be included within the scope of the appended claims.

### A. Photo-Crosslinked Bioreducible Polymeric Nanoparticles

In some embodiments, the presently disclosed subject matter provides a nanoparticle comprising a bioreducible cationic polymer, a crosslinking polymer and one or more nucleic acids, wherein the bioreducible cationic polymer and crosslinking polymer are crosslinked and the one or more nucleic acids are encapsulated within the nanoparticle.

In some embodiments, the bioreducible cationic polymer comprises a bioreducible poly(beta-amino ester). In certain embodiments, the bioreducible poly(beta-amino ester) comprises at least one degradable ester bond and at least one disulfide bond. In particular embodiments, the bioreducible poly(beta-amino ester) comprises an amine-terminated bioreducible poly(beta-amino ester). Representative bioreducible cationic polymers are disclosed in U.S. Patent Application Publication No. 20150273071 for Bioreducible Poly(Beta-Amino Ester) s for siRNA Delivery, to Green et al., published Oct. 1, 2015, which is incorporated herein by reference in its entirety.

Generally, the presently disclosed bioreducible cationic polymers include a backbone derived from a diacrylate monomer (designated herein below as “B”), an amino-alcohol side chain monomer (designated herein below as “S”), and an amine-containing end-capping monomer (designated herein below as “E”). The end group structures are distinct and separate from the polymer backbone structures and the side chain structures of the intermediate precursor molecule for a given polymeric material. The presently disclosed PBAE compositions can be designated, for example, as BR5-S4-E7 or R547, where BR is the bioreducible backbone and S is the side chain, followed by the number of carbons in their hydrocarbon chain. Endcapping monomers, E, are sequentially numbered according to similarities in their amine structures.

In some embodiments, the amine-terminated bioreducible poly(beta-amino ester) comprises a monomer having the following structure:

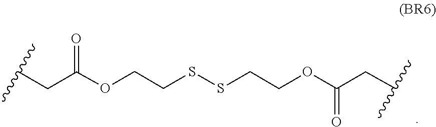 [SMILES: *CC(=O)OCCSSCCOC(=O)C*]

In some embodiments, the amine-terminated bioreducible poly(beta-amino ester) comprises a compound having the following structure:

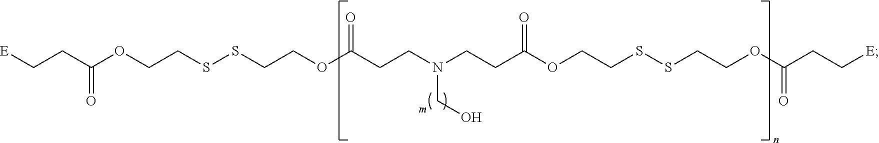

wherein:
n is an integer from 1 to 10,000;
m is an integer selected from the group consisting of 2, 3, 4, 5, 6, 7, 8, and 9; and
E is an amine-containing endcapping group.

Side chain monomers, e.g., N—(CH₂)ₘ—OH, may further comprise a C₂ to C₉ linear or branched alkylene, including C₂-C₉ straightchain or branched alkylene, including C₂, C₃, C₄, C₅, C₆, C₇, C₈, and C₉ alkylene, which is optionally substituted. Illustrative substituents include hydroxyl, alkyl, alkenyl, thiol, amine, carbonyl, halogen, and fluorinated alkylene.

In representative embodiments, the side chain monomer is selected from the group consisting of:

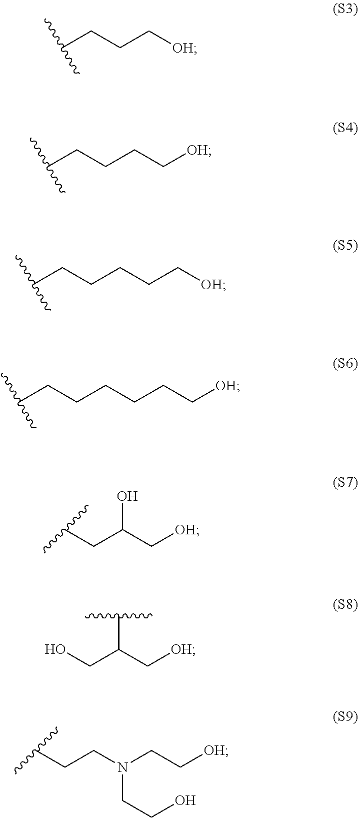

In certain embodiments, the amine-containing endcapping group is selected from the group consisting of:

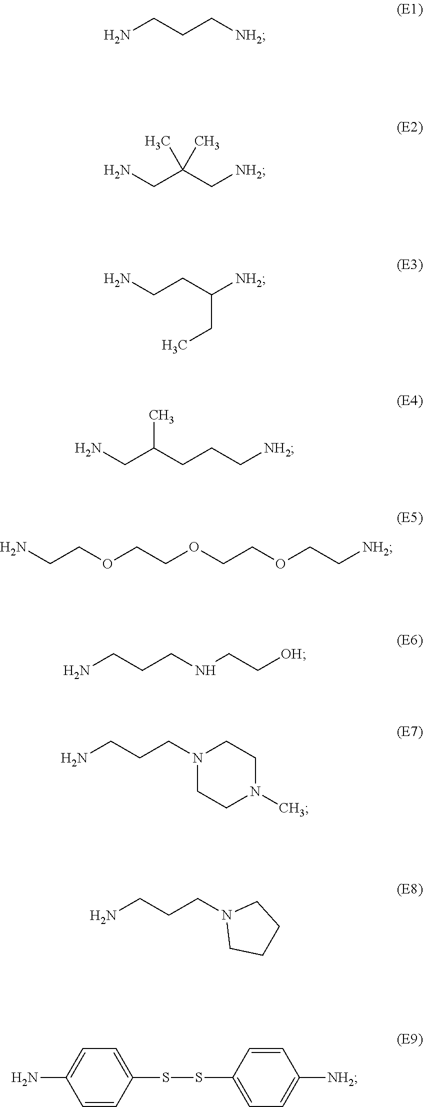

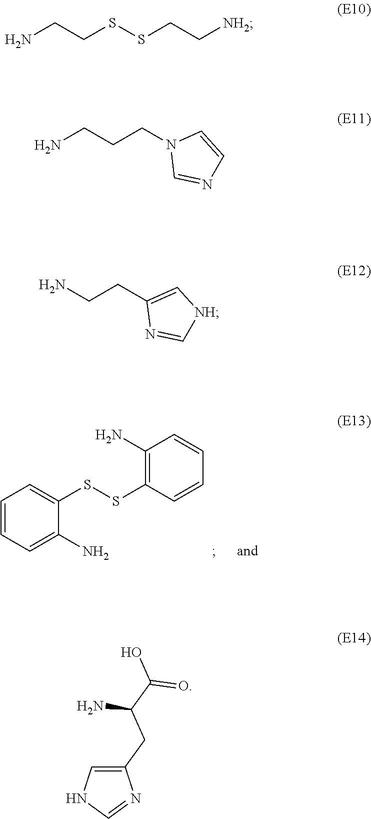 [SMILES: NCCSSCCN ; NCCCn1ccnc1 ; NCCc1c[nH]cn1 ; Nc1ccccc1SSc1ccccc1N ; N[C@H](Cc1c[nH]cn1)C(=O)O]

Other representative endcapping groups include:

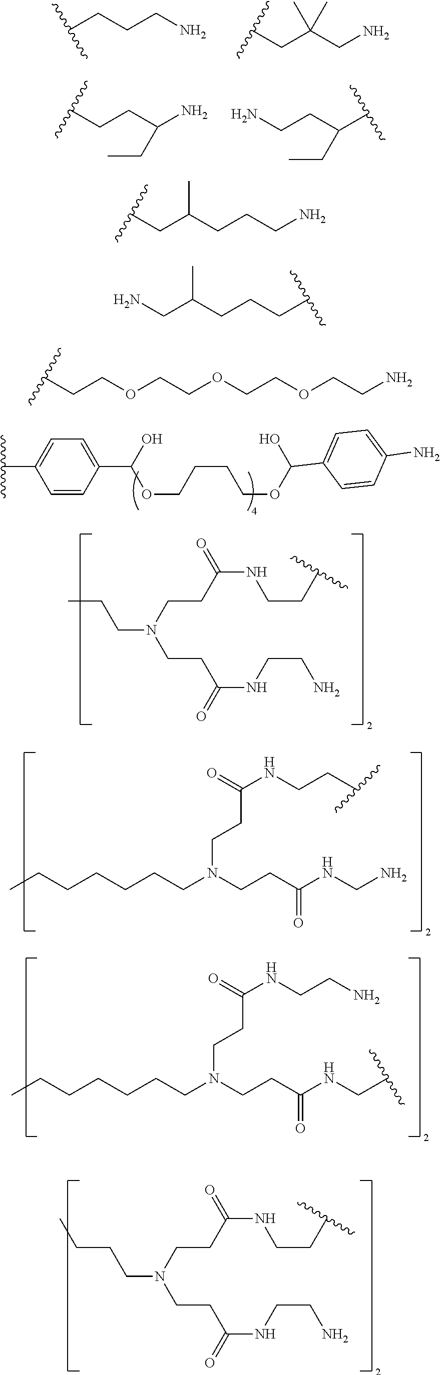

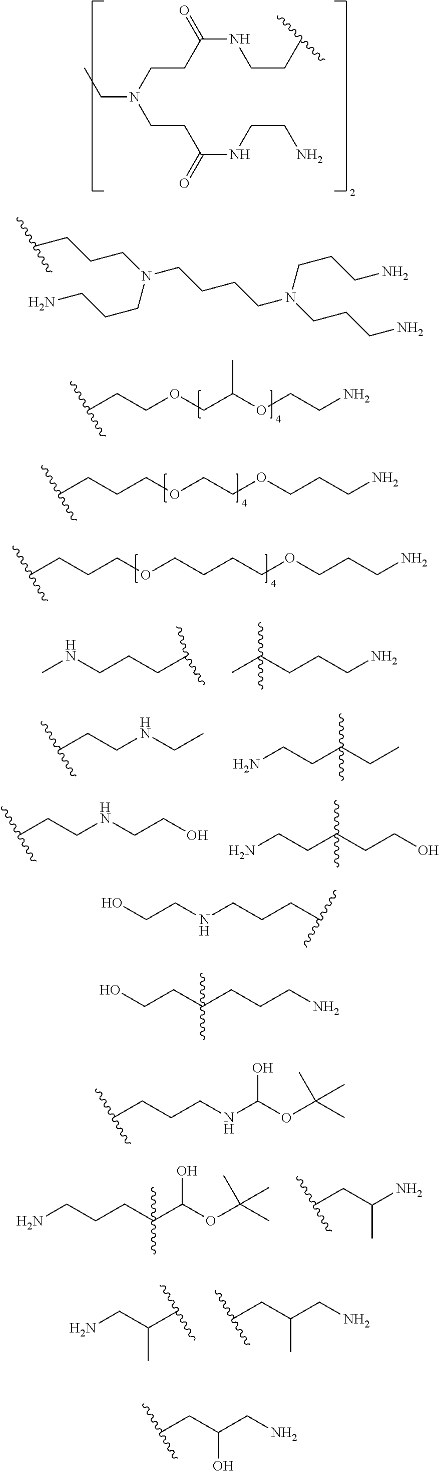

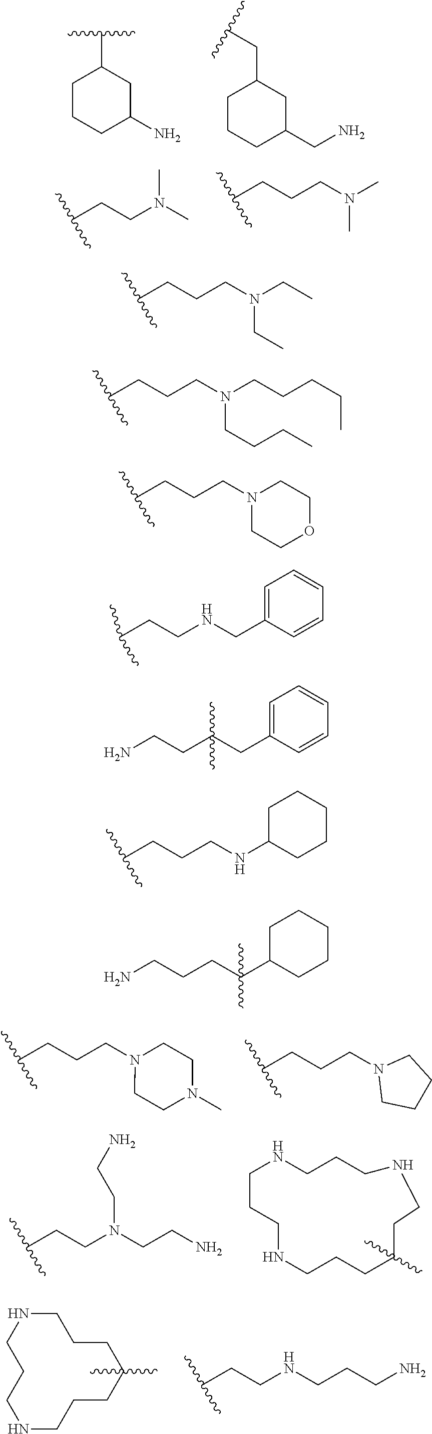

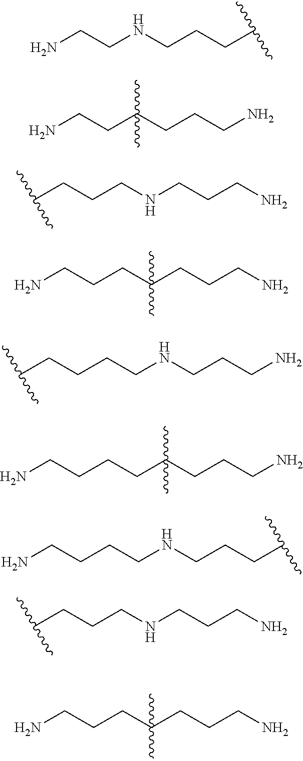

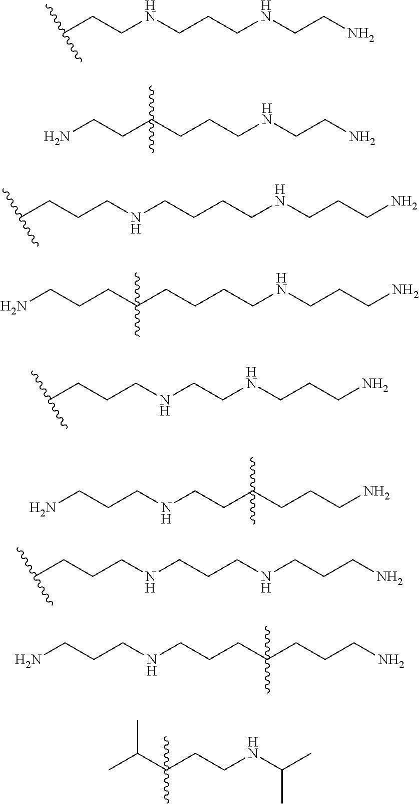

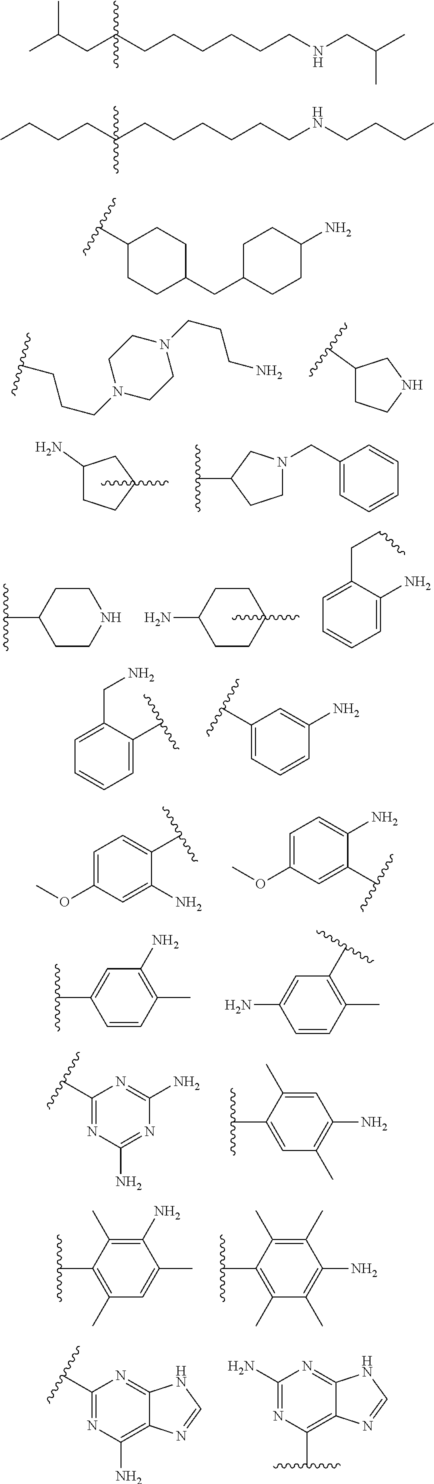

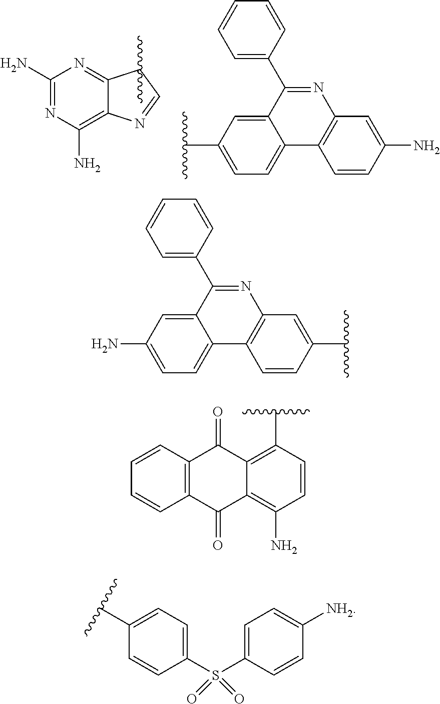

Yet other endcapping groups include:

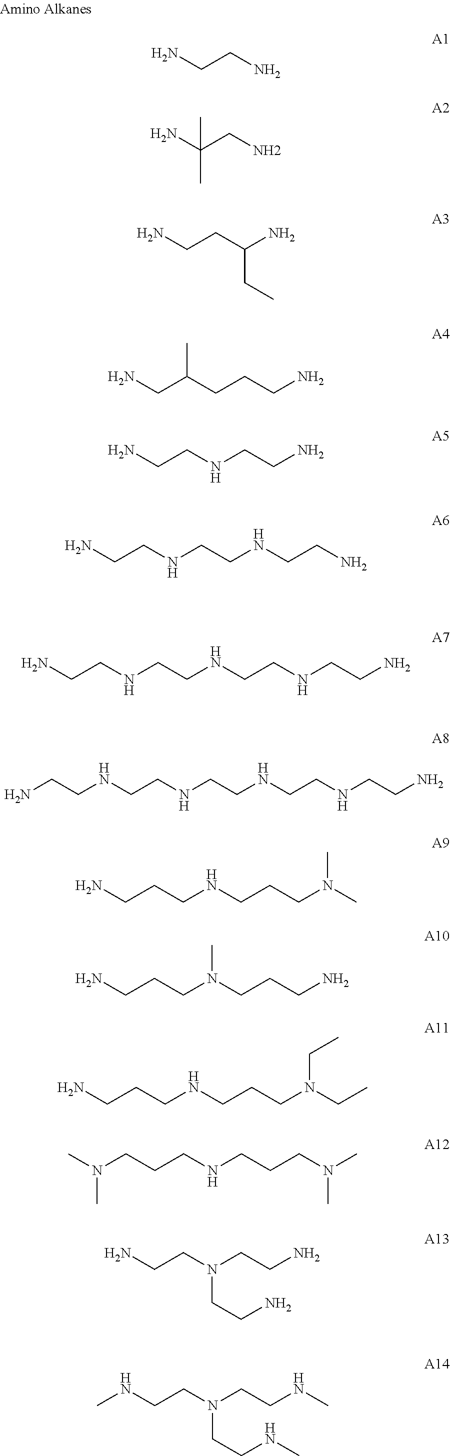

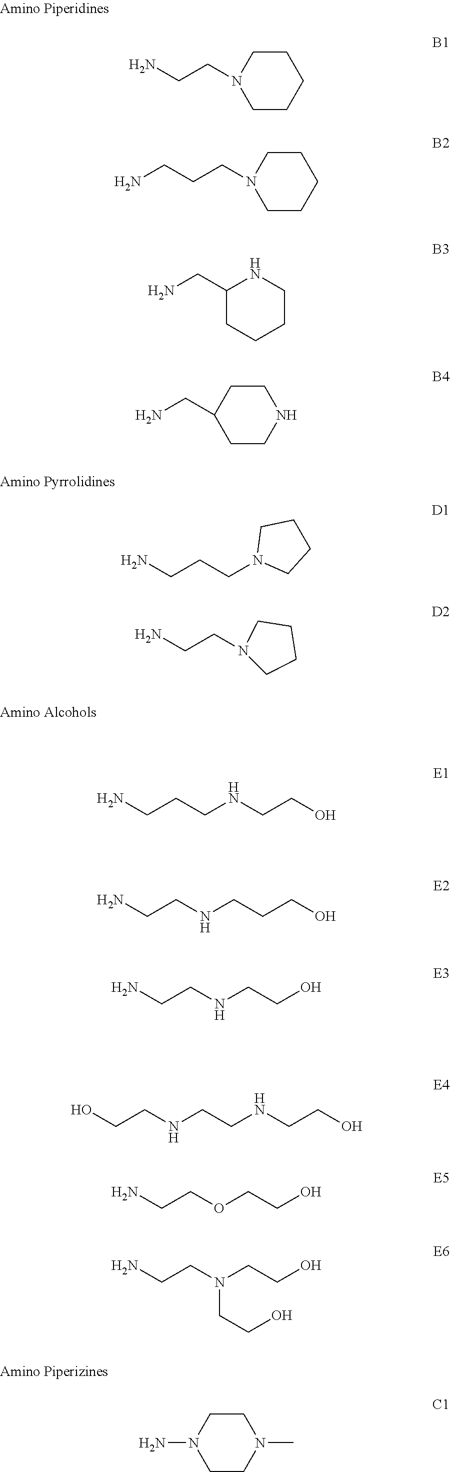

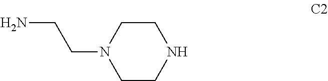

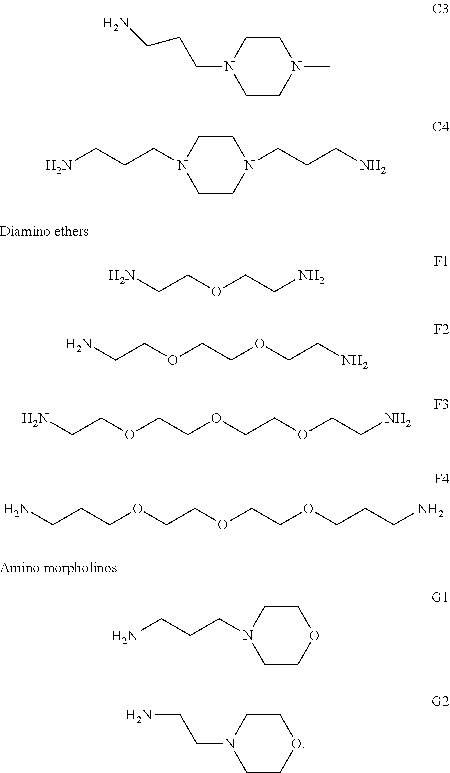

Additional endcapping groups include:

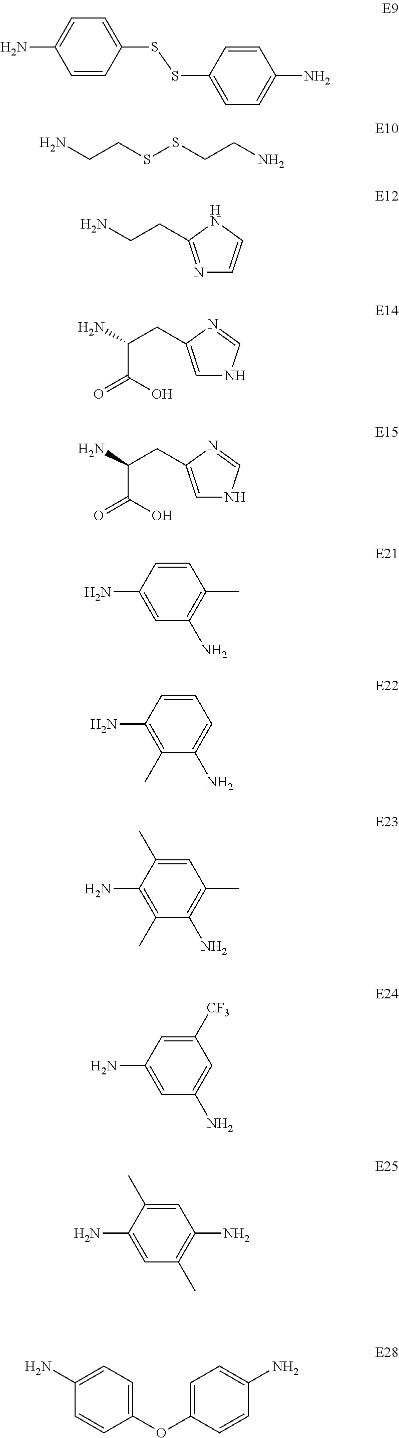

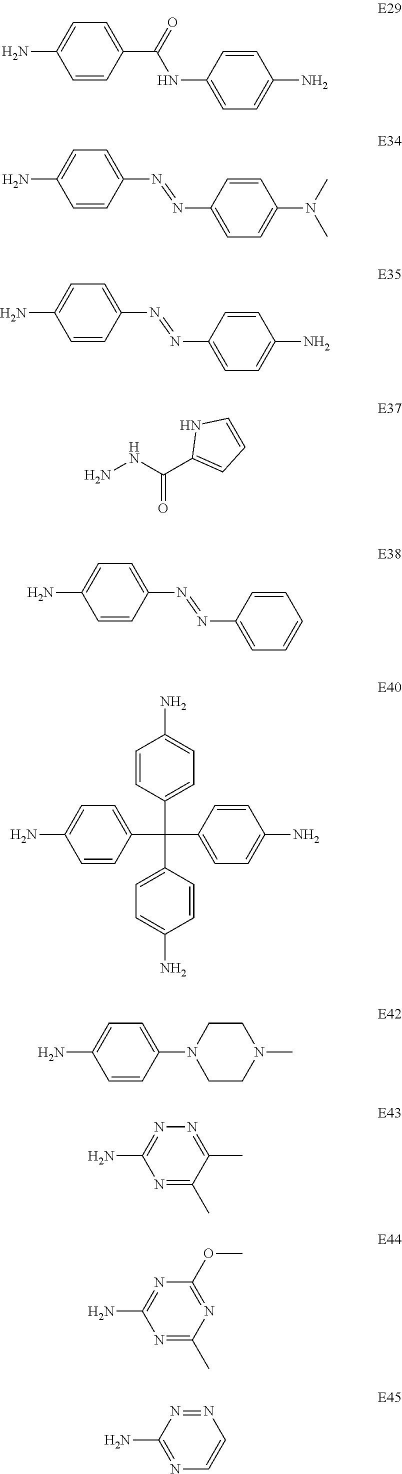

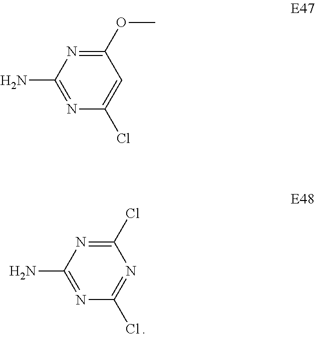

In particular embodiments, the amine-terminated bioreducible poly(beta-amino ester) is selected from the group consisting of R646 and R647:

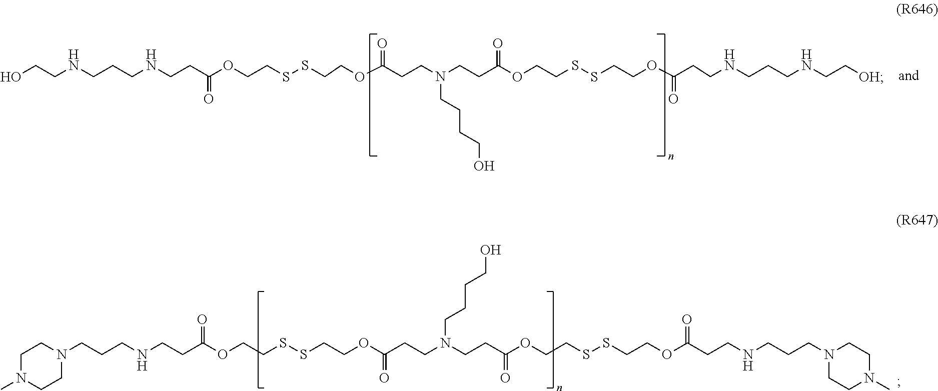

wherein n is an integer from 1 to 10,000.

In particular embodiments, n is selected from the group consisting of: an integer from 1 to 1,000; an integer from 1 to 100; an integer from 1 to 30; an integer from 5 to 20; an integer from 10 to 15; and an integer from 1 to 10.

In some embodiments, the crosslinking polymer comprises an acrylate-terminated bioreducible poly(beta-amino ester) having at least one disulfide bond. In certain embodiments, the acrylate-terminated bioreducible poly(beta-amino ester) comprises a monomer having the following structure:

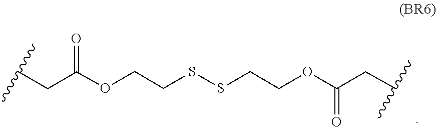 [SMILES: *CC(=O)OCCSSCCOC(=O)C*]

In particular embodiments, the acrylate-terminated bioreducible poly(beta-amino ester) comprises a compound having the following structure:

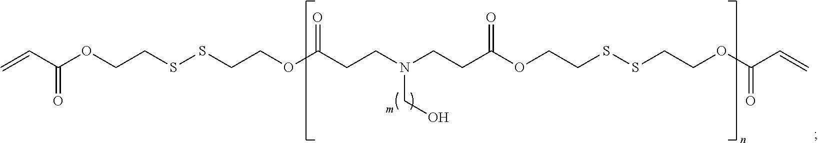

wherein:
n is an integer from 1 to 10,000; and
m is an integer selected from the group consisting of 2, 3, 4, 5, 6, 7, 8, and 9.

In yet more particular embodiments, the acrylate-terminated bioreducible poly(beta-amino ester) comprises R64-Ac:

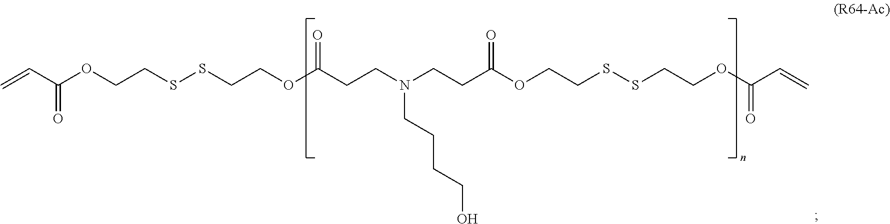

wherein n is an integer from 1 to 10,000.

In some embodiments, the crosslinking polymer and the cationic polymer have a mass ratio (weight ratio) of about 1:1 to 1:5 (w/w). In certain embodiments, the crosslinking polymer and the cationic polymer have a mass ratio (weight ratio) of about 1:3 (w/w).

In some embodiments, the one or more nucleic acids are selected from the group consisting of DNA, RNA, siRNA, mRNA, miRNA, isRNA, agRNA, and smRNA. In certain aspects, the one or more nucleic acids comprises siRNA.

In some embodiments, the nanoparticle has a polymer:nucleic acid mass ratio (weight/weight or w/w) ranging from about 20 (w/w) polymer:nucleic acid to about 2,000 (w/w) polymer:nucleic acid.

In some embodiments, the nanoparticle has a substantially neutral surface charge.

In some embodiments, the nanoparticle has a particle size ranging from about 50 nm to 500 nm, e.g., about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 nm in size.

In embodiments, the particle has at least one dimension in the range of about 50 nm to about 500 nm, or from about 50 to about 200 nm. Exemplary particles may have an average size (e.g., average diameter) of about 50, about 75, about 100, about 125, about 150, about 200, about 250, about 300, about 400 or about 500 nm. In some embodiments, the nanoparticle has an average diameter of from about 50 nm to about 500 nm, from about 50 nm to about 300 nm, or from about 50 nm to about 200 nm, or from about 50 nm to about 150 nm, or from about 70 to 100 nm. In embodiments, the nanoparticle has an average diameter of from about 200 nm to about 500 nm. In embodiments, the nanoparticle has at least one dimension, e.g., average diameter, of about 50 to about 100 nm. In certain embodiments, the nanoparticle has a particle size ranging from about 100 nm to 250 nm.

In other embodiments, the presently disclosed subject matter provides a pharmaceutical formulation comprising the presently disclosed nanoparticle and a pharmaceutically acceptable carrier.

As used herein, “pharmaceutically acceptable carrier” is intended to include, but is not limited to, water, saline, dextrose solutions, human serum albumin, liposomes, hydrogels, microparticles and nanoparticles. The use of such media and agents for pharmaceutically active compositions is well known in the art, and thus further examples and methods of incorporating each into compositions at effective levels need not be discussed here.

In particular embodiments, the pharmaceutical formulation further comprises one or more therapeutic agents.

### B. Methods for Preparing Photo-Crosslinked Bioreducible Polymeric Nanoparticles

In yet other embodiments, the presently disclosed subject matter provides a method for preparing a crosslinked nanoparticle, the method comprising:
(a) mixing a bioreducible cationic polymer, and crosslinking polymer, and one or more nucleic acids to form a self-assembled bioreducible nanoparticle;
(b) incubating the self-assembled bioreducible nanoparticle for a period of time;
(c) adding a photoinitiator to the self-assembled bioreducible nanoparticle; and
(d) exposing the self-assembled bioreducible nanoparticle and photoinitiator to ultraviolet light to form a crosslinked bioreducible nanoparticle comprising the one or more nucleic acids.

In some embodiments, the cationic polymer comprises a bioreducible poly(beta-amino ester). In certain embodiments, the bioreducible poly(beta-amino ester) comprises at least one degradable ester bond and at least one disulfide bond. In particular embodiments, the bioreducible poly(beta-amino ester) comprises an amine-terminated bioreducible poly(beta-amino ester).

In particular embodiments, the method further comprises synthesizing the amine-terminated bioreducible poly(beta-amino ester) with a BR6 monomer:

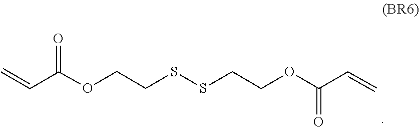 [SMILES: C=CC(=O)OCCSSCCOC(=O)C=C]

In more particular embodiments, the method further comprises synthesizing the amine-terminated bioreducible poly(beta-amino ester) with a BR6 monomer and a linear amino alcohol monomer of the general formula NH₂—R—OH, where R comprises an alkyl chain consisting of 2, 3, 4, 5, 6, 7, 8, or 9 carbons.

In yet more particular embodiments, the amine-terminated bioreducible poly(beta-amino ester) comprises a compound having the following structure:

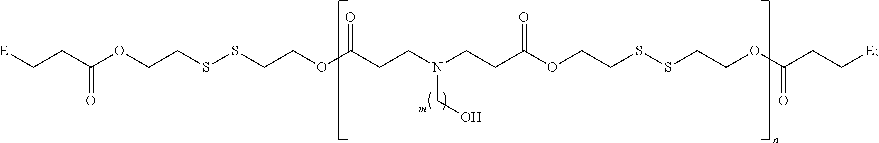

wherein:
n is an integer from 1 to 10,000;
m is an integer selected from the group consisting of 2, 3, 4, 5, 6, 7, 8, and 9; and
E is an amine-containing endcapping group.

In certain embodiments, the amine-containing endcapping group is selected from the group consisting of:

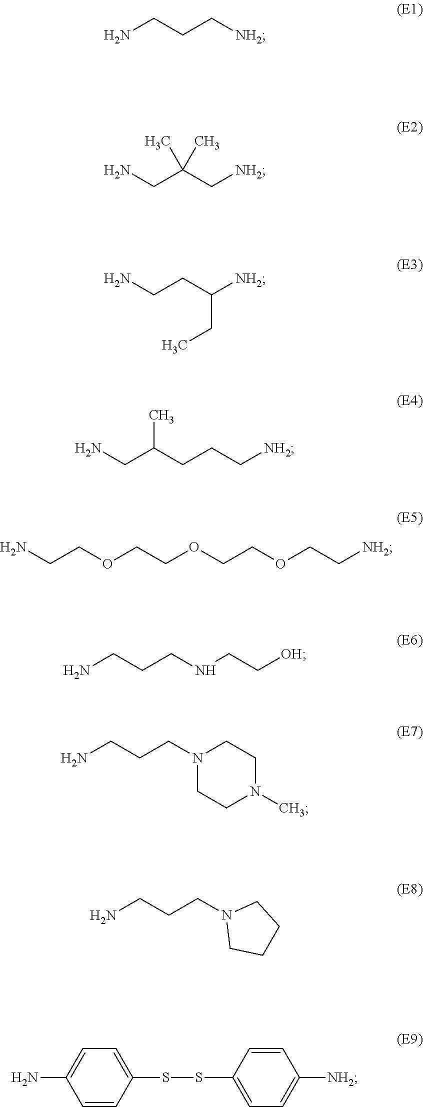

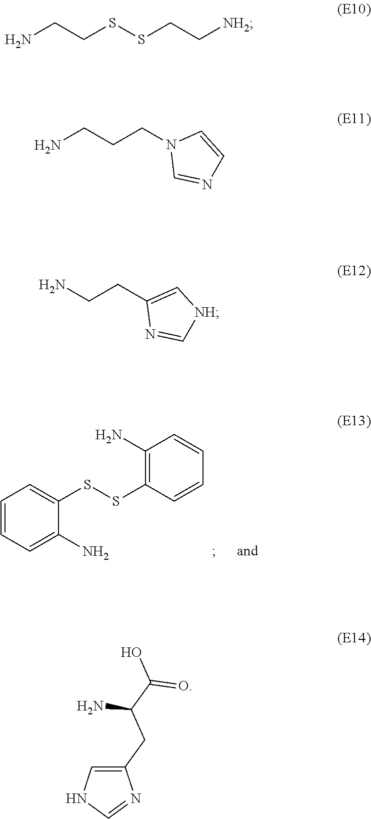 [SMILES: NCCSSCCN ; NCCCn1ccnc1 ; NCCc1c[nH]cn1 ; Nc1ccccc1SSc1ccccc1N ; N[C@H](Cc1c[nH]cn1)C(=O)O]

Other representative endcapping groups include:

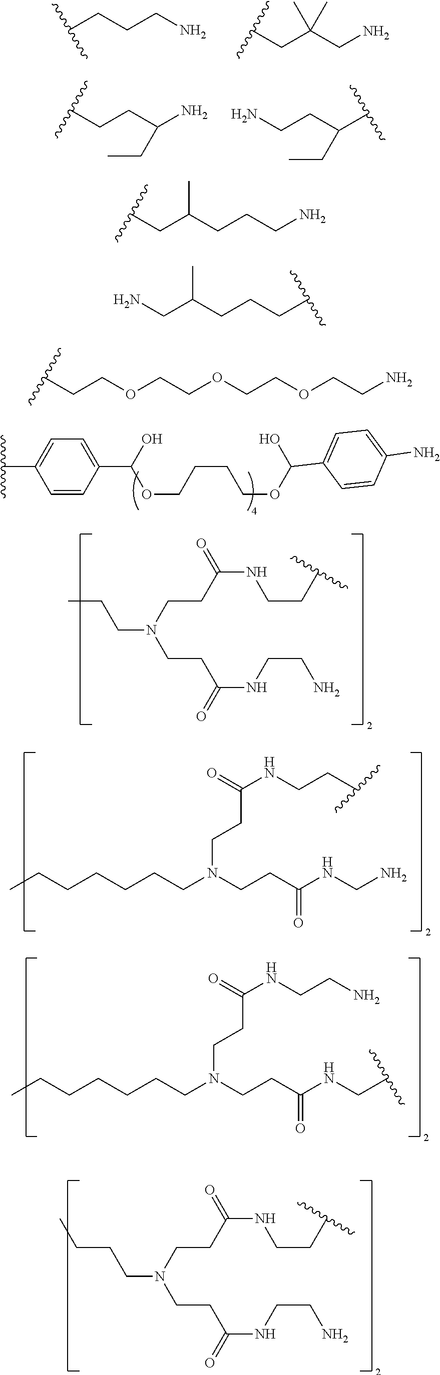

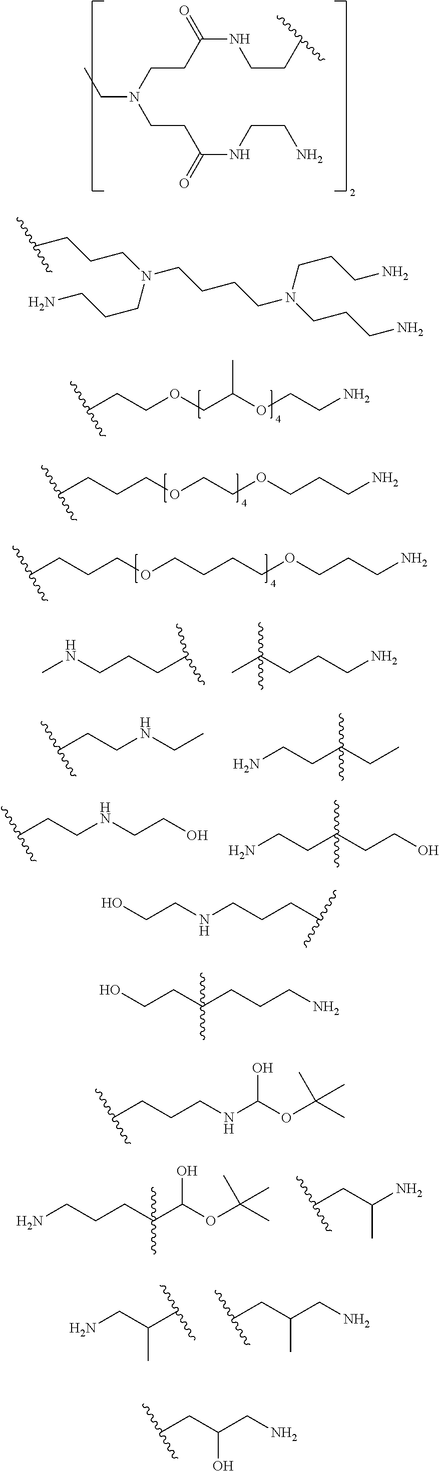

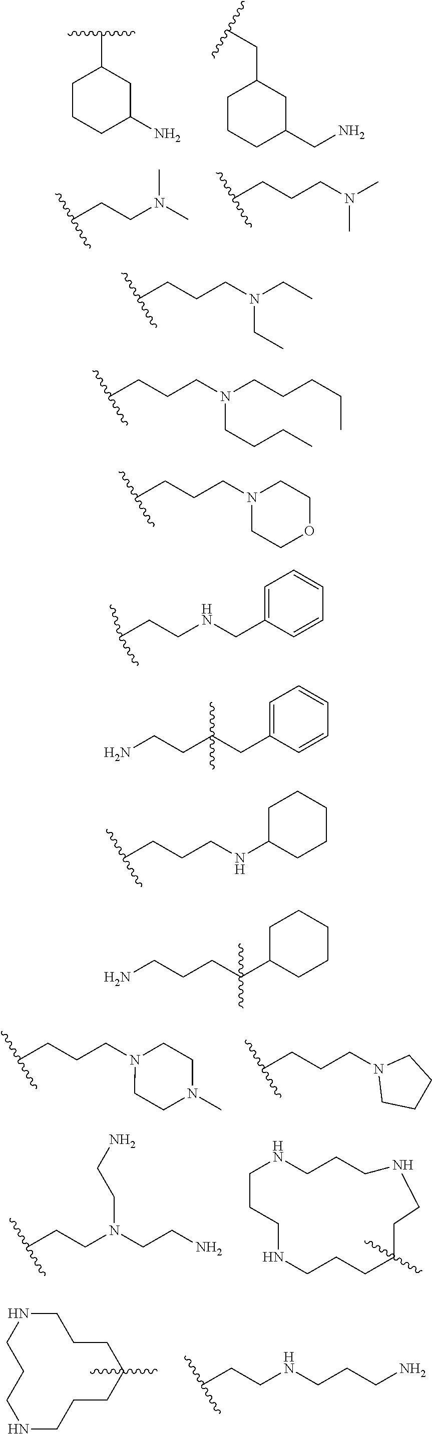

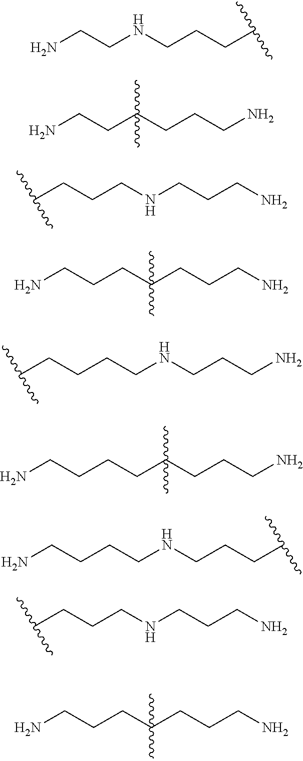

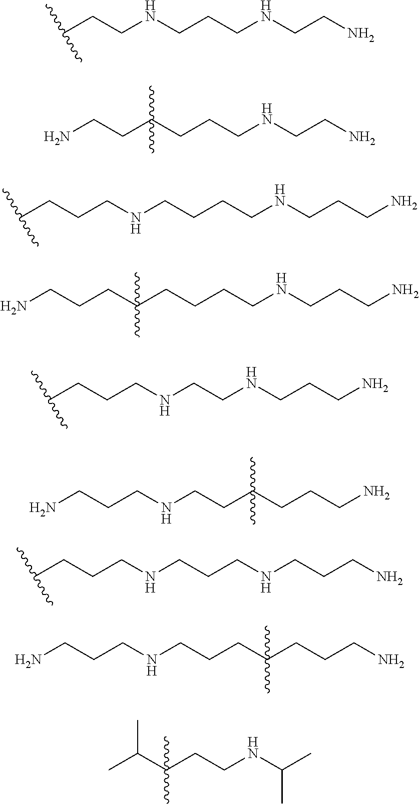

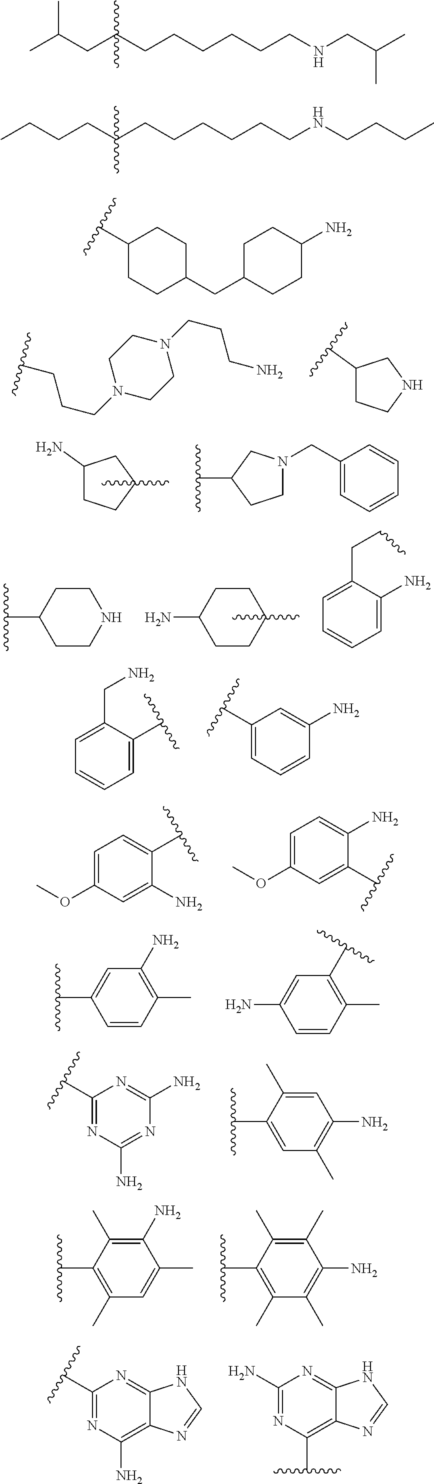

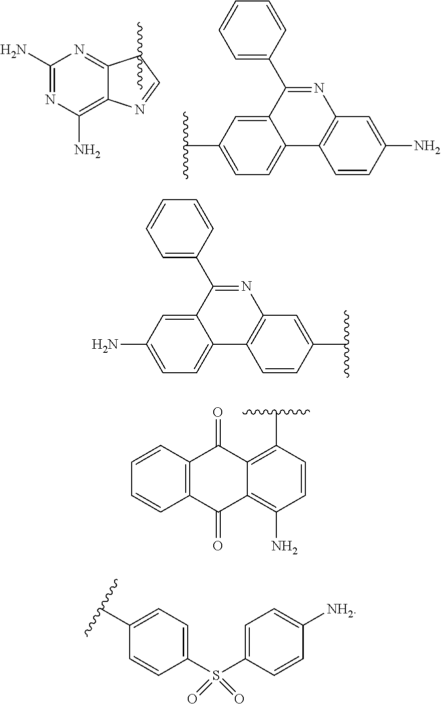

Yet other endcapping groups include:

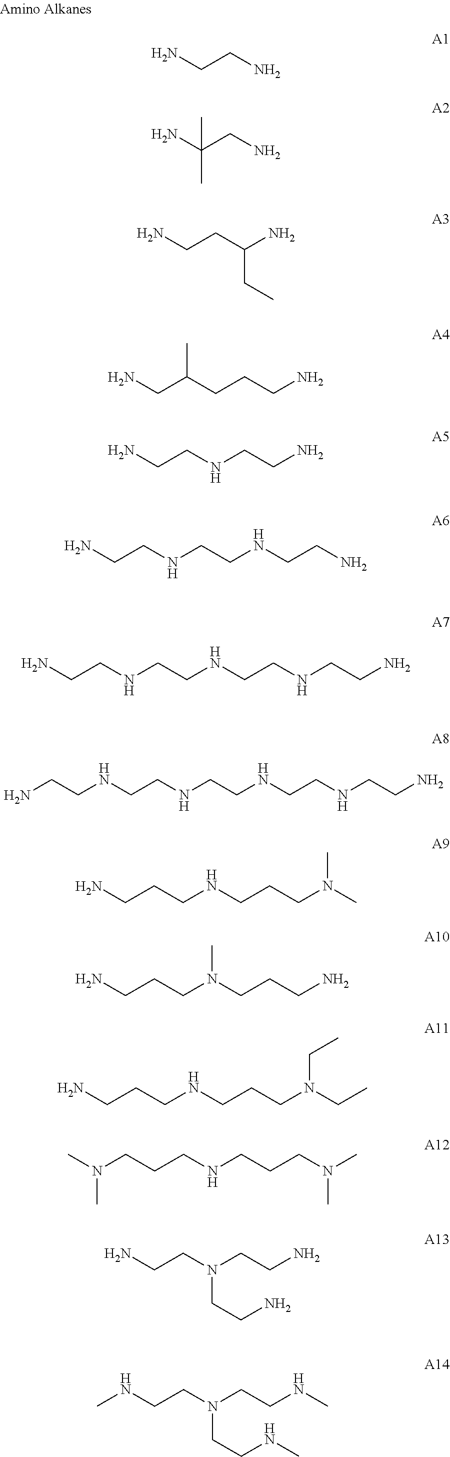

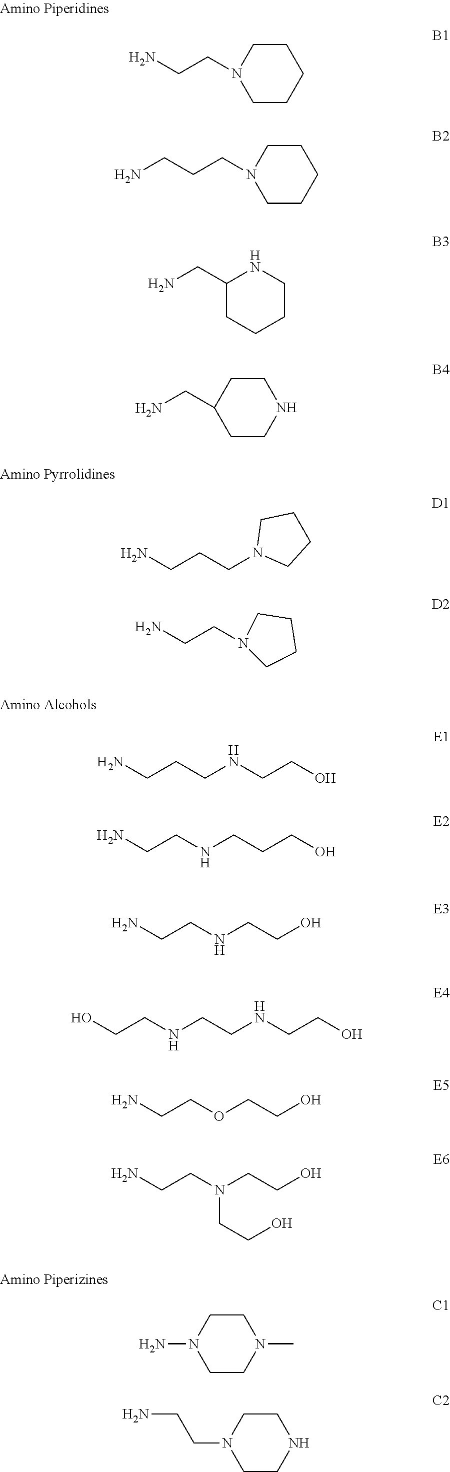

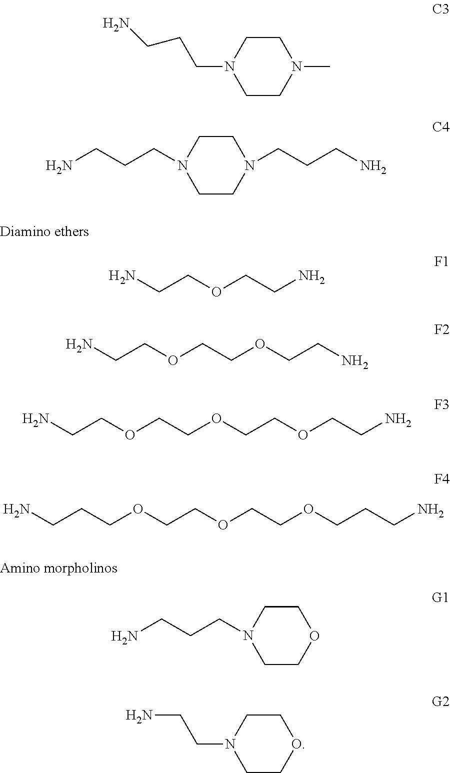

Additional endcapping groups include:

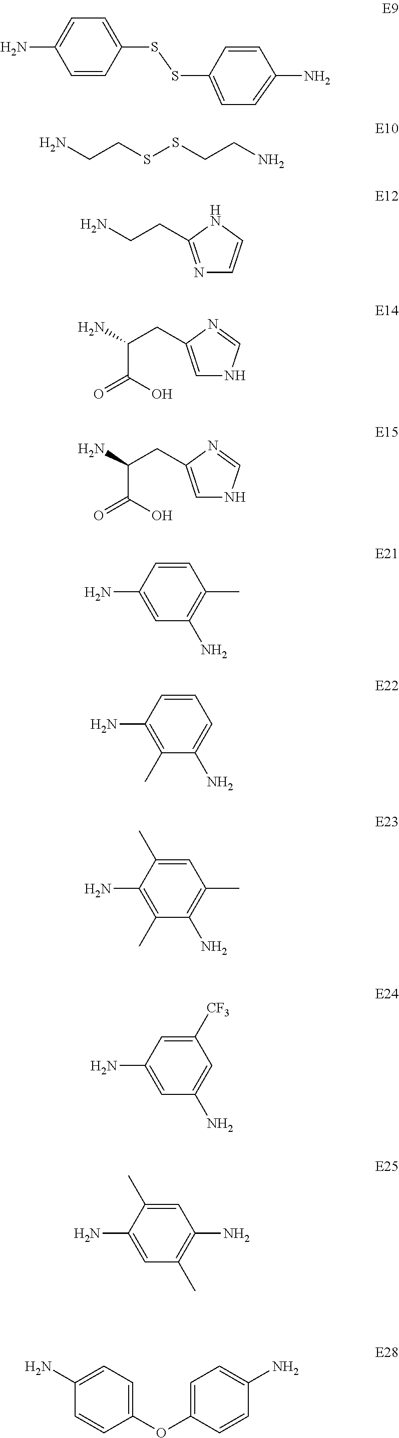

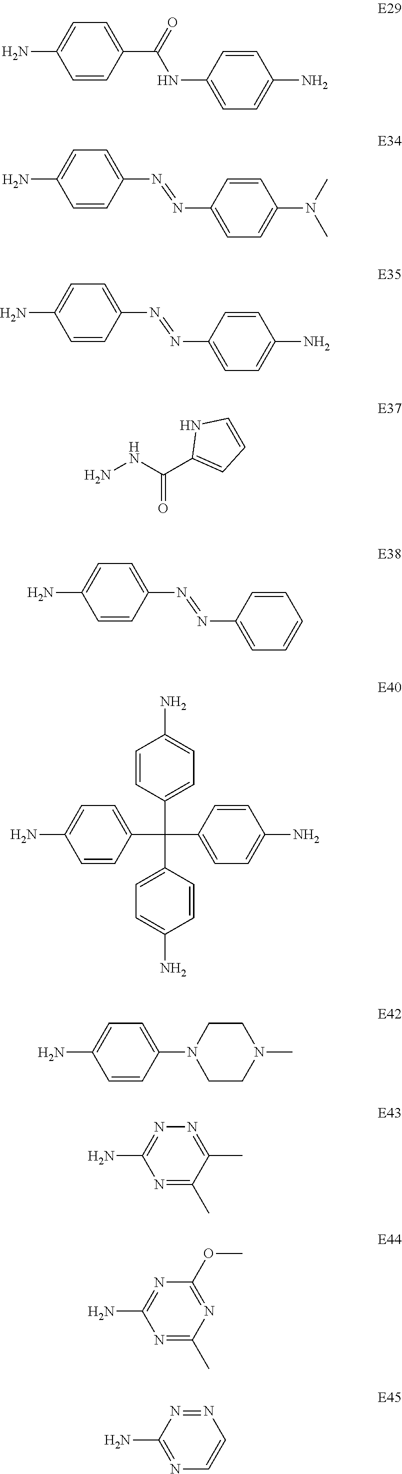

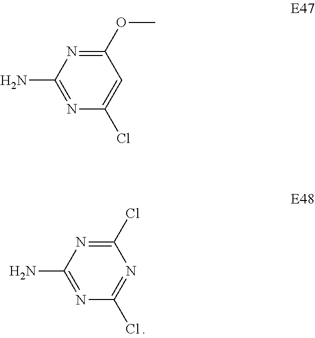

In particular embodiments, the amine-terminated bioreducible poly(b-amino ester) is selected from the group consisting of R646 and R647:

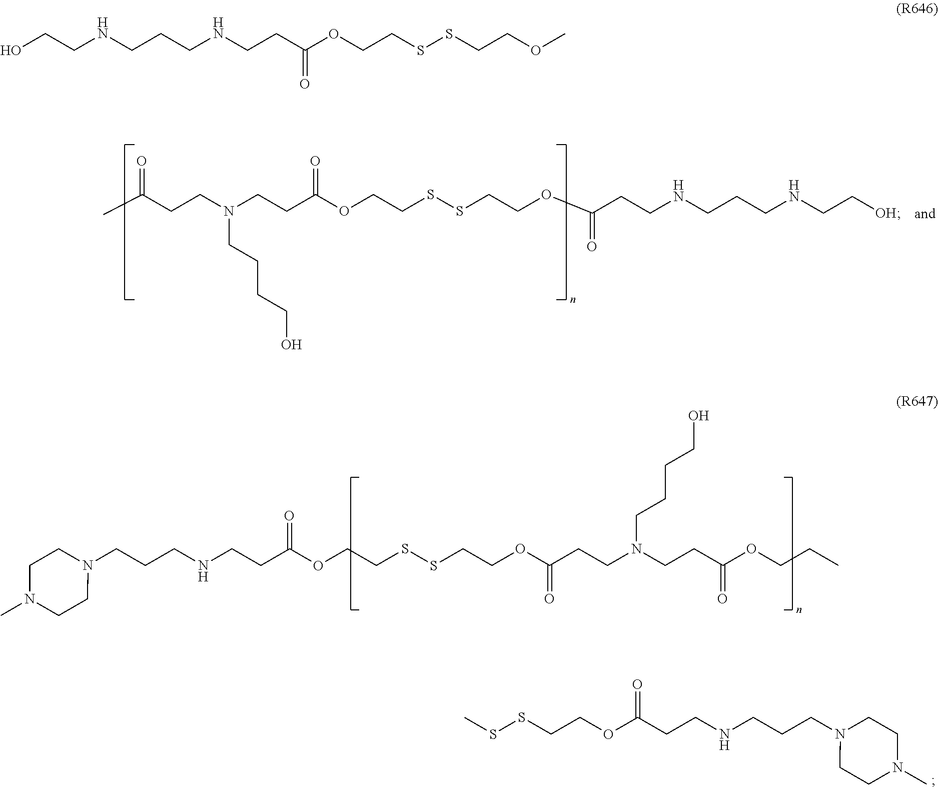

wherein n is an integer from 1 to 10,000.

In some embodiments, the crosslinking polymer comprises an acrylate-terminated bioreducible poly(beta-amino ester) having at least one disulfide bond.

In some embodiments, the method further comprises synthesizing the acrylate-terminated bioreducible poly(beta-amino ester) with a BR6 monomer:

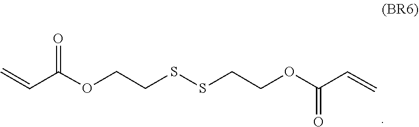 [SMILES: C=CC(=O)OCCSSCCOC(=O)C=C]

In some embodiments, the acrylate-terminated bioreducible poly(beta-amino ester) is synthesized using the BR6 monomer and a linear amino alcohol monomer having the general formula of NH₂—R—OH, where R is an alkyl chain consisting of 2, 3, 4, 5, 6, 7, 8, or 9 carbons.

In certain embodiments, the acrylate-terminated bioreducible poly(b-amino ester) comprises R64-Ac:

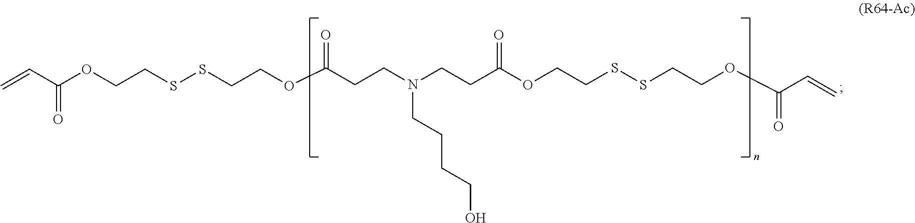

wherein n is an integer from 1 to 10,000.

In some embodiments, the crosslinking polymer and the cationic polymer have a mass ratio (weight ratio) of about 1:1 to 1:5 (w/w). In certain embodiment, the crosslinking polymer and the cationic polymer have a mass ratio (weight ratio) of about 1:3 (w/w).

In some embodiments, the one or more nucleic acids are selected from the group consisting of DNA, RNA, siRNA, mRNA, miRNA, isRNA, agRNA, and smRNA. In certain embodiments, the nucleic acid comprises siRNA.

In some embodiments, the nanoparticle has a polymer:nucleic acid mass ratio (weight/weight or w/w) ranging from about 20 (w/w) polymer:nucleic acid to about 2,000 (w/w) polymer:nucleic acid.

C. Methods for Treating a Cancer with Photo-Crosslinked Bioreducible Polymeric Nanoparticles

In yet other embodiments, the presently disclosed subject matter provides a method for treating a cancer, the method comprising administering to a subject in need of treatment thereof a therapeutically effective amount of the presently disclosed nanoparticle or a pharmaceutical formulation thereof to treat the cancer.

In some embodiments, the nanoparticle enters a cytosol of a cell of the subject. In some embodiments, the at least one disulfide bond of the bioreducible poly(beta-amino ester) is reductively degraded in the cytosol to release the nucleic acid in the cytosol of the cell of the subject. In some embodiments, the at least one disulfide bond is reductively degraded via glutathione.

In some embodiments, the method comprises systemically administering the nanoparticle or pharmaceutical formulation. In some embodiments, the one or more nanoparticles are delivered to one or more organs beyond the liver. In certain embodiments, the one or more organs beyond the liver comprises the lungs.

In some embodiments, the method comprises preferential uptake of the nucleic acid in one or more cancer cells. In some embodiments, the crosslinked nanoparticle exhibits enhanced colloidal stability in the bloodstream compared to a non-crosslinked nanoparticle. In some embodiments, the crosslinked nanoparticle exhibits reduced adsorption of anionic serum proteins compared to a non-crosslinked nanoparticle.

In some embodiments, the cancer is selected from the group consisting of brain cancer, melanoma, lung cancer, breast cancer, prostate cancer, and colorectal cancer. In certain embodiments, the brain cancer comprises a glioblastoma.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

As used herein, the term “inhibit,” and grammatical derivations thereof, refers to the ability of a presently disclosed compound, e.g., a presently disclosed compound of formula (I), to block, partially block, interfere, decrease, or reduce the growth and/or metastasis of a cancer cell. Thus, one of ordinary skill in the art would appreciate that the term “inhibit” encompasses a complete and/or partial decrease in the growth and/or metastasis of a cancer cell, e.g., a decrease by at least 10%, in some embodiments, a decrease by at least 20%, 30%, 50%, 75%, 95%, 98%, and up to and including 100%.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly a compound of formula (I) and at least one therapeutic agent and/or imaging agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compositions described herein can be administered alone or in combination with adjuvants that enhance stability of the compositions alone or in combination with one or more therapeutic agents and/or imaging agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a presently disclosed composition and at least one additional therapeutic agent can be varied so long as the beneficial effects of the combination of these agents are achieved. Accordingly, the phrase “in combination with” refers to the administration of a presently disclosed composition and at least one additional therapeutic agent either simultaneously, sequentially, or a combination thereof. Therefore, a subject administered a combination of a presently disclosed composition and at least one additional therapeutic agent can receive a presently disclosed composition of and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the presently disclosed composition and at least one additional therapeutic agent are administered simultaneously, they can be administered to the subject as separate pharmaceutical compositions, each comprising either a presently disclosed composition or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a presently disclosed composition and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + {Q}_{b} / {Q}_{B} = Synergy Index ( SI )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, +100% in some embodiments+50%, in some embodiments+20%, in some embodiments+10%, in some embodiments+5%, in some embodiments+1%, in some embodiments+0.5%, and in some embodiments+0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

Photo-Crosslinked Bioreducible Polymeric Nanoparticles for Enhanced Systemic siRNA Delivery as Cancer Therapy

### 1.1 Overview

Clinical translation of polymer-based nanocarriers for systemic delivery of RNA has been limited due to poor colloidal stability in the blood stream and intracellular delivery of the RNA to the cytosol. To address these limitations, the presently disclosed subject matter provides a new strategy incorporating photo-crosslinking of bioreducible nanoparticles for improved stability extracellularly and rapid release of RNA intracellularly. In the presently disclosed design, the polymeric nanocarriers contain ester bonds for hydrolytic degradation and disulfide bonds for environmentally triggered siRNA release in the cytosol. These photo-crosslinked bioreducible nanoparticles (XbNPs) have a shielded surface charge, reduced adsorption of serum proteins, and enable superior siRNA-mediated knockdown in both glioma and melanoma cells in high-serum conditions compared to non-crosslinked formulations.

Mechanistically, XbNPs promote cellular uptake and the presence of secondary and tertiary amines enables efficient endosomal escape. Following systemic administration, XbNPs facilitate targeting of cancer cells and tissue-mediated siRNA delivery beyond the liver, unlike conventional nanoparticle-based delivery. These attributes of XbNPs facilitate robust siRNA-mediated knockdown in vivo in melanoma tumors colonized in the lungs following systemic administration. Thus, biodegradable polymeric nanoparticles, via photo-crosslinking, demonstrate extended colloidal stability and efficient delivery of RNA therapeutics under physiological conditions, and thereby potentially advance systemic delivery technologies for nucleic acid-based therapeutics.

### 1.2 Overview

Cationic polymers have shown promise as vectors for nucleic acid delivery given their ability to spontaneously self-assemble with anionic siRNA into condensed nanoparticles with efficient payload encapsulation. Poly(beta-amino ester) s (PBAE) s are one class of biodegradable cationic polymers being explored for nucleic acid delivery including for plasmid DNA, and with polymer structural modifications for siRNA. Kozielski et al., 2013; Kozielski et al., 2014; Karlsson et al., 2019; Karlsson et al., 2020. This ability is due to multiple characteristics including their reversible charge, which promotes binding of RNA therapeutics, as well as high buffering capacity for endosomal escape, and their degradability by hydrolysis into nontoxic byproducts under aqueous conditions. Routkevitch et al., 2020; Wilson et al., 2019.

Despite the promising characteristics of PBAEs and cationic nanoparticles in general, positive surface charge when injected often leads to low transfection efficacy following systemic administration due to non-specific interactions with serum proteins and nanoparticle aggregation, resulting in poor colloidal stability and clearance by the mononuclear phagocyte system. Morille et al., 2008; Alexis et al., 2008. To reduce non-specific protein interactions, one promising strategy is charge-shielding of the nanoparticle surface via either coatings, Sizovs et al., 2013, or decationization, Novo et al., 2013.

In addition, lower surface charge generally decreases the risk of toxicity. Karlsson et al., 2018. The most explored surface modification for charge-shielding of nanoparticles is the introduction of poly(ethylene glycol) (PEG) groups via adsorption or conjugation; however, while improving colloidal stability, PEGylation may also impede cellular uptake and endosomal escape, thus decreasing the overall efficacy of the formulation. Hatakeyama et al., 2011. In addition to charge-shielding, crosslinking is another strategy to improve colloidal stability through covalent stabilization of nanostructures, which otherwise rely on electrostatic interactions, to form robust functional nanomaterials. O'reilly et al., 2006. Photopolymerization for crosslinking to form macroscopic biomaterials has already been proven to allow ease of tuning material properties and scalable fabrication. Peppas et al., 2006; Hennink and van Nostrum, 2012. Thus, photo-crosslinking is a promising strategy for improving the functionality of nanoscale biomaterials as delivery vehicles.

Bioreducibility, achieved by the incorporation of disulfide linkages into the polymer structure, enables polymers and polymeric nanoparticles to degrade quickly in a triggered, environmentally-sensitive way, such as in the reducing environment of the cytosol. Karlsson et al., 2019. In some embodiments, the presently disclosed subject matter provides photo-crosslinked bioreducible nanoparticles (XbNPs) based on PBAE for nucleic acid-based therapeutics. Addition of a photo-crosslinking polymer to bioreducible variants of PBAE structures yielded fully bioreducible particles with neutral surface charge, decreased non-specific protein binding, and improved colloidal stability. Although UV-initiated polymerization has previously been demonstrated as a strategy to incorporate acrylate-containing molecules with added functionality for nanoscale delivery systems, Tzeng and Lavik, 2010; Fisher and Peppas, 2009; Fisher et al., 2009; Forbes and Peppas, 2014, this is, to our knowledge, the first reported photo-crosslinked polymeric nanoparticle platform using a PBAE carrier. Critically, it is the first reported photo-crosslinked bioreducible polymeric nanoparticle platform using a bioreducible PBAE carrier. Importantly, PBAE-based bioreducible nanoparticles show promise as RNA delivery vectors from a safety perspective, as well, as their bioreducible nature enables environmentally-triggered degradation is the reducing environment of the cytosol and makes them attractive for nucleic acid delivery. Luly et al., 2020.

The presently disclosed XbNP platform has the potential to address a variety of challenges facing nanoparticle gene delivery, as the XbNPs demonstrate improved stability in serum, improved cellular uptake, and tissue-mediated delivery to extrahepatic tissues compared to their non-crosslinked counterparts. The presently disclosed XbNPs were used to knock down a reporter gene in various patient-derived cancer cell lines and in murine glioblastoma and melanoma cell lines, as well as in a metastatic melanoma model following systemic administration in vivo.

### 2. Results and Discussion

### 2.1. Synthesis, Design, and Nanoparticle Characteristics

### 2.1.1. Synthesis of Bioreducible Polymers and Photo-Crosslinking of Polymeric Nanoparticles

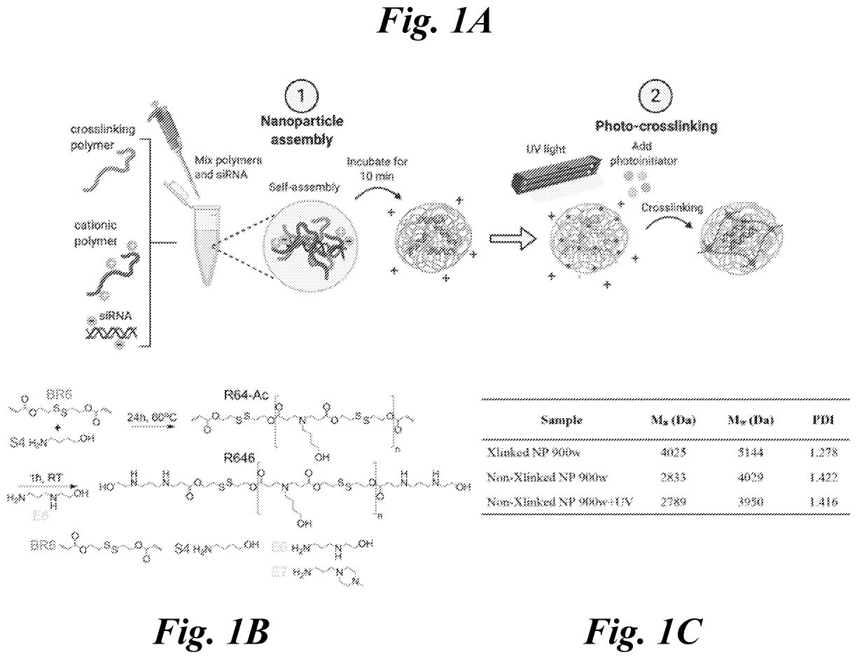

Self-assembly was used to form well-defined nanoparticles. Whitesides and Mathias, 1991; Zhang, 2003. The amine-terminated cationic polymer PBAE electrostatically binds anionic siRNA and spontaneously forms nanoscale particles under mildly acidic aqueous conditions (pH=5.0). In addition to a cationic polymer for siRNA binding, an acrylate-terminated PBAE was added as the crosslinking polymer (FIG. 1A). After self-assembly into nanoparticles, a radical photoinitiator (IRGACURER 2959; Irg) was added and UV light was applied to form crosslinks between the acrylate groups. Both the amine-terminated polymer and the acrylate-terminated polymer for crosslinking contain disulfide bonds in their backbone structures to enable cytosolic glutathione (GSH)-triggered siRNA release. The acrylate-terminated crosslinking polymer R64-Ac and the cationic endcap polymers R646/R647 were synthesized using one- or two-step Michael Addition reactions, respectively (FIG. 1B). Gel permeation chromatography (GPC) was used to measure the polymer molecular weights and to verify crosslinking (FIG. 1C). The molecular weight after nanoparticle crosslinking was 42.1% or 27.7% (by Mₙ or M_{w}, respectively) greater than that of the non-crosslinked formulation. This increase in molecular weight corresponds to formation of covalent bonds between the acrylate-terminated crosslinking polymers (R64-Ac); and the observed increase is expected, given that these R64-Ac crosslinking polymers make up 25% of the initial polymer population. In the absence of the photoinitiator, there is no difference in polymer molecular weight with or without UV exposure, indicating a lack of crosslink formation. The short UV exposure time of 1 min is sufficient to form crosslinks and does not cause any measurable degradation of the polymeric nanocarrier. This characteristic is a major advantage when using photo-crosslinking compared to other crosslinking strategies, since it proceeds quickly under mild reaction conditions.

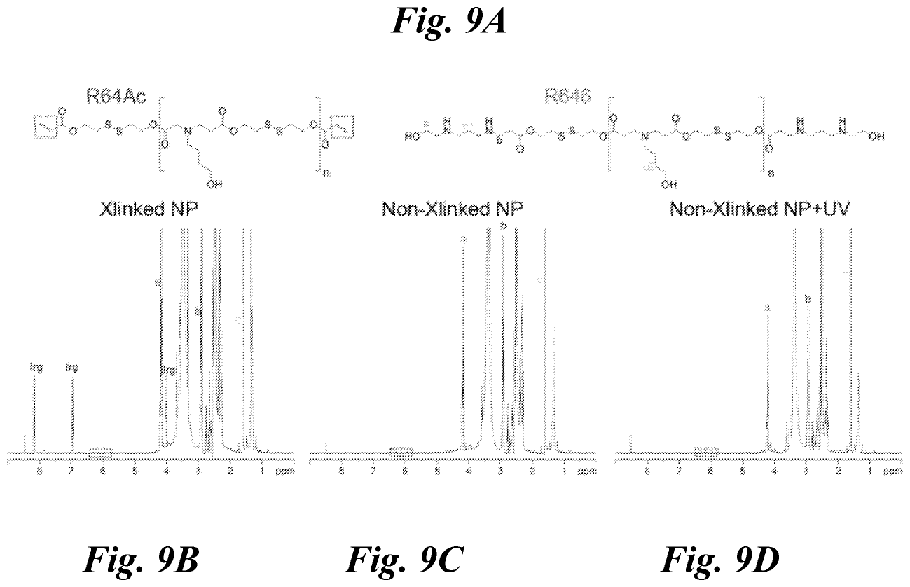

¹H NMR also was carried out to assess the degree to which acrylate groups were crosslinked. The acrylate peaks were integrated and normalized to peaks corresponding to protons in the backbone of the polymer structure for both crosslinked and non-crosslinked nanoparticle formulations (FIG. 9). The peak intensity of the acrylate peaks decreased 79.1±0.3%, indicating that most of the acrylate groups formed crosslinks. It is possible that not all acrylate groups formed crosslinks due to a lack of other acrylate groups in close proximity with which to react; however, the radical photo-crosslinking efficiency seen in the XbNP system is comparable to that seen in similar photo-polymerization reactions by other groups. Fisher et al., 2009.

Due to the efficient crosslinking reaction of acrylate groups, the density of covalent crosslinks in the particle can easily be adjusted by altering the ratio between the crosslinking and the amine-terminated polymer. Moreover, to reduce the risk that the added covalent crosslinked network would impede timely intracellular release, disulfide bonds were incorporated into the backbone of both the acrylate- and amine-terminated polymers to facilitate triggered cytosolic release. Cargo release can easily be modulated through incorporation of bioreducible groups and by using PBAE structures with altered hydrophilicity to tune the rate of hydrolytic degradation. The ease of PBAE synthesis is also beneficial for forming polymer structures with diverse chemical properties, which has allowed high-throughput testing of combinatorial libraries of polymers to identify PBAEs structures for efficient transfection of various cell-types. Anderson et al., 2003; Green et al., 2008.

Further, modulations of the PBAE nanocarriers also can be made for cell-type specificity of nucleic acid therapeutics. Sunshine et al., 2009. These nanoparticle designs have demonstrated the ability to efficiently deliver genes in vivo after local administration. Mangraviti et al., 2015; Lopez-Bertoni et al., 2018. These previous formulations, however, have had limited success for systemic administration mainly due to insufficient stability in the presence of anionic serum proteins that readily dissociate the formulation prior to reaching the targeted site. Wang et al., 2019. Thus, the presently disclosed XbNP platform could potentially improve extracellular colloidal stability by the addition of covalent bonds, thus improving their likelihood of therapeutic success when administrated systemically. This crosslinked design also can be applied to other cationic polymeric nanoparticles that are formed by self-assembly principles to improve their functionality and stability under physiological conditions to facilitate systemic delivery of nucleic acid therapeutics.

### 2.1.2. Physical Nanoparticle Properties: Photo-Crosslinking for Shielded Surface Charge and Reduced Protein Adsorption

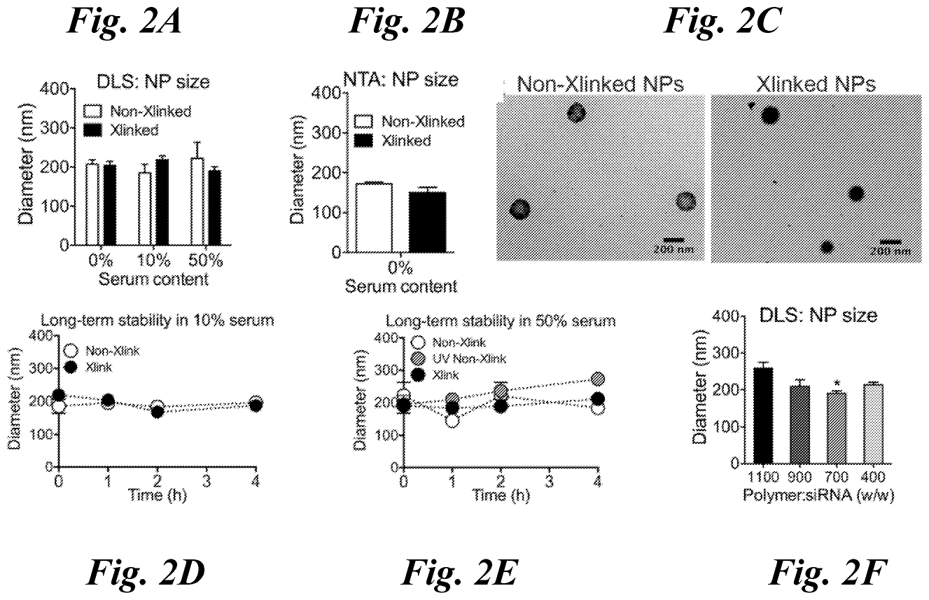

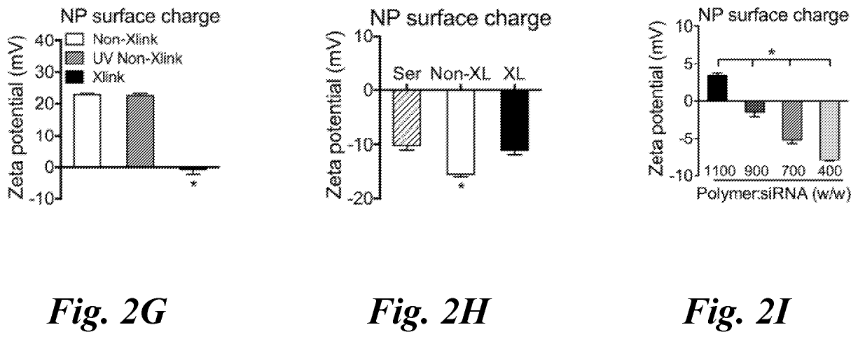

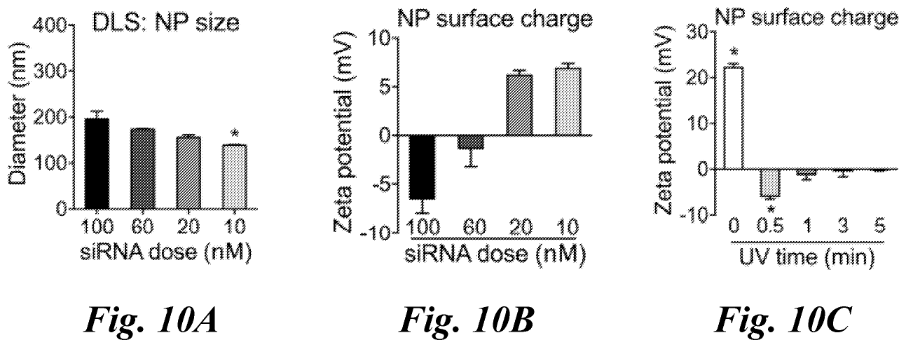

Dynamic light scattering (DLS), nanoparticle tracking analysis (NTA), and transmission electron microscopy (TEM) were used to assess nanoparticle size and surface charge. No differences were observed in nanoparticle size between crosslinked and non-crosslinked particles, their size was 204±11 nm and 207±12 nm, respectively, measured by DLS at the 60 nM siRNA dose and 900 weight ratio polymer to siRNA (w/w) in PBS (FIG. 2A). Nor did incubation in 10-50% serum influence the size when compared to incubation in PBS (0% serum). In all conditions and by all analytical techniques used, the nanoparticle size was similar both with and without crosslinking (FIG. 2A-FIG. 2C). The nanoparticle size decreased slightly with lower siRNA dose and w/w ratios of polymer:siRNA (FIG. 10A, FIG. 2F). Further, both the crosslinked and non-crosslinked nanoparticle formulations demonstrated stability in the presence of serum (10% and 50%) until the endpoint of 4 h (FIG. 2D-FIG. 2E). It is promising that the nanoparticles remain approximately 200 nm in size over time in presence of serum, as particles between 70 and 200 nm exhibit prolonged circulation. Goldberg et al., 2007.

While nanoparticle sizes were no different between the crosslinked and the non-crosslinked formulations, zeta potential measurements demonstrated that crosslinking reduced the surface charge from ±22.9±0.3 mV for the non-crosslinked nanoparticles to being neutral (−0.8±1.5 mV) after crosslinking (FIG. 2G). This shielding of the cationic charge is beneficial for promoting colloidal nanoparticle stability in the bloodstream, as interactions with anionic serum proteins may lead to nanoparticle dissociation and loss of encapsulated siRNA during circulation. Yin et al., 2014; Morille et al., 2008. Poor colloidal stability may influence experimental outcomes, both in vitro and in vivo, by affecting mechanisms such as cellular uptake and increasing overall toxicity, Moore et al., 2015, and is a leading reason why cationic nanoparticles have not been sufficiently effective at the delivery of RNA therapeutics upon systemic administration. Blanco et al., 2015.

Moreover, nanoparticle formulations should avoid adsorption of serum opsonins to prevent recognition and clearance by the mononuclear phagocyte system. Alexis et al., 2008. Thus, nanoparticles that interact less with the biological environment are desirable for prolonged circulation to reach targeted tissues. Some cationic nanocarriers require surface modifications to minimize nonspecific interactions, with the most common approach being PEGylation of the nanoparticle surface for steric shielding. Suk et al., 2016. The incorporation of PEG, however, may reduce the degree of intracellular delivery of RNA therapeutics, thus these systems may require PEG de-shielding for successful intracellular trafficking. Hatakeyama et al., 2011; Suk et al., 2016; Li and Huang, 2010.

Decationization is another strategy to improve the circulation time when using cationic polymeric nanocarriers, in which the polymer undergoes hydrolysis of cationic groups to form neutral or negatively charged nanoparticles prior to administration. Novo et al., 2013.

The presently disclosed subject matter demonstrates that photo-crosslinking can shield the surface charge that is otherwise positive due to the cationic polymer, eliminating the need for additional modifications to achieve neutral surface charge. Further, the crosslinked nanoparticles can be tuned to be slightly positive or negative by adjusting the ratio between the cationic polymer and the RNA dose (FIG. 2I). UV exposure times as short as 0.5 min are sufficient for loss of the cationic charge (FIG. 10F). When incubated in serum, the non-crosslinked nanoparticles demonstrated statistically lower surface charge (p=0.0039; n=3) than the serum itself, whereas no difference was observed for the XbNPs (FIG. 2H). This observation indicates that there is higher adsorption of serum proteins to the non-crosslinked nanoparticles, thus conferring anionic charge.

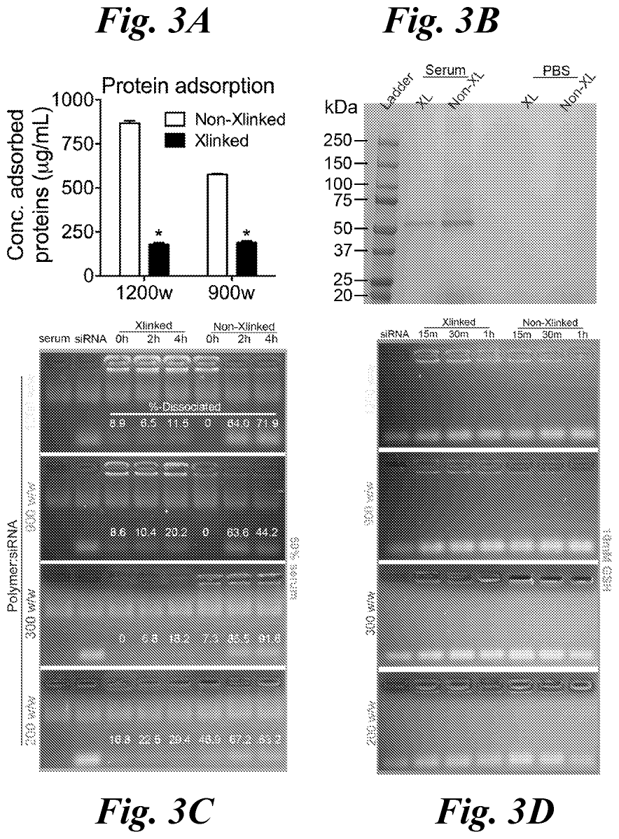

The ability of photo-crosslinking to alter protein adsorption under high serum conditions was first assessed using a bicinchoninic acid (BCA) assay. The amount of protein adsorbed onto both non-crosslinked and crosslinked nanoparticles was compared across two different weight ratios, 1200 w/w and 900 w/w. The protein adsorption was significantly lower for the XbNPs for both the 1200 w/w and 900 w/w formulations (FIG. 3A). This observation is likely due to the decrease in nanoparticle surface charge following photo-crosslinking, thus reducing the ionic interactions between the nanoparticles and anionic serum proteins (FIG. 2G). The decreased interactions with serum proteins may aid in XbNPs' translation as a delivery technology for systemic administration, as the otherwise major limiting hurdle when using cationic polymeric nanocarriers is the competitive binding of polyanions that destabilize the formulation. Wang et al., 2019.

While protein adsorption following intravenous (i.v.) injection results in a protein corona around the NP, which might impede intracellular delivery, adsorption of specific serum proteins may be beneficial for mediating cell- and tissue-specific delivery. Morille et al., 2008; Cedervall et al., 2007; Doctor et al., 2015; Ke et al, 2017; Zhang et al., 2020. The composition of the adsorbed proteins was further examined by running sodium dodecyl sulfate-polyacrylamide gel electrophoresis (SDS-PAGE) on crosslinked and non-crosslinked nanoparticles after incubation in serum and in PBS as control. In the serum condition, the XbNPs displayed fewer bands of lower intensity (FIG. 3B), which supports the finding from the BCA assay that photo-crosslinking lowers the overall protein adsorption. The most distinct band of the adsorbed proteins onto both the crosslinked and non-crosslinked nanoparticles was at 58 kDa, corresponding to albumin, which is one of the most abundant proteins found in serum and has been shown to promote receptor-mediated nanoparticle uptake in cancer cells. Hyun et al., 2018.

This phenomenon is due to increased albumin metabolization by cancer cells to support energy and amino acid consumption associated with rapid cancer-cell proliferation. Fisher et al., 2009; Forbes and Peppas, 2014. It is therefore possible that adsorption of high-molecular-weight proteins shields adsorbed albumin, leading to decreased cellular uptake of the non-crosslinked nanoparticle formulations. The results from the SDS-PAGE assay indicate that non-crosslinked nanoparticles adsorb greater amounts of proteins with a molecular weight above 58 kDa than their XbNP counterpart, suggesting that the crosslinked nanoparticles might promote cellular uptake. The specific interaction with albumin for the XbNPs is interesting since it potentially could be useful for biomimetic targeting and tumor accumulation. Cao et al., 2017.

### 2.1.3. Photo-Crosslinking Promotes Colloidal Nanoparticle Stability

An siRNA gel electrophoresis assay was performed to evaluate the colloidal nanoparticle stability and the encapsulation efficiency over time. Crosslinked and non-crosslinked nanoparticle formulations with weight ratios (w/w) 1200, 900, 300, and 200 were prepared and incubated in 50% serum over 4 h. The dissociation of the nanoparticles, measured by release of the siRNA payload, was markedly lower for the crosslinked particles across all tested formulations both after 2 and 4 h, with the majority of the siRNA dose dissociated from the non-crosslinked formulations at 2 h (FIG. 3C). Thus, photo-crosslinking of the nanoparticle formulations improves the encapsulation efficiency and colloidal stability of the particles under high-serum conditions. The reduced surface charge and corresponding decreased interactions with serum proteins are advantageous as cationic polymeric carriers rely on electrostatic interactions to bind and encapsulated RNA molecules. These cationic polymer/siRNA nanocomplexes and can easily dissociate due to competitive binding of cationic polymer to other anionic biomolecules. Blanco et al., 2015. Competitive binding to anionic proteins in serum is a likely cause of the observed higher degree of nanoparticle dissociation and siRNA release for the non-crosslinked formulations.

A gel electrophoresis assay was carried out after nanoparticle incubation in a cytosolic-mimicking environment (10 mM glutathione) Manickam et al., 2010; Meng et al., 2009, to examine whether the crosslinking would affect the intracellular release kinetics. The high concentration of glutathione in the cytosol readily cleaves disulfide bonds upon entry to the cytosol, leading to release of encapsulated molecules. Peppas et al., 2006; Hennink and van Nostrum, 2012; Hyun et al., 2018.

Across the tested formulations using 1200, 900, 300, and 200 w/w, no delay in the siRNA release in the cytosolic-mimicking environment was observed for the crosslinked nanoparticles compared to the non-crosslinked nanoparticles (FIG. 3D). Both the crosslinked and non-crosslinked particles exhibited triggered siRNA release in the reducing environment within 15 min.

This observation is due to the presence of disulfide bonds in the structure of both of the polymers used for nanoparticle formation. The similar release kinetics between non-crosslinked and crosslinked nanoparticles indicate that crosslinking does not interfere with rapid intracellular release of RNA therapeutics delivered by these bioreducible polymeric carriers. The incorporation of stimulus-responsive crosslinks in the nanoparticle formulations that rely on electrostatic interactions for improved colloidal stability in the blood stream and in extracellular spaces holds great promise for successful translation. Karlsson et al., 2018; Mura et al., 2013.

A strategy of high interest is the use of environmentally-triggered crosslinks containing disulfide bonds, since these materials provide stability during extracellular circulation while being readily cleaved in cytosol due to the high concentration of glutathione leading to a quick release of the payload. Meng et al., 2009; Kakizawa et al., 1999; Son et al., 2012. In the presently disclosed XbNP design, both the acrylate-terminated and the amine-terminated polymers contained disulfide bonds for triggered cytosolic release while preventing nanoparticle dissociation prior to being internalized into targeted cells.

2.2. In Vitro siRNA Delivery: Photo-Crosslinked Nanoparticles as a Platform for Intracellular Delivery to Cancer Cells
2.2.1. Crosslinked Nanoparticles Mediate siRNA Delivery in Patient-Derived Cancer Cells Under High-Serum Conditions

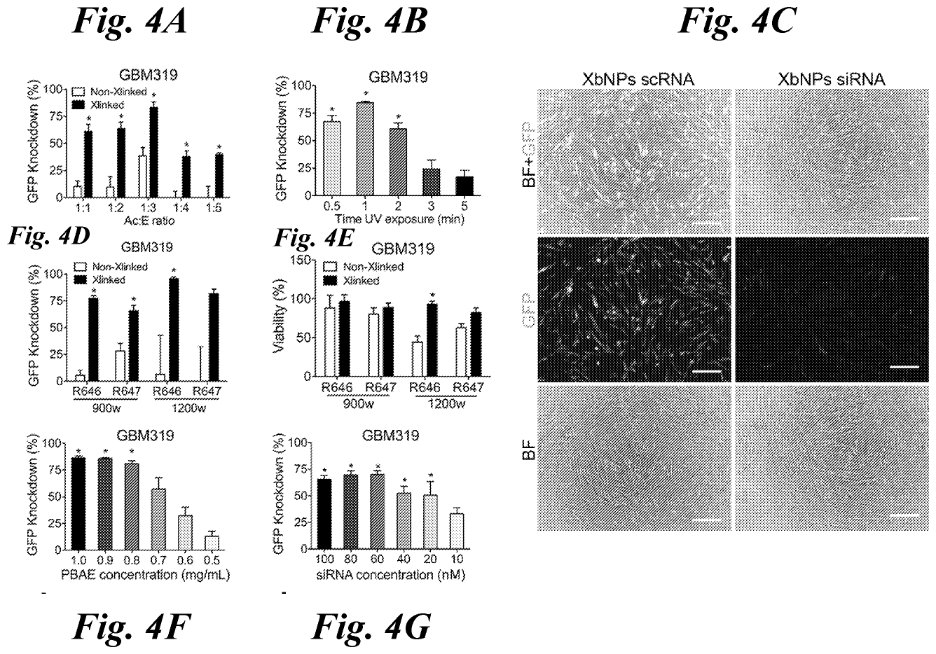

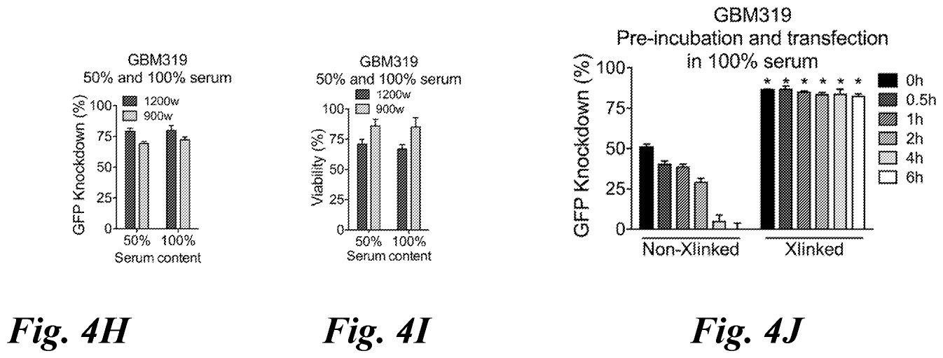

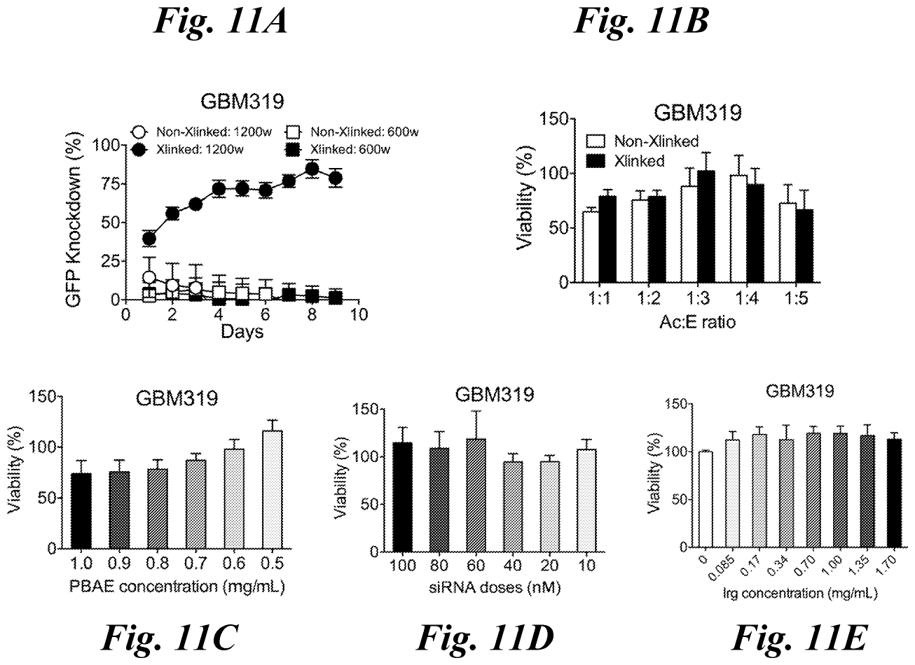

The siRNA delivery efficacy of the engineered XbNP formulations was evaluated under high-serum conditions (50% serum) to better mimic the environment of the bloodstream after systemic delivery, with patient-derived glioblastoma cells (GBM319) as the first cancer cells tested. To modulate the degree of crosslinking, the ratio between the acrylate polymer (R64Ac) and end-capped polymer (R646) was varied, and nanoparticles were formulated with and without photo-crosslinking. For all the ratios tested, the crosslinked formulations provided superior siRNA-mediated GFP knockdown in the GBM319 human brain cancer cells compared to the matched non-crosslinked formulations with an optimized mass ratio of 1:3 (R64Ac to R646) causing 83%+5% silencing of GFP expression (FIG. 4A). The duration of UV exposure needed for photo-crosslinking was then explored and it was observed that only 0.5 min of UV exposure was required to achieve efficient transfection in high-serum conditions, with no significant difference measured for UV exposures between 0.5 and 2 min (FIG. 4B). When UV exposure lasted for 3 min or longer, siRNA-mediated knockdown decreased significantly, likely due to degradation of the polymer. Nanoparticle formulations with varying weight ratios (w/w) between the polymer and siRNA dose also were compared, as were those containing amine-terminated polymers with different endcaps (termed R646 or R647). For all tested formulations, the crosslinked nanoparticles outperformed their non-crosslinked counterparts, and the formulations with R646 led to the greatest transfection of up to 96%+2% siRNA-mediated GFP knockdown (FIG. 4C-FIG. 4D, FIG. 11A). This great efficacy observed under high-serum conditions shows the tremendous potential of XbNPs to be used as a nanoparticle platform for systemic siRNA delivery. The presence of serum in culture media generally interferes with in vitro transfection. Polyethylenimine (PEI), for instance, is a commonly used nanocarrier with efficient transfection capability in serum-free media; however, the addition of just 10% serum drastically decreases its efficacy. Liu et al., 2016.

Not only did photo-crosslinking improve the transfection efficacy of the nanoparticles, but it also reduced toxicity. At the highest tested polymer concentration prepared, the 1200 w/w ratio, a 50% decrease in viability was observed for the R646-based non-crosslinked formulation, whereas the photo-crosslinked formulation showed minimal toxicity (FIG. 4E). The reason for the reduced toxicity is likely due to the shielded surface charge after photo-crosslinking, as cationic nanoparticles have been shown to exhibit increased toxicity compared to neutral or anionic particles. Sukhanova et al., 2018.

Further, the polymer concentrations and siRNA doses required for high transfection efficacy in high serum conditions were evaluated. For the polymer, a concentration of 0.8 mg/mL or higher is required to achieve greater than 75% siRNA-mediated knockdown (FIG. 4F). For the siRNA dose, there was no statistical difference in the transfection between the doses of 20 and 100 nM, which shows that the photo-crosslinked nanocarrier provided highly potent siRNA delivery to patient-derived glioblastoma cells (FIG. 4G) at low dose. Low toxicity was observed across all the tested crosslinked nanoparticle formulations (FIG. 11B-FIG. 11D). No cellular toxicity was observed for the photo-crosslinker Irg at the transfection concentration of 0.17 mg/mL and the concentration can even be increased at least by a magnitude to 1.7 mg/mL without any toxicity issues (FIG. 11e). Williams et al., 2005, demonstrated in their study that Irg (Irgacure 2959) is well tolerated by many cell types and species.

2.2.2. Long-Term Colloidal Nanoparticle Stability Following Pre-Incubation in Serum without Loss in Efficacy

The efficacy of the engineered nanoparticles in complete (100%) serum conditions was evaluated to model systemic administration. The XbNPs demonstrated the same degree of siRNA-mediated knockdown in patient-derived glioblastoma cells in complete serum as in 50% serum condition with low toxicity (FIG. 4H-FIG. 4I). To examine the long-term colloidal nanoparticle stability in complete serum, the XbNPs and non-crosslinked particles were pre-incubated in 100% serum at 37° C. for up to 6 h prior to transfection experiments in complete serum. The degree of siRNA-mediated knockdown decreased as pre-incubation time increased for the non-crosslinked nanoparticles, whereas knockdown with the XbNPs was unaffected: even after 6 h of pre-incubation in complete serum, XbNPs caused 82%+2% GFP knockdown (FIG. 4J). Together, the long-term stability and high transfection efficacy under high-serum conditions show the promise of the XbNP platform for systemic siRNA delivery.

### 2.2.3. Cellular Uptake and Endosomal Escape of Engineered Nanoparticle Formulations

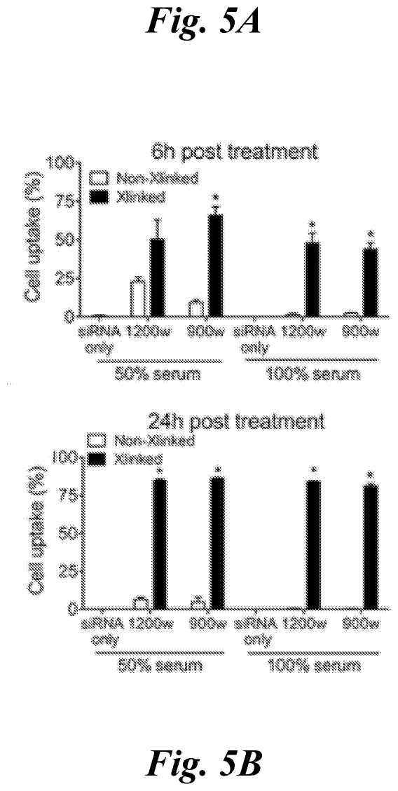

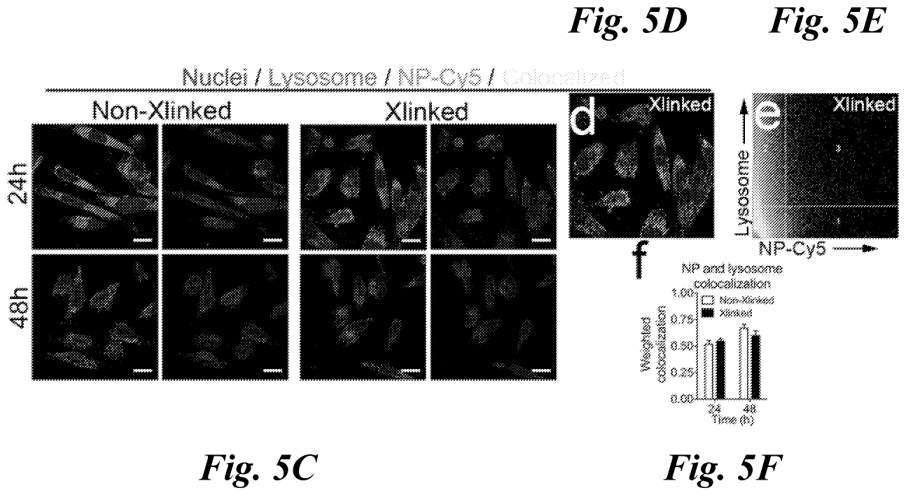

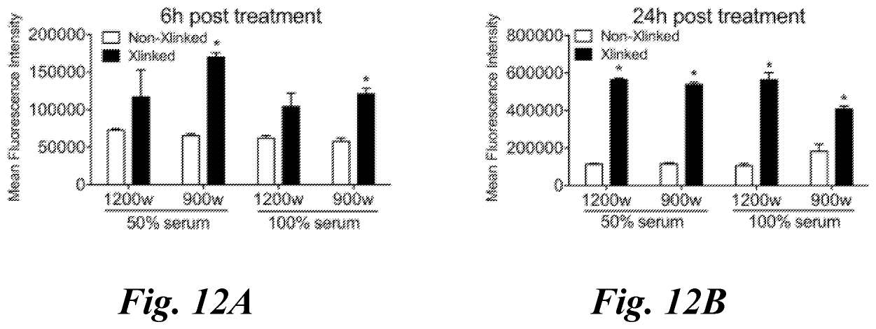

To elucidate the mechanisms of the improved siRNA delivery efficacy for the XbNPs, cellular uptake and endosomal escape were evaluated in patient-derived glioblastoma cells at high serum conditions. In these experiments, Cy5-siRNA was used, and cellular uptake was initially compared at different time-points post-transfection. After both 6 h and 24 h post-treatment and in both 50% and 100% serum, the XbNPs were taken up by cells more efficiently than non-crosslinked nanoparticles (FIG. 5A-FIG. 5B, FIG. 12). To study endosomal escape, confocal microscopy was used to visualize the nuclei and lysosomes, along with the Cy5-siRNA in the nanoparticle formulations (FIG. 5C). Endosomal escape was evaluated by quantifying the colocalization of the lysosomes and Cy5-nanoparticles, with lower colocalization corresponding to effective endosomal escape. The results showed no differences between the crosslinked and non-crosslinked nanoparticle formulations, demonstrating that the shielded charge following photo-crosslinking does not significantly affect endosomal escape (FIG. 5D-FIG. 5F). Without wishing to be bound to any one particular theory, the mechanism through which XbNPs facilitate enhanced siRNA-mediated knockdown is thought to be via improved cellular uptake. The XbNPs with improved encapsulation of siRNA, prolonged particle stability in pure serum (FIG. 3C), and reduced adsorption of high-molecular-weight proteins (such as immunoglobulins) (FIG. 3B) enhance cellular uptake.

2.2.4. Nanoparticle Platform for siRNA Delivery to Various Glioma Cells

The XbNPs were evaluated for their potential to serve as a platform for efficient siRNA delivery to other glioblastoma cell lines. This characteristic is of importance because brain tumors are heterogeneous; Soeda et al., 2015, hence, robust delivery to various brain cancer cells is required for effective treatment of glioma patients. The nanoparticle formulations were tested in GBM1A, a patient-derived glioblastoma cell line with high stemness. Tilghman et al., 2014. Stem-like glioma cells are extremely evasive and resistant when it comes to radiation therapies, Bao et al., 2006, and contribute greatly to disease progression. Park and Rich, 2009.

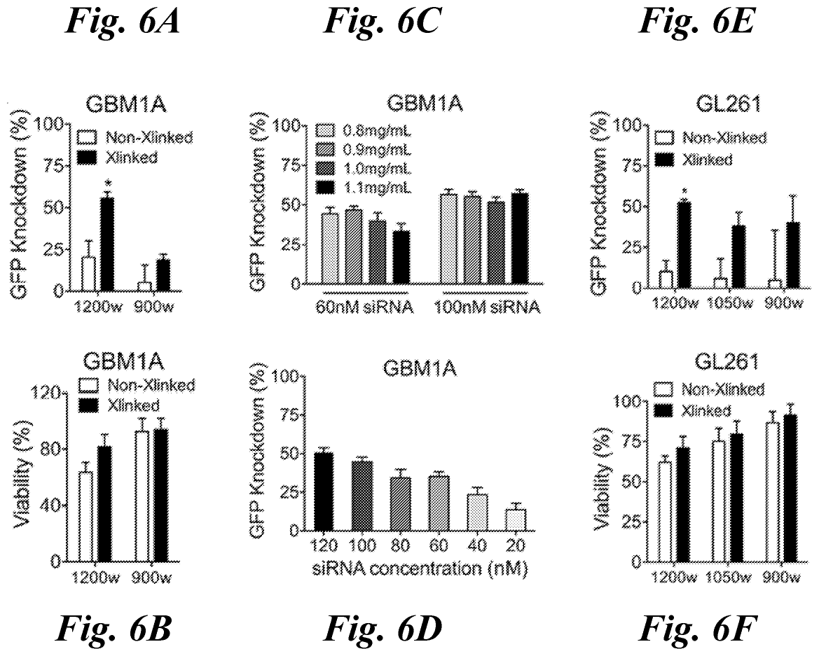

Novel therapeutics for glioblastoma must be able to address glioma cells broadly, including those with stem-like properties, to control disease progression. In GBM1A, XbNPs at 1200 w/w achieved >50% siRNA-mediated GFP knockdown while still having low toxicity (FIG. 6A-6B). Over a range of siRNA dose from 20 nM to 120 nM and polymer doses from 0.8-1.1 mg/mL, corresponding to 500-1200 w/w, knockdown was observed to be approximately 10-50% (FIG. 6C-FIG. 6D), with knockdown in GBM1A stem-like brain cancer cells especially sensitive to siRNA dose.

The XbNPs also were evaluated in GL261 cells, a commonly used murine glioma model. As in the GBM1A cells, the XbNP formulation of 1200 w/w caused >50% GFP knockdown with low cytotoxicity (FIG. 6E-FIG. 6F). Together, as observed in the patient-derived glioblastoma cell line GBM319, the XbNP formulations also outperformed the non-crosslinked formulations in GBM1A and GL261 cells in terms of siRNA-mediated knockdown. The results suggest that the XbNPs could potentially serve as a next-generation nanoparticle platform for siRNA-based therapeutics for glioma. Their ability to transfect diverse glioma cells is crucial given the intrinsic heterogeneity of glioblastoma, Soeda et al., 2015, and future genetic therapeutics that address the heterogenous cell population may lead to improved outcomes for glioma.

2.2.5. Photo-Crosslinked Nanoparticles for siRNA Delivery to Melanoma Cells

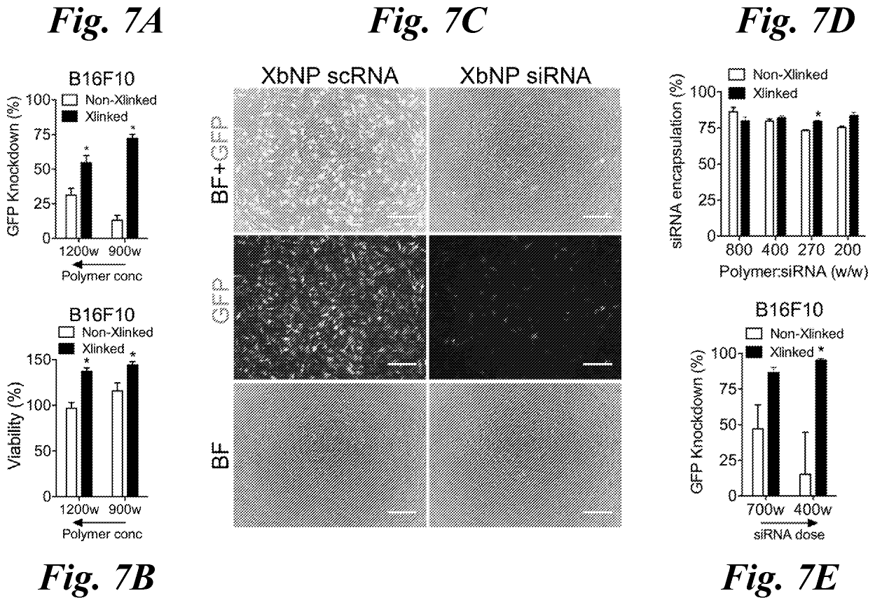

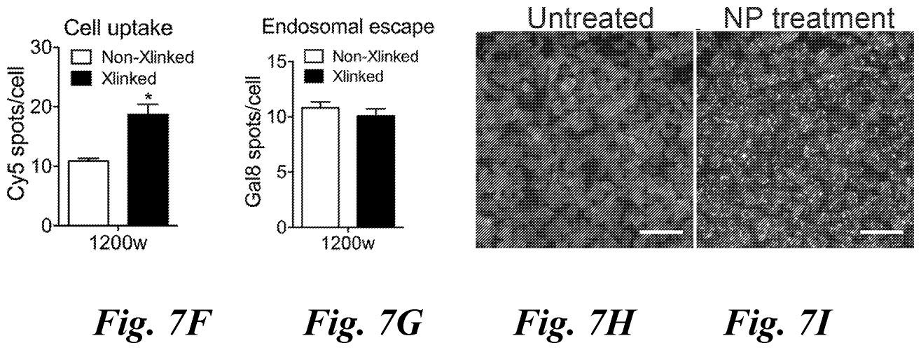

The XbNPs' ability to provide siRNA-mediated knockdown in murine melanoma cells (B16F10) in high-serum conditions (50% serum) was further evaluated. The XbNPs outperformed their non-crosslinked counterparts in terms of both increased knockdown efficacy and reduced toxicity (FIG. 7A-FIG. 7C). A critical property of nanocarriers to enable potent systemic delivery of siRNA is high and stable encapsulation of the siRNA therapeutics. To assess the encapsulation efficiency, a RiboGreen™ RNA assay was performed, in which the siRNA dose was increased while keeping polymer concentration constant to modulate the w/w of nanoparticle formulations. Across all the tested nanoparticle formulations (200-800 w/w), efficient siRNA encapsulation was observed (FIG. 7D), with the XbNPs at 270 w/w showing statistically higher siRNA encapsulation than the non-crosslinked formulation. Higher siRNA payload promotes transfection efficacy, as the 400 w/w formulation provided almost complete siRNA-mediated GFP knockdown (FIG. 7E).

### 2.2.6. Gal8-Assay Demonstrates Improved Cellular Uptake in Melanoma Cells for Crosslinked Nanoparticles

Critical hurdles for successful intracellular delivery of nucleic acid-based therapeutics involve cellular uptake and endosomal escape. Nanoparticle endocytosis must be followed by endosomal escape for successful delivery; otherwise, endosomal entrapment renders the nanoparticle and its cargo useless as it is degraded via the endo/lysosomal pathway. Smith et al., 2019.

For polymeric nanoparticles with titratable amine groups, endosomal escape occurs in part as the internalized particle buffers pH changes in the endocytic vesicle, ultimately leading to an increase in osmotic pressure and subsequent vesicle rupture and nanoparticle release. Vermeulen et al., 2018.

This mechanism, termed the “proton sponge effect”, has been the subject of debate among groups studying nanoparticle-based drug and gene delivery. Vermeulen et al., 2018. Although elucidation of the exact mechanisms underlying endosomal escape of PBAEs is still under active investigation, a recently developed assay utilizing Gal8 to visualize endosomal disruption allows the quantitative assessment of nanoparticle endosomal escape in vitro. Kilchrist et al., 2019. This method was used to evaluate cellular uptake and endosomal disruption in B16F10 cells, in which the XbNP and non-crosslinked formulations contained Cy5-siRNA and the B16F10 cells expressed a Gal8-GFP fusion protein. In the assessment of cellular uptake quantifying Cy5-spots/cell, statistically higher uptake was demonstrated for the XbNPs compared to non-crosslinked nanoparticles (FIG. 7F). To assess endosomal escape, Gal8-GFP spots/cell were quantified, corresponding to the number of endosomal disruption events. Kilchrist et al., 2019. Endosomal escape was not different between the XbNPs and non-crosslinked nanoparticles (FIG. 7G). Representative microscopy images from the Gal8-assay comparing untreated and nanoparticle-treated cells (FIG. 7H-FIG. 7I) clearly demonstrate how efficient the engineered PBAE nanocarrier is at facilitating endosomal disruption for intracellular siRNA delivery to the cytoplasm. This efficiency demonstrates that the mechanism for improved siRNA delivery efficacy of the XbNPs in B16F10 cells is improved cellular uptake of siRNA, which is in line with the mechanistic results found in patient-derived glioblastoma cells. Additionally, the XbNP formulation contains both secondary- and tertiary amines that undergo protonation at the lower pH of the endosomal compartment leading to osmotic pressure, which causes endosomal disruption. The results indicate that, despite the surface charge-shielding due to photo-crosslinking, amines remain accessible for buffering in the XbNP formulation, thus leaving the desirable endosomal escape efficiency unchanged.

2.3. Photo-Crosslinked Nanoparticles for Systemic siRNA Delivery to Tumors In Vivo

### 2.3.1. Tissue-Mediated Nanoparticle Delivery

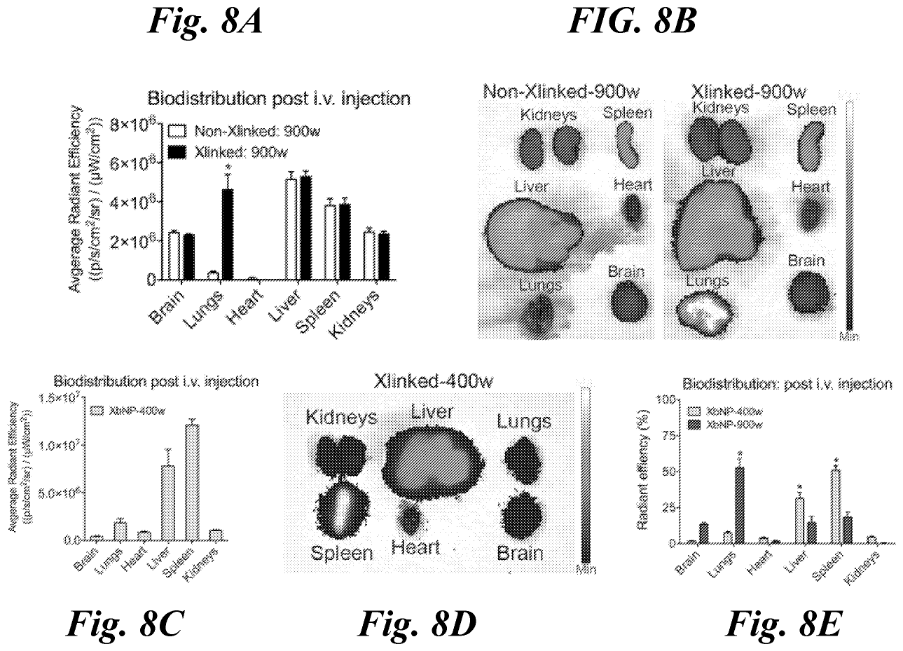

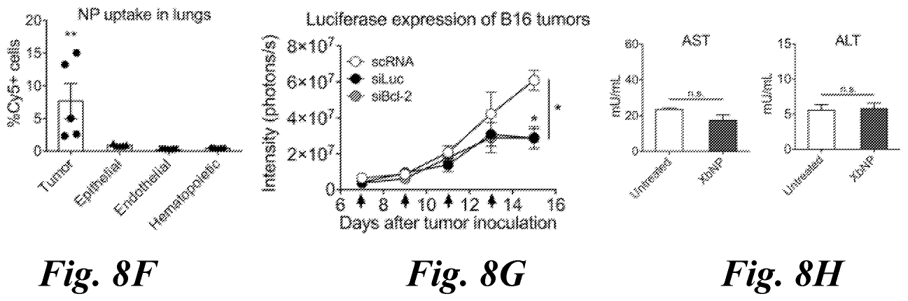

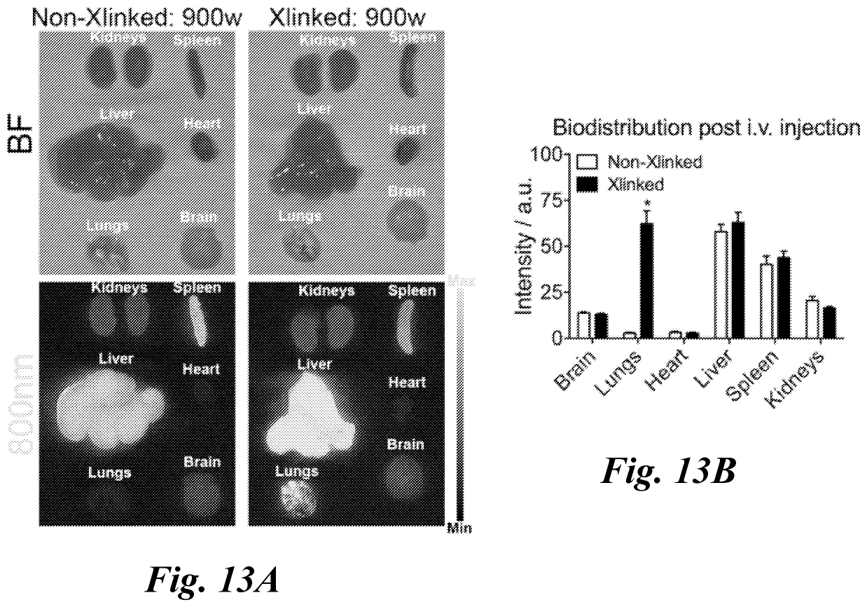

The XbNPs were evaluated for systemic siRNA delivery following i.v. injection. XbNPs containing IR-labeled siRNA were initially used to evaluate biodistribution after systemic administration. This study demonstrated that photo-crosslinking PBAE nanoparticles improved targeting to the lungs (FIG. 8A-FIG. 8B and FIG. 13).

In addition, both XbNPs and non-crosslinked nanoparticles facilitated siRNA delivery to the brain to some degree. This finding is in agreement with a recent study showing that non-crosslinked PBAE nanoparticles facilitated active transport across a biomimetic in vitro assay of the BBB endothelium and delivery to the brain in vivo following systemic administration. Karlsson et al., 2019. This result might be due in part to the adsorption of albumin shown for both XbNPs and non-crosslinked PBAE nanoparticles (FIG. 3B). Lin et al., 2016, demonstrated that their albumin-based nanoparticles facilitated BBB crossing via mechanisms of SPARC and gp60-mediated transport. Moreover, differences in cumulative fluorescent intensity observed between crosslinked and non-crosslinked formulations are most likely due to the nanoparticles that did not extravasate and accumulate in organs, but instead were excreted in urine and stool.

To broaden the potential of nanomedicine carrying RNA therapeutics, there is a need for nanocarriers capable of extrahepatic delivery. The recent success of the lipid nanoparticle formulation Onpattro that was FDA-approved in 2018 demonstrated liver-targeted siRNA delivery for the treatment of polyneuropathies. Akinc et al., 2019. Based on learnings from Onpattro's clinical success, key features required for clinical translation are low surface charge and efficient siRNA encapsulation, both of which are facilitated by photo-crosslinking in this study. Despite early translational success for RNA-based therapies, the challenge remains to develop nanoparticle designs for delivery targeted to tissues beyond the liver. The Onpattro lipid nanoparticles facilitate adsorption of apolipoprotein E (ApoE; 34 kDa) following i.v. administration, leading to delivery to hepatocytes. Akinc et al., 2019.

In contrast, the presently disclosed XbNPs have specific affinity to albumin (58 kDa; FIG. 3B), which facilitates different biodistribution. A key factor that likely correlates with the protein adsorption is the nanoparticle surface charge, which can dictate tissue-targeting. Cheng et al. developed a lipid nanoparticle system termed Selective ORgan Targeting (SORT), in which they modulated the lipid composition of nanoparticles and thereby altered the surface charge through which they were able to achieve tissue-specific mRNA delivery. Cheng et al., 2020.

They demonstrated lung-targeted delivery of their neutrally charged lipid nanoparticle formulation, which is consistent with the current findings. Through slight changes of the surface charge via tuning of the lipid composition, the tissue specificity of the SORT nanoparticles could be tailored. In a non-lipid nanocarrier system, it is demonstrated for XbNPs that altered ratios between polymer and siRNA enabled differential tissue targeting. XbNPs using 400 w/w ratio facilitated preferential organ-delivery to the spleen (FIG. 8C-FIG. 8D). When comparing the radiant efficiency of each organ to all analyzed organs for each formulation, the XbNPs using 900 w/w facilitated statistical higher delivery to the lungs and the XbNPs using 400 w/w facilitated preferential delivery to the spleen (FIG. 8E). The differential tissue targeting observed is likely due to difference in surface charge that can be easily tuned by altering the amount of polymer mixed with siRNA during the self-assembly step of the XbNP formulation (FIG. 2I).

2.3.2. Preferential Uptake in Cancer Cells and siRNA-Mediated Knockdown In Vivo

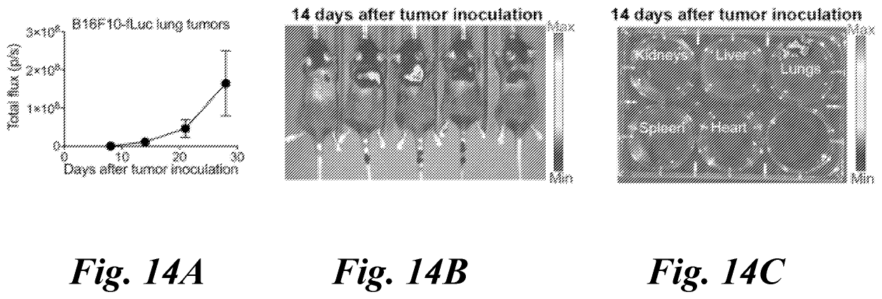

Melanoma cells are highly metastatic and commonly metastasize to the lungs. Zbytek et al., 2008. To establish an in vivo model to approximate melanoma metastasis to the lungs, melanoma cells that expressed luciferase and tdTomato (B16F10-Luc=tdTomato*) were injected i.v. IVIS imaging of the bioluminescence showed that the melanoma cells colonized and formed tumors in the lungs and grew exponentially over time (FIG. 14).

A similar model was established by creating B16F10 tumors expressing GFPd2 to analyze nanoparticle uptake in different cell types of the lungs. XbNPs containing Cy5-siRNA were administered i.v. and the lungs were harvested after 18 hours. Nanoparticle uptake by specific cell populations was assessed by flow cytometry, measuring internalization in cancer cells, epithelial cells (CD31⁺), endothelial cells (CD326⁺), and hematopoietic cells (CD45⁺). The nanoparticle uptake by the melanoma cells was statistically higher compared to all other phenotypes (FIG. 8F; p=0.0033; n=5), demonstrating that the XbNPs selectively targeted cancer cells over other cell types in the lungs. Preferential delivery to cancer cells has been observed for certain PBAE structures in previous in vitro studies. Sunshine et al., 2009, explored DNA delivery using a PBAE nanocarrier in a wide-range of cell types and showed that modulations of the small-molecular endcaps strongly influence cell-specificity.

Other studies using PBAEs as vectors for either DNA or siRNA therapeutics have demonstrated preferential delivery to cancer cells over healthy cells as a result of modulation of the polymer structure. Kozielski et al., 2014; Zamboni et al., 2017; Tzeng et al., 2013; Tzeng et al., 2011. These in vitro transfection studies were all performed in low-serum conditions, allowing the intrinsic properties of PBAEs to facilitate preferential delivery. In the case of XbNPs, the decreased non-specific interactions with serum proteins after administration into the bloodstream suggest that PBAEs can enable preferential delivery to cancer cells even after i.v. administration. In addition, the specific protein interaction of albumin with the nanoparticle surface after photo-crosslinking may serve as another mechanism for tumor targeting, since aggressive cancer cells use albumin as an essential source of energy during their outgrowth. Merlot et al., 2014; Dennis et al., 2007.

For instance, Cao et al., 2017, demonstrated for their nanoparticle system that pre-decoration with albumin prior to administration led to tumor-targeted delivery in metastatic breast cancer model. In a subsequent in vivo experiment, B16F10-Luc⁺ cells were injected and metastasis-like lesions were allowed to form over 7 days prior to treatment. XbNPs carrying either siRNA targeting the luciferase expression, or Bcl-2 as a therapeutic, or scRNA (control) were administered repeatedly via i.v. injections at day 7, 9, 11, and 13 after tumor inoculation. Bcl-2 is an anti-appoptotic protein upregulated in malignant cells; Warren et al., 2019, accordingly, silencing of Bcl-2 expression has been shown to induce apoptosis in malignant melanoma both in pre-clinical and clinical studies. Zbytek et al., 2008; Zhou et al., 2017; Bedikian et al., 2006.

Thus, siRNA targeting luciferase (siLuc) was utilized as the bioluminescence readout gene target and Bcl-2 (siBcl-2) as therapeutic gene target. The bioluminescence from the melanoma in the lungs was monitored over time, which demonstrated knockdown both for XbNPs carrying siLuc and siBcl-2, showing that the XbNP enabled potent systemic siRNA delivery to the melanoma cells (FIG. 8G; p<0.005; n=7). The activity of the biomarkers aspartate aminotransferase (AST) and alanine transaminase (ALT) for liver health showed no significance difference in blood serum for animals given four repeated i.v. injections of XbNPs compared to control animals without treatment (FIG. 8H). Thus, the engineered XbNPs or the presence of the photo-crosslinker Irg do not cause measurable hepatoxicity. This observation is likely due to the intrinsic biodegradability and bioreducibility of the polymers in the XbNP formulation, which allow quick degradation into non-toxic byproducts under aqueous or reducing conditions. Karlsson et al., 2018.

The low toxicity ensures safety of the nanoparticle formulation while also allowing repeated administration for effective therapeutic treatment. If, in future studies in larger animals, Irg becomes a concern, it can be removed using Amicon 10 kDa MWCO filters or similar, prior to administration. Taken together, the ability of the engineered XbNPs to enable systemic delivery to metastatic melanoma tumors could open new avenues for safe and effective siRNA delivery for the unmet need of treatment of metastatic cancers. The differential organ-targeted delivery also could broaden its therapeutic potential for other diseases.

### 3. Summary

Photo-crosslinked bioreducible nanoparticles (XbNP) were designed to address the main issue of colloidal stability of biodegradable cationic polymeric vehicles to broaden their use for systemic siRNA delivery. Bioreducible PBAEs were synthesized to serve both for crosslinking and for payload encapsulation, with disulfide bridges facilitating environmentally triggered intracellular release. Photo-crosslinking provided both improved colloidal nanoparticle stability, which improved payload encapsulation in high serum conditions, and surface charge shielding, which reduced adsorption of anionic serum proteins. XbNPs were observed to demonstrate superior siRNA-mediated knockdown in various glioblastoma cell lines, as well as in melanoma cells compared to non-crosslinked formulations in high-serum conditions. XbNPs are internalized readily by cells, which together with their enhanced stability, explains their great efficacy in high serum. Another key aspect of intracellular trafficking is endosomal escape, for which the presence of both secondary and tertiary amines of XbNPs leads to efficient buffering at low pH, leading to endosomal disruption. In in vivo studies, XbNPs containing labeled siRNA targeted cancer cells and facilitated differential organ-targeted delivery through simple tuning of the polymer/siRNA ratio. In particular, formulations of XbNPs using 900 w/w and 400 w/w formulations accumulated selectively in either the lungs or spleen, respectively, following systemic administration. XbNPs further demonstrated knockdown both when carrying siRNA targeting a reporter gene (Luciferace) and the antiapoptotic gene Bcl-2 after i.v. injections in a metastatic melanoma model, in which tumors colonized the lungs. Taken together, the improved colloidal stability, surface-charge shielding, high transfection efficacy in high-serum conditions, efficient endosomal escape, environmentally-triggered nanoparticle degradation and RNA release, and overall targeted and safe in vivo delivery capacity make XbNPs promising as a robust nanoparticle platform for systemic delivery of RNA therapeutics. The photo-crosslinking strategy also can be applied generally to other cationic nanocarriers for nucleic acid delivery that rely on self-assembly to form nanoparticles for improved stability under physiological conditions.

### 4. Experimental Section

### 4.1 Materials

The chemicals used in the synthesis of the base monomer BR6 were all purchased from Sigma-Aldrich (St. Louis, MO). The other monomers used in the polymer syntheses are as follows: 4-amino-1-butanol (S4; CAS no: 13325-10-05) was purchased from Thermo Fisher Scientific (Carlsbad, CA); 2-(3-aminopropylamino) ethanol (E6; CAS no: 4461-39-6) was purchased from Sigma-Aldrich; and 1-(3-aminopropyl)-4-methylpiperazine (E7; CAS no: 4572-031) was purchased from Alfa Aesar (Ward Hill, MA). The siRNA targeting eGFP with 5′-CAAGCUGACCCUGAAGUUCTT (SEQ ID NO: 1) (sense) and 3′-AACUUCAGGG-UCAGCUUGCC (SEQ ID NO: 2) (antisense) (Ambion Silencer eGFP) and negative control siRNA used as the scrambled RNA (scRNA) with 5′-AGUACUGCUUACGAUACGGTT (SEQ ID NO: 3) (sense) and 3′-CCGUAUCGUAAGCAGUACUTT (SEQ ID NO: 4) (anti-sense) (Ambion Silencer Negative Control #1) were purchased from Thermo Fisher Scientific. The siRNA targeting firefly Luciferase with 5′-AGAAGGAGAUCGUGGACUAUU (SEQ ID NO: 5) (sense) and 3′-UAGUCCACGAUCUCCUUCUUU (SEQ ID NO: 6) (antisense) was purchased from Dharmacon (Lafayette, CO). The siRNA targeting Bcl-2 with 5′-GCAUGCGACCUCUGUUUGATT (SEQ ID NO: 7) (sense) and 3′-UCAAACAGAGGUCGCAUGCTT (SEQ ID NO: 8) (anti-sense) was purchased from Genepharma (Shanghai, China). Cy5-labeled siRNA (SIC005) was purchased from Sigma-Aldrich. Plasmid pCAG-GFPd2 was a gift from Connie Cepko (Addgene plasmid #14760; http://n2t.net/addgene: 14760; RRID:Addgene_14760). Matsuda et al., 2007. PiggyBac transposase expression plasmid (PB200A-1) was purchased from System Biosciences (Palo Alto, CA).

### 4.2 Polymer Synthesis and Photo-Crosslinking

The bioreducible monomer 2,2′-disulfanediylbis(ethane-2,1-diyl)diacrylate (BR6) was synthesized using a method similar to that reported in Kozielski et al., 2013. In brief, 2-hydroxyethyl disulfide (10 mmol) was acrylated in dichloromethane (DCM) with acryloyl chloride as the acrylation reagent (300 mmol) and in the presence of triethylamine (TEA; 300 mmol). Following overnight reaction at room temperature, the TEA HCl precipitate removed by filtration. The product was washed with water and dried with sodium sulfate, and the solvent was removed by rotary evaporation. For the synthesis of the bioreducible PBAE, the diacrylate backbone monomer BR6 and the side chain monomer 4-amino-1-butanol (S4) were dissolved in anhydrous tetrahydrofuran (THF) at a molar ratio of 1.05:1 ratio and a total monomer concentration of 500 mg/mL. The Michael Addition reaction was allowed to proceed for 24 h at 60° C. with stirring. For crosslinker preparation, the resulting acrylate-terminated base polymer R6-4-Ac was precipitated in anhydrous diethyl ether, washed twice with ether, dried under vacuum for 48 h, then dissolved in anhydrous DMSO at 100 mg/mL at −20° C. with desiccant.

For preparation of amine-terminated PBAEs, the acrylate-terminated based polymer was end-capped with either 2-(3-aminopropylamino) ethanol (E6) or 1-(3-aminopropyl)-4-methylpiperazine (E7). The end-capping molecules were dissolved in THF and added to the base polymer (0.5 M final concentration of end-cap and 167 mg/mL of base polymer) and reacted for 1 h at room temperature to form polymers R6-4-6 and R6-4-7. As above, the end-capped polymers were purified by diethyl ether precipitation and two ether washes. The remaining ether was removed by vacuum for 48 h, and the polymers were dissolved in anhydrous DMSO at 100 mg/mL and stored as aliquots at −20° C. with desiccant.

To form the nanoparticles, the polymer and siRNA were diluted separately in 25 mM of sodium acetate buffer (NaAc; pH=5.0) at desired concentrations. In the polymer solution, the acrylate-terminated polymer (R64-Ac) as crosslinker and end-capped polymer (R646 or R647) were mixed together, then mixed with the siRNA solution. Irgacure 2959 (Irg), the radical photoinitiator, was dissolved in NaAc to a final concentration of 1.0 mg/mL. The Irg solution was mixed with the nanoparticles at a 1:1 volume ratio, and the mixture was exposed to UV light (UV lamp F15T8/BL: 15 W and wavelength of 350 nm; EIKO; Shawnee, Canada) for specified times to obtain photo-crosslinked nanoparticles. Irg stock solutions were stored as 100 mg/mL aliquots in DMSO at −20° C. until use.

### 4.3 Characterization of Polymers and Crosslinking: GPC and ¹H NMR

¹H NMR was used to characterize the polymer structures and the degree of crosslinking. The polymers were dissolved in deuterated DMSO (DMSO-d₆) and nanoparticles were first lyophilized and then dissolved in DMSO-do for characterization using a Bruker 500 MHz NMR and analyzed using TopSpin 3.6 software. To determine the degree of crosslinking, the acrylate peaks, which are used to form crosslinks upon exposure to UV in presence of the photoinitiator Irg, were integrated and normalized to protons peaks in the PBAE backbone.

Gel permeation chromatography (GPC) was used to characterize the molecular weight of polymers relative to linear polystyrene standards using a refractive index detector (Waters, Milford, MA). To measure the molecular weight after crosslinking, nanoparticles were formed as described above, and then lyophilized to remove aqueous buffer. Prior to characterization, samples were dissolved in butylated hydroxytoluene (BHT)-stabilized tetrahydrofuran (THF), and filtered through a 0.2 μm polytetrafluoroethylene (PTFE) filter.

### 4.4 Nanoparticle Characterization of Physical Properties and Colloidal Stability

Dynamic light scattering (DLS) using a Zetasizer Pro (Malvern Panalytical) was used to characterize the hydrodynamic diameter of the nanoparticle formulations. The measurements were carried out both in 1×PBS and in 1×PBS with low (10%) or high (50%) serum content. Measurements were carried out in PBS to characterize the influence of polymer concentration and siRNA dose on nanoparticle size and in presence of serum to examine whether crosslinking affected particle size under physiological conditions. To assess the colloidal stability of the particle formulations when incubated in low and high serum conditions, DLS measurements were made for up to 4 h.

Zeta potential measurements were made with the same DLS instrument via electrophoretic mobility to analyze the surface charge of the nanoparticle formulations and to characterized the impact of photo cross-linking, UV exposure time, polymer concentration, and siRNA dose.

Nanoparticle tracking analysis (NTA) and transmission electron microscopy (TEM) were used to further analyze particle size. For the NTA experiments, nanoparticles were diluted in PBS at a 1:150 v/v ratio, then analyzed with a NanoSight NS300 (Malvern, Westborough, MA, USA) to obtain 20-100 particles per frame using the NanoSight NTA 3.2 software. For TEM, nanoparticles were prepared, and 20-μL aliquots were added to carbon-coated copper TEM grids (Electron Microscopy Sciences, Hatfield, PA, USA), then grids were washed three times for 10 seconds each with MilliQ water, and thereafter dried at room temperature for 10 min before they were imaged.

Lastly, a gel electrophoresis assay was performed to investigate the stability and bioreducible nature of the crosslinked particles. The crosslinked and non-crosslinked nanoparticles using 1200, 900, 300, and 200 were compared after incubation in either 50% serum for 0, 2, and 4 h or a reducing environment (10 mM of glutathione, GSH) for 0.25, 0.5, and 1 h. Serum alone and siRNA alone were used as controls. Samples and controls containing 167 nM siRNA except in the serum-only control were loaded in an 1% agarose (UltraPure Agarose, Invitrogen, Carlsbad, CA) gel with 0.001 mg/mL ethidium bromide, and the gel was run for 20 min at 100 V and imaged with UV light exposure.

### 4.5 Protein Adsorption: BCA and SDS-PAGE

For protein adsorption evaluation, nanoparticles were incubated with 100% serum or with PBS as a control in 1.5 mL-tubes (LoBind, Eppendorf) for 1 h at 37° C. The mixture was centrifuged at 18,000 g for 1 h at 4° C., and the pellet was washed and then resuspended in PBS. The protein concentration was then measured using the BCA assay following the manufacturer's instructions. For analysis of individual proteins, SDS-PAGE analysis was carried out using 4-15% Mini-PROTEAN TGX Precast Protein Gels (Bio-Rad) with 1×Tris/Glycine/SDS (Bio-Rad) as the running buffer. Gel electrophoresis was run at 150 V for 45 min in a Mini-PROTEAN Tetra cell (Bio-Rad).

### 4.6 Cell Culture and Cell Line Preparation

GBM319 patient-derived glioblastoma cells, GL261 murine glioma cells, and B16-F10 murine melanoma cells were cultured in Dulbecco's modified Eagle's medium (DMEM; Thermo Fisher Scientific) supplemented with 10% fetal bovine serum (FBS) and 1% penicillin/streptomycin. GBM1A patient-derived glioblastoma cells were cultured as neurospheres in DMEM-F12 medium (Life Technologies) with 2% (v/v) B-27 Serum-Free Supplement (Invitrogen), 1% (v/v) Antibiotic-Antimycotic, 20 ng/mL epidermal growth factor (PeproTech, Rocky Hill, NJ, USA), and 10 ng/mL basic fibroblast growth factor (PeproTech). For generation of reporter cell lines for optical readout for siRNA transfection experiments, a PiggyBac transposon/transposase system was used to generate cell lines constitutively expressing a destabilized form of GFP (GFPd2), Matsuda et al., 2007, as described previously. Rui et al., 2019a.

The PiggyBac transposon plasmid PB-CAG-GFPd2 was constructed in a laboratory and is available on Addgene (Addgene plasmid #115665; RRID: Addgene_115665). Rui et al., 2019a. The PiggyBac transposon plasmid used to induce cells to express a firefly luciferase-tdTomato fusion protein (PB-fLuc-tdT, Addgene plasmid #120870; http://n2t.net/addgene: 120870; RRID: Addgene_120870) was prepared from PBGFPd2 and pcDNA3.1 (+)/Luc2-tdT using restriction enzyme cloning. pcDNA3.1 (+)/Luc2=tdT was a gift from Christopher Contag (Addgene plasmid #32904; RRID: Addgene_32904). Patel et al., 2010. PiggyBac transposase expression plasmid (PB200A-1) was purchased from System Biosciences (Palo Alto, CA). Cell lines were stably induced to express PiggyBac transposon expression cassettes as previously described and sorted to stably expressing population of cells using a Sony SH800 cytometer as previously described. Rui et al., 2019a.

4.7 siRNA Delivery In Vitro: Transfection and Viability

Cells were seeded into 96-well tissue culture plates at a density of either 5,000 (B16F10), 15,000 (GBM319 and GL261), or 20,000 (GBMIA) cells/well in 100 μL complete medium and allowed to adhere overnight. GBM1A neurospheres were first dissociated into single cells and plated into wells coated with 5 μg/mL laminin (Sigma) for 3 h at 37° C., and cells were allowed to adhere for 48 h. Nanoparticles were formed in 25-mM NaAc. For the crosslinked formulations, photoinitiator was added and UV exposure applied as described above. Each nanoparticle condition was formulated with either siRNA targeting GFP or a scrambled control siRNA (scRNA). Prior to the addition of nanoparticles, the cell culture medium was replaced with 100 μL of complete media with specified serum content (10-100%). Nanoparticles were added to each well at a 1:5 ratio of nanoparticles to medium, with a final RNA concentration of 20-120 nM per well, and allowed to incubate with cells at 37° C. for 2 h, after which the mixture of particles and media was replaced with fresh complete medium. Flow cytometry was performed using a BD Accuri C6 flow cytometer (BD Biosciences) with HyperCyt autosampler to assess knockdown of GFP expression after 2 (B16F10), 4 (GL261), 5 (GBMIA), or 7 (GBM319) days. Knockdown efficacy was quantified by normalizing the geometric mean fluorescence of cells treated with siRNA to that of cells transfected with corresponding formulation containing scRNA in FlowJo (n=4).

The MTS CellTiter 96 Aqueous One (Promega, Madison, WI) cell proliferation assay was performed 24 h post-transfection according to manufacturer's instructions as a measure of cell viability. The metabolic activity of treated cells was normalized to that of untreated cells (n=4).

4.8 Cellular Uptake and Endosomal Escape: Flow Cytometry, Confocal Microscopy, and Gal8 assay

In the experiments for cellular uptake, formulations were prepared with 20% Cy5-labeled siRNA and 80% unlabeled siRNA. The nanoparticles were added to cells in media with specified serum content (50% or 100%) and allowed to incubate for 2 h, after which cells were washed with PBS and detached via trypsinization. Cells were further washed with heparin (50 μg/mL in PBS) to remove surface-bound nanoparticles and were thereafter resuspended in FACS buffer (2% FBS in PBS) for flow cytometry analysis to quantify nanoparticle uptake.

Confocal microscopy was used to visualize nanoparticle uptake and endosomal escape. GBM319 cells were plated on Nunc Lab-Tek 8-chambered borosilicate cover-glass well plates (155411; Thermo Fisher Scientific) at 30,000 cells/well one day prior to transfection in 250 μL media with specified serum content (50% or 100%). The nanoparticles were prepared as described above with 20% Cy5-labeled siRNA and 80% unlabeled siRNA, and 50 μL was administered to each well and incubated with cells for 2 h. After particles and media were replaced with fresh complete media, and prior to imaging, cells were stained for 30 min with Hoechst 33342 (Thermo Fisher Scientific) nuclear stain at a 1:5000 dilution and Cell Navigator Lysosome Staining dye (AAT Bioquest, Sunnyvale, CA) at a 1:2000 dilution. Cells were washed twice and incubated in phenol-red free media, and live-cell imaging was performed at 37° C. in 5% CO₂. Images were acquired using a Zeiss LSM 780 microscope with Zen Blue software and a 63× oil immersion lens. Specific laser channels used were 405 nm diode, 488 nm argon, 561 nm solid-state, and 639 nm diode lasers. Laser intensity and detector gain settings were maintained across all image acquisitions.

The Gal8-GFP recruitment assay was performed to assess endosomal disruption/endosomal escape of nanoparticles based on a method recently reported by Kilchrist et al., 2019. Briefly, B16F10 cells were engineered to constitutively express a Gal8-GFP fusion protein using the PiggyBac transposon plasmid PB-GFP-Gal8 constructed in our lab (Addgene plasmid #127191; RRID: Addgene_127191). Rui et al., 2019b.

Nanoparticles encapsulating 20% Cy5-labeled siRNA and 80% unlabeled siRNA were incubated with cells for 2 h in media with 50% serum, after which media were replaced with fresh complete media and stained with Hoechst 33342 nuclear stain (1:5000 dilution). Gal8-GFP recruitment was analyzed using a Cellomics ArrayScan VTI with live-cell imaging module; cell count was generated using an algorithm to extrapolate the area surrounding Hoechst-stained cell nuclei, endosomal disruption was reported as the average number of punctate Gal8-GFP spots per cell, and cellular uptake reported as the average number of Cy5 spots per cell.

### 4.9 Animals

For the in vivo studies, 6- to 8-week-old female C₅₇BL/6J mice (Jackson Laboratory, Bar Harbor, ME, USA) were housed in standard facilities and were supplied with ad libitum access to food and water. All animal studies were performed in strict accordance with the NIH guidelines for the care and use of laboratory animals (NIH Publication No. 85-23 Rev. 1985). The laboratory, investigators, and procedures were approved with animal protocol MO18M388 by the Animal Care and Use Committee (ACUC) of the Johns Hopkins University.

### 4.10 In Vivo Lung Metastasis Model, Nanoparticle Biodistribution, and Cellular Uptake.

To establish metastasis-like lesions in the lungs, 100,000 B16-F10 cells suspended in 100 μL of PBS were injected i.v. into mice by the lateral tail vein. To allow an optical readout of the tumor formation, B16F10-Luc=tdTomato cells were used, and tumor growth was monitored via IVIS imaging. In the biodistribution study, 7 days after tumor inoculation, nanoparticles encapsulating siRNA labeled with IR fluorescent dye (IRDye800CW; Integrated DNA Technologies, Coralville, IA, USA) were injected i.v. (n=3-5). A control group (n=3) was not injected with nanoparticles to account for the contribution of autofluorescence of the organs. The animals were euthanized 18 h post-administration, and the organs were collected. IVIS (PerkinElmer, Waltham, MA, USA) imaging was used to analyze the biodistribution of the fluorescent nanoparticles, and the images were analyzed in Living Image software (PerkinElmer).

In the study of cellular uptake in the lungs, tumors were established as described above using B16-F10 GFPd2+ cells. After 14 days, nanoparticles encapsulating Cy5-labeled siRNA were injected i.v. (n=4) to allow analysis of cellular uptake, and untreated animals were included as controls (n=2). Mice were euthanized 18 h post-administration, and lungs were collected, minced, and dissociated using the Lung Dissociation kit (Miltenyi Biotec) according to the manufacturer's instructions. Red blood cells were lysed in ACK buffer, and the remaining cells were incubated for 30 min at 4° C. with antibodies against epithelial (CD326-APC/Cy7), endothelial (CD31-BV421), and immune (CD45-BV421) cell markers (all antibodies from BioLegend, San Diego, CA, USA), see Table 1 for details. The cells were then analyzed using a CytoFlex flow cytometer (Beckman Coulter).

TABLE 1
Antibodies used to assess cellular uptake of nanoparticles in the lungs.

| | | | | | Catalog | Lot |
|---|---|---|---|---|---|---|
| Antigen | Color | Supplier | Clone | Dilution | No. | No. |
| CD45 | Brilliant | Biolegend | 30-F11 | 1:100 | 103134 | B287242 |
| | Violet 421 | | | | | |
| CD31/PECAM | Alexa | Biolegend | 390 | 1:100 | 102443 | B303280 |
| | Fluor 700 | | | | | |
| CD326/EpCAM | APC/Cy7 | Biolegend | G8.8 | 1:80 | 118218 | B266989 |

### 4.11 Efficacy and Safety for Systemically Administrated Nanoparticles

To evaluate siRNA-mediated knockdown following systemic nanoparticle administration, the bioluminescence signal from the B16F10 tumors in the lungs was monitored by IVIS. Prior to imaging, 3.75 mg D-luciferin (Cayman Chemical Company) in 150 μL volume was injected intraperitoneally (i.p.) in each mouse. Image analysis was carried out using Living Image software to quantify the total bioluminescence of the colonized tumors. The bioluminescence signal at the specific time-points were normalized to the initial signal prior to treatment (day 7). The nanoparticles were loaded with either siRNA targeting firefly luciferase (siLuc), siRNA targeting Bcl-2 (siBcl-2), or negative control siRNA (scRNA), and bioluminescence was used to determine whether successful delivery was achieved.

Any potential hepatotoxicity of the systemically delivered photo-crosslinked nanoparticles was examined. Animals received four repeated i.v. injections of nanoparticles and untreated animals were used as controls. Blood was collected from each animal after 8 days after initiation of treatment, and the serum was collected by centrifugation at 1,500 rcf for 15 min at 4° C. The serum in treated and untreated animals was analyzed for aspartate aminotransferase (AST) activity and alanine transaminase (ALT) (Sigma-Aldrich), key biomarkers for liver health. The AST and ALT activity assays were performed according to manufacturer's instructions.

### 4.12 Statistical Analyses

All results are presented as mean±standard error of the mean (SEM). All statistical analyses were performed using Prism software (Graphpad Prism, San Diego, CA, USA), and p<0.05 was considered statistically significant. For the analysis of the physical nanoparticle properties one-way ANOVA followed by Tukey's post hoc test was used to compare nanoparticle size and surface charge of the different formulations. For the analysis of the total protein adsorption, two-way ANOVA followed by Sidak's multiple comparisons was used. For the analyses of in vitro transfection and viability, one-way ANOVA followed by Tukey's post hoc test was used to compare the influence of UV exposure time, PBAE concentration, siRNA dose, and Irg concentration. Additionally, when comparing the transfection, cell uptake, and encapsulation efficiency of crosslinked and non-crosslinked formulations, and in the pre-incubation experiment two-way ANOVA followed by Sidak's multiple comparisons was used. For the encapsulation efficiency assessed by the RiboGreen assay, Holm-Sidak corrected multiple t tests was used. For the analysis of nanoparticle uptake in the Gal8-GFP assay, a two-tailed Student's t-test was used. For the analysis of the in vivo biodistribution, a two-way ANOVA followed by Sidak's multiple comparisons was used to compare the fluorescent intensity of harvested organs. For the analysis of nanoparticle uptake in vivo, one-way ANOVA followed by Tukey's post hoc test was used to compare uptake of different cell-types in the lungs. For the analysis of siRNA-mediated knockdown in metastatic B16F10-Luc⁺ tumors, a two-way ANOVA followed by Dunnett's multiple comparison was used to examine in vivo delivery efficacy of XbNPs. For the analyses of AST and ALT activities and tdTomato expression 15 days after tumor inoculation, two-tailed Student's t-test was used.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
H. Yin, R. L. Kanasty, A. A. Eltoukhy, A. J. Vegas, J. R. Dorkin, D. G. Anderson, Nat. Rev. Genet. 2014, 15, 541.
K. A. Whitehead, R. Langer, D. G. Anderson, Nat. Rev. Drug Discov. 2009, 8, 129.
N. Bessis, F. J. GarciaCozar, M. C. Boissier, Gene Ther. 2004, 11, S10.
C. E. Thomas, A. Ehrhardt, M. A. Kay, Nat. Rev. Genet. 2003, 4, 346.
D. W. Pack, A. S. Hoffman, S. Pun, P. S. Stayton, Nat. Rev. Drug Discov. 2005, 4, 581.
M. A. Mintzer, E. E. Simanek, Chem. Rev. 2009, 109, 259.
D. Adams, A. Gonzalez-Duarte, W. D. O'Riordan, C. C. Yang, M. Ueda, A. V Kristen, I. Tournev, H. H. Schmidt, T. Coelho, J. L. Berk, K. P. Lin, G. Vita, S. Attarian, V. Plante-Bordeneuve, M. M. Mezei, J. M. Campistol, J. Buades, T. H. Brannagan, B. J. Kim, J. Oh, Y. Parman, Y. Sekijima, P. N. Hawkins, S. D. Solomon, M. Polydefkis, P. J. Dyck, P. J. Gandhi, S. Goyal, J. Chen, A. L. Strahs, S. V Nochur, M. T. Sweetser, P. P. Garg, A. K. Vaishnaw, J. A. Gollob, O. B. Suhr, N. Engl. J. Med. 2018, 379, 11.
A. Akinc, M. A. Maier, M. Manoharan, K. Fitzgerald, M. Jayaraman, S. Barros, S. Ansell, X. Y. Du, M. J. Hope, T. D. Madden, B. L. Mui, S. C. Semple, Y. K. Tam, M. Ciufolini, D. Witzigmann, J. A. Kulkarni, R. van der Meel, P. R. Cullis, Nat. Nanotechnol. 2019, 14, 1084.
M. Longmire, P. L. Choyke, H. Kobayashi, Nanomedicine 2008, 3, 703.
K. L. Kozielski, S. Y. Tzeng, J. J. Green, Chem. Commun. 2013, 49, 5319.
K. L. Kozielski, S. Y. Tzeng, B. A. Hurtado De Mendoza, J. J. Green, ACS Nano 2014, 8, 3232.
J. Karlsson, Y. Rui, K. L. Kozielski, A. L. Placone, O. Choi, S. Y. Tzeng, J. Kim, J. J. Keyes, M. I. Bogorad, K. Gabrielson, H. Guerrero-Cazares, A. Quinones-Hinojosa, P. C. Searson, J. J. Green, Nanoscale 2019, 11, 20045.
J. Karlsson, K. R. Rhodes, J. J. Green, S. Y. Tzeng, Expert Opin. Drug Deliv. 2020, 17425247.2020.1796628.
D. Routkevitch, D. Sudhakar, M. Conge, M. Varanasi, S. Y. Tzeng, D. R. Wilson, J. J. Green, ACS Biomater. Sci. Eng. 2020, 6, 3411.
D. R. Wilson, Y. Rui, K. Siddiq, D. Routkevitch, J. J. Green, Mol. Pharm. 2019, 16, 655.
M. Morille, C. Passirani, A. Vonarbourg, A. Clavreul, J. P. Benoit, Biomaterials 2008, 29, 3477.
F. Alexis, E. Pridgen, L. K. Molnar, O. C. Farokhzad, Mol. Pharm. 2008, 5, 505.
A. Sizovs, L. Xue, Z. P. Tolstyka, N. P. Ingle, Y. Wu, M. Cortez, T. M. Reineke, J. Am. Chem. Soc. 2013, 135, 15417.
L. Novo, E. V. B. van Gaal, E. Mastrobattista, C. F. van Nostrum, W. E. Hennink, J. Control. Release 2013, 169, 246.
J. Karlsson, H. J. Vaughan, J. J. Green, Annu. Rev. Chem. Biomol. Eng. 2018, 9, 105.
H. Hatakeyama, H. Akita, E. Ito, Y. Hayashi, M. Oishi, Y. Nagasaki, R. Danev, K. Nagayama, N. Kaji, H. Kikuchi, Y. Baba, H. Harashima, Biomaterials 2011, 32, 4306.
R. K. O′reilly, C. J. Hawker, K. L. Wooley, Chem. Soc. Rev. 2006, 35, 1068.
N. A. Peppas, J. Z. Hilt, A. Khademhosseini, R. Langer, Adv. Mater. 2006, 18, 1345.
W. E. Hennink, C. F. van Nostrum, Adv. Drug Deliv. Rev. 2012, 64, 223.
S. Y. Tzeng, E. B. Lavik, Soft Matter 2010, 6, 2208.
O. Z. Fisher, N. A. Peppas, Macromolecules 2009, 42, 3391.
O. Z. Fisher, T. Kim, S. R. Dietz, N. A. Peppas, Pharm. Res. 2009, 26, 51.
D. C. Forbes, N. A. Peppas, ACS Nano 2014, 8, 2908.
K. M. Luly, J. Choi, Y. Rui, J. J. Green, E. M. Jackson, Nanomedicine (Lond). 2020.
G. M. Whitesides, J. P. Mathias, C. T. Seto, Science (80-.). 1991, 254, 1312.
S. Zhang, Nat. Biotechnol. 2003, 21, 1171.
D. Anderson, D. Lynn, R. Langer, Angew. Chemie 2003, 115, 3261.
J. J. Green, R. Langer, D. G. Anderson, Acc. Chem. Res. 2008, 41, 749.
J. Sunshine, J. J. Green, K. P. Mahon, F. Yang, A. A. Eltoukhy, D. N. Nguyen, R. Langer, D. G. Anderson, Adv. Mater. 2009, 21, 4947.
A. Mangraviti, S. Y. Tzeng, K. L. Kozielski, Y. Wang, Y. Jin, D. Gullotti, M. Pedone, N. Buaron, A. Liu, D. R. Wilson, S. K. Hansen, F. J. Rodriguez, G. D. Gao, F. Dimeco, H. Brem, A. Olivi, B. Tyler, J. J. Green, ACS Nano 2015, 9, 1236.
H. Lopez-Bertoni, K. L. Kozielski, Y. Rui, B. Lal, H. Vaughan, D. R. Wilson, N. Mihelson, C. G. Eberhart, J. Laterra, J. J. Green, Nano Lett. 2018, 18, 4086.
Y. Wang, M. Ye, R. Xie, S. Gong, Bioconjug. Chem. 2019, 30, 325.
M. Goldberg, R. Langer, X. Jia, J. Biomater. Sci. Polym. Ed. 2007, 18, 241.
T. L. Moore, L. Rodriguez-Lorenzo, V. Hirsch, S. Balog, D. Urban, C. Jud, B. Rothen-Rutishauser, M. Lattuada, A. Petri-Fink, Chem. Soc. Rev 2015, 44, 6287.
E. Blanco, H. Shen, M. Ferrari, Nat. Biotechnol. 2015, 33, 941.
J. S. Suk, Q. G. Xu, N. Kim, J. Hanes, L. M. Ensign, Adv. Drug Deliv. Rev. 2016, 99, 28.
S. D. Li, L. Huang, Stealth nanoparticles: High density but sheddable PEG is a key for tumor targeting. J. Control. Release 2010, 145, 178-181.
T. Cedervall, I. Lynch, S. Lindman, T. Berggård, E. Thulin, H. Nilsson, K. A. Dawson, S. Linse, Proc. Natl. Acad. Sci. U.S.A 2007, 104, 2050.
D. Docter, D. Westmeier, M. Markiewicz, S. Stolte, S. K. Knauer, R. H. Stauber, Chem. Soc. Rev. 2015, 44, 6094.
P. C. Ke, S. Lin, W. J. Parak, T. P. Davis, F. Caruso, ACS Nano 2017, 11, 11773.
Y. Zhang, J. L. Y. Wu, J. Lazarovits, W. C. W. Chan, J. Am. Chem. Soc. 2020, 142, 8827.
H. Hyun, J. Park, K. Willis, J. E. Park, L. T. Lyle, W. Lee, Y. Yeo, Biomaterials 2018, 180, 206.
H. Cao, L. Zou, B. He, L. Zeng, Y. Huang, H. Yu, P. Zhang, Q. Yin, Z. Zhang, Y. Li, Adv. Funct. Mater. 2017, 27, 1605679.
D. S. Manickam, J. Li, D. A. Putt, Q. H. Zhou, C. Wu, L. H. Lash, D. Oupický, J. Control. Release 2010, 141, 77.
F. Meng, W. E. Hennink, Z. Zhong, Biomaterials 2009, 30, 2180.
S. Mura, J. Nicolas, P. Couvreur, Nat. Mater. 2013, 12, 991.
Y. Kakizawa, A. Harada, K. Kataoka, J. Am. Chem. Soc. 1999, 121, 11247.
S. Son, R. Namgung, J. Kim, K. Singha, W. J. Kim, Acc. Chem. Res. 2012, 45, 1100.
X. Liu, J. Xiang, D. Zhu, L. Jiang, Z. Zhou, J. Tang, X. Liu, Y. Huang, Y. Shen, Adv. Mater. 2016, 28, 1743.
A. Sukhanova, S. Bozrova, P. Sokolov, M. Berestovoy, A. Karaulov, I. Nabiev, Nanoscale Res. Lett. 2018, 13, 44.
A. Soeda, A. Hara, T. Kunisada, S. I. Yoshimura, T. Iwama, D. M. Park, Sci. Rep. 2015, 5, 7979.
J. Tilghman, H. Wu, Y. Sang, X. Shi, H. Guerrero-Cazares, A. Quinones-Hinojosa, C. G. Eberhart, J. Laterra, M. Ying, Cancer Res. 2014, 74, 3168.
S. Bao, Q. Wu, R. E. Mclendon, Y. Hao, Q. Shi, A. B. Hjelmeland, M. W. Dewhirst, D. D. Bigner, J. N. Rich, Nature 2006, 444, 756.
D. M. Park, J. N. Rich, Mol. Cells 2009, 28, 7.
S. A. Smith, L. I. Selby, A. P. R. Johnston, G. K. Such, Bioconjug. Chem. 2019, 30, 263.
L. M. P. Vermeulen, S. C. De Smedt, K. Remaut, K. Braeckmans, The proton sponge hypothesis: Fable or fact? Eur. J. Pharm. Biopharm. 2018, 129, 184-190.
K. V Kilchrist, S. C. Dimobi, M. A. Jackson, B. C. Evans, T. A. Werfel, E. A. Dailing, S. K. Bedingfield, I. B. Kelly, C. L. Duvall, ACS Nano 2019, 13, 1136.
Q. Cheng, T. Wei, L. Farbiak, L. T. Johnson, S. A. Dilliard, D. J. Siegwart, Nat. Nanotechnol. 2020, 15, 313.
B. Zbytek, J. A. Carlson, J. Granese, J. Ross, M. C. Mihm, A. Slominski, Expert Rev Dermatol 2008, 3, 569.
C. G. Zamboni, K. L. Kozielski, H. J. Vaughan, M. M. Nakata, J. Kim, L. J. Higgins, M. G. Pomper, J. J. Green, J. Control. Release 2017, 263, 18.
S. Y. Tzeng, L. J. Higgins, M. G. Pomper, J. J. Green, J. Biomed. Mater. Res.-Part A 2013, 101 A, 1837.
S. Y. Tzeng, H. Guerrero-Cázares, E. E. Martinez, J. C. Sunshine, A. QuiñonesHinojosa Alfredo, J. J. Green, Biomaterials 2011, 32, 5402.
A. M. Merlot, D. S. Kalinowski, D. R. Richardson, Front. Physiol. 2014, 5 Aug., 299.
M. S. Dennis, H. Jin, D. Dugger, R. Yang, L. McFarland, A. Ogasawara, S. Williams, M. J. Cole, S. Ross, R. Schwall, Cancer Res. 2007, 67, 254.
C. F. A. Warren, M. W. Wong-Brown, N. A. Bowden, Cell Death Dis. 2019, 10, 1.
Z. Zhou, H. Li, K. Wang, Q. Guo, C. Li, H. Jiang, Y. Hu, D. Oupicky, M. Sun, ACS Appl. Mater. Interfaces 2017, 9, 14576.
A. Y. Bedikian, M. Millward, H. Pehamberger, R. Conry, M. Gore, U. Trefzer, A. C. Pavlick, R. DeConti, E. M. Hersh, P. Hersey, J. M. Kirkwood, F. G. Haluska, J. Clin. Oncol. 2006, 24, 4738.
T. Matsuda, C. L. Cepko, Proc. Natl. Acad. Sci. U.S.A 2007, 104, 1027.
Y. Rui, D. R. Wilson, K. Sanders, J. J. Green, ACS Appl. Mater. Interfaces 2019a, 11, 10472.
M. R. Patel, Y. F. Chang, I. Y. Chen, M. H. Bachmann, X. Yan, C. H. Contag, S. S. Gambhir, Cancer Res. 2010, 70, 10141.
Y. Rui, D. R. Wilson, J. Choi, M. Varanasi, K. Sanders, J. Karlsson, M. Lim, J. J. Green, Sci. Adv. 2019b, 5, eaay3255.
C. G. Williams, A. N. Malik, T. K. Kim, P. N. Manson, J. H. Elisseeff, Biomaterials 2005, 26, 1211.
T. Lin, P. Zhao, Y. Jiang, Y. Tang, H. Jin, Z. Pan, H. He, V. C. Yang, Y. Huang, ACS Nano 2016, 10, 9999.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A nanoparticle comprising a bioreducible cationic polymer, a crosslinking polymer and one or more nucleic acids, wherein the bioreducible cationic polymer and crosslinking polymer are crosslinked and the one or more nucleic acids are encapsulated within the nanoparticle.

2. The nanoparticle of claim 1, wherein the bioreducible cationic polymer comprises a bioreducible poly(beta-amino ester).

3. The nanoparticle of claim 2, wherein the bioreducible poly(beta-amino ester) comprises at least one degradable ester bond and at least one disulfide bond.

4. The nanoparticle of claim 2 or claim 3, wherein the bioreducible poly(beta-amino ester) comprises an amine-terminated bioreducible poly(beta-amino ester).

5. The nanoparticle of claim 4, wherein the amine-terminated bioreducible poly(beta-amino ester) comprises a monomer having the following structure:

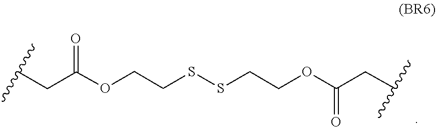 [SMILES: *CC(=O)OCCSSCCOC(=O)C*]

6. The nanoparticle of claim 5, wherein the amine-terminated bioreducible poly(beta-amino ester) comprises a compound having the following structure: 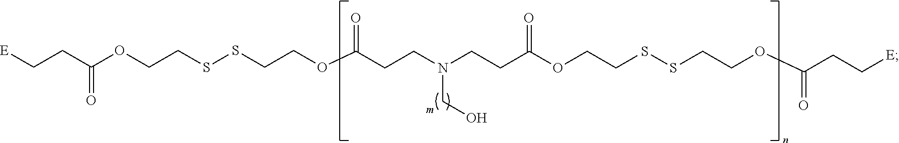
wherein:
n is an integer from 1 to 10,000;
m is an integer selected from the group consisting of 2, 3, 4, 5, 6, 7, 8, and 9; and
E is an amine-containing endcapping group.

7. The nanoparticle of claim 6, wherein the amine-containing endcapping group is selected from the group consisting of:

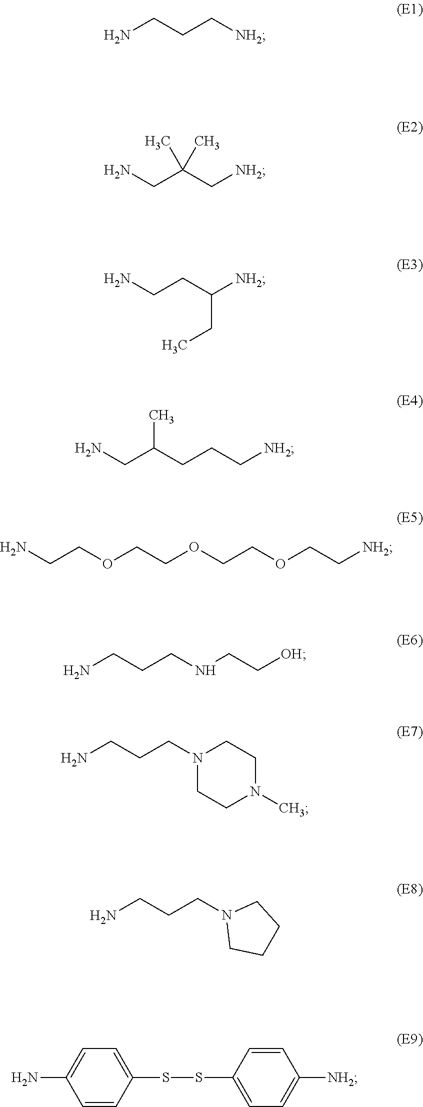

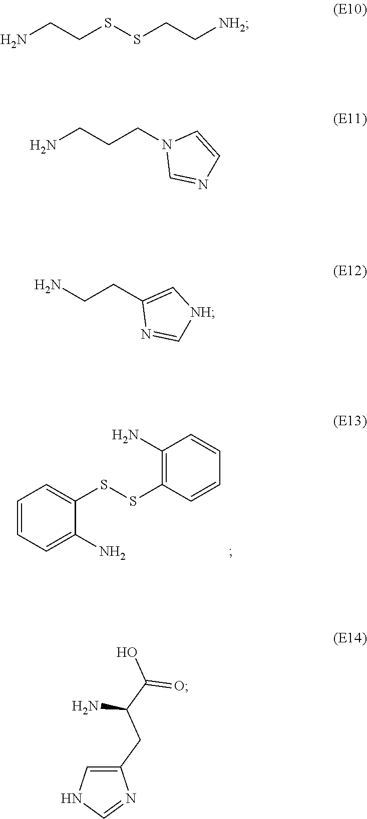 [SMILES: NCCSSCCN ; NCCCn1ccnc1 ; NCCc1c[nH]cn1 ; Nc1ccccc1SSc1ccccc1N ; N[C@H](Cc1c[nH]cn1)C(=O)O]

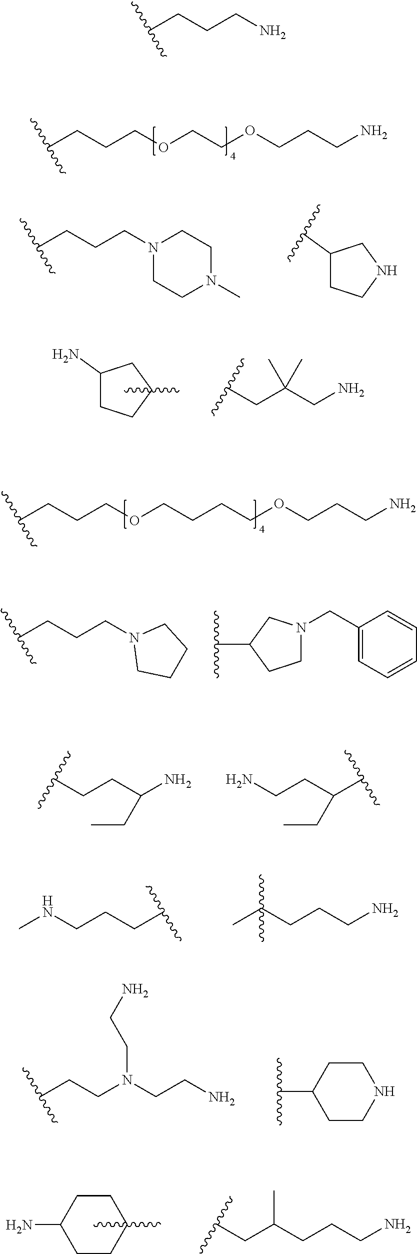

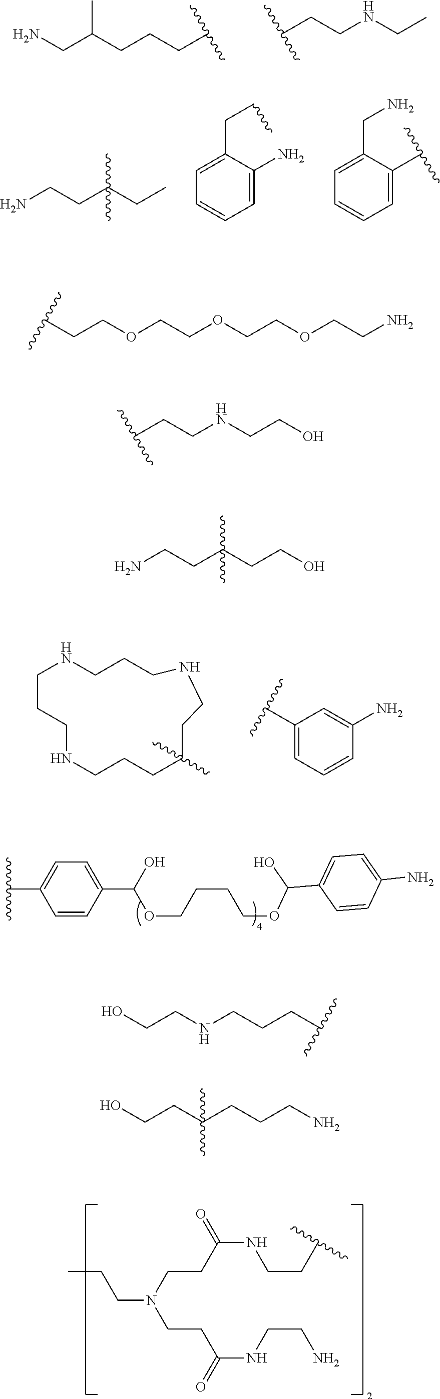

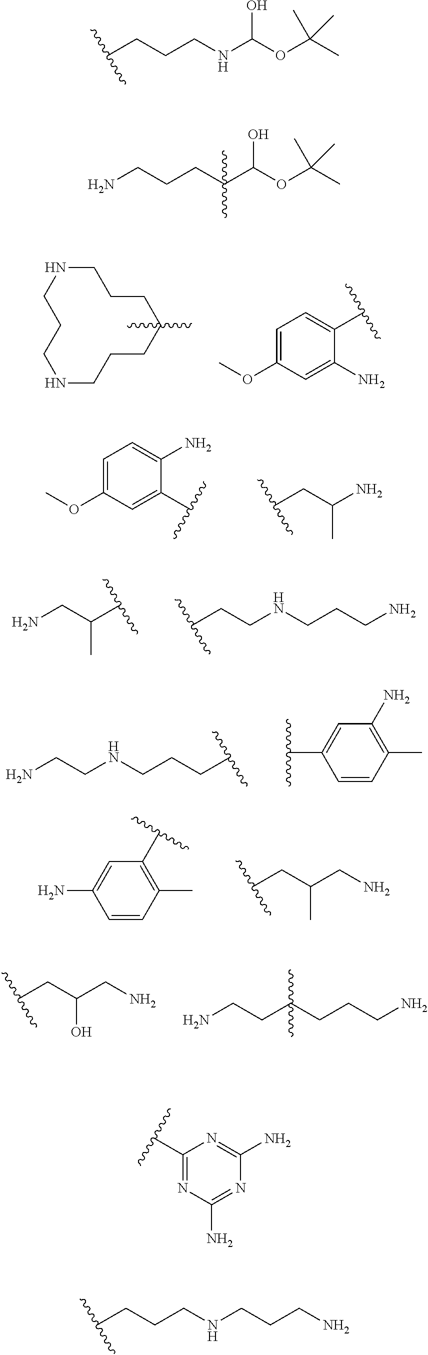

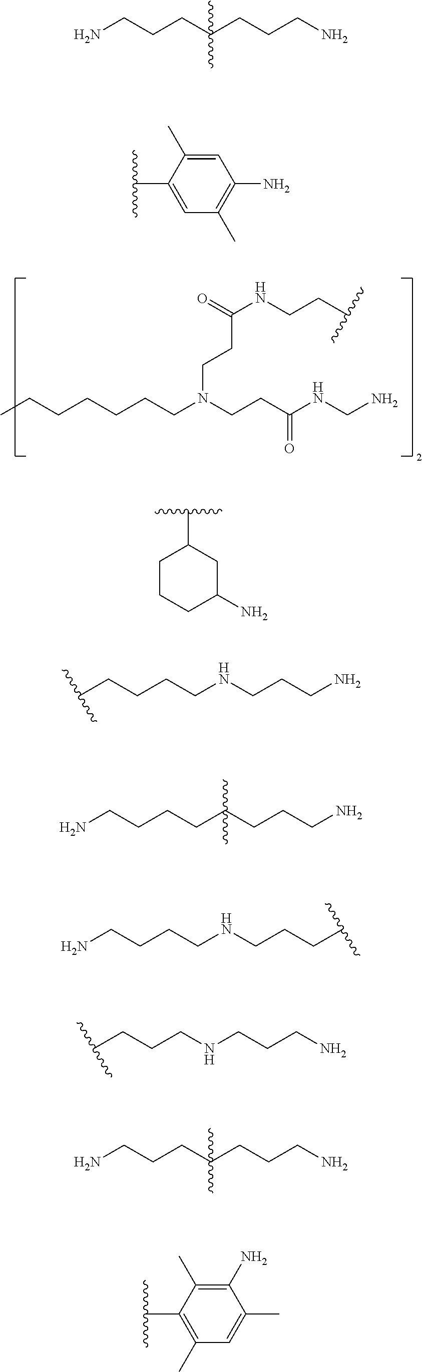

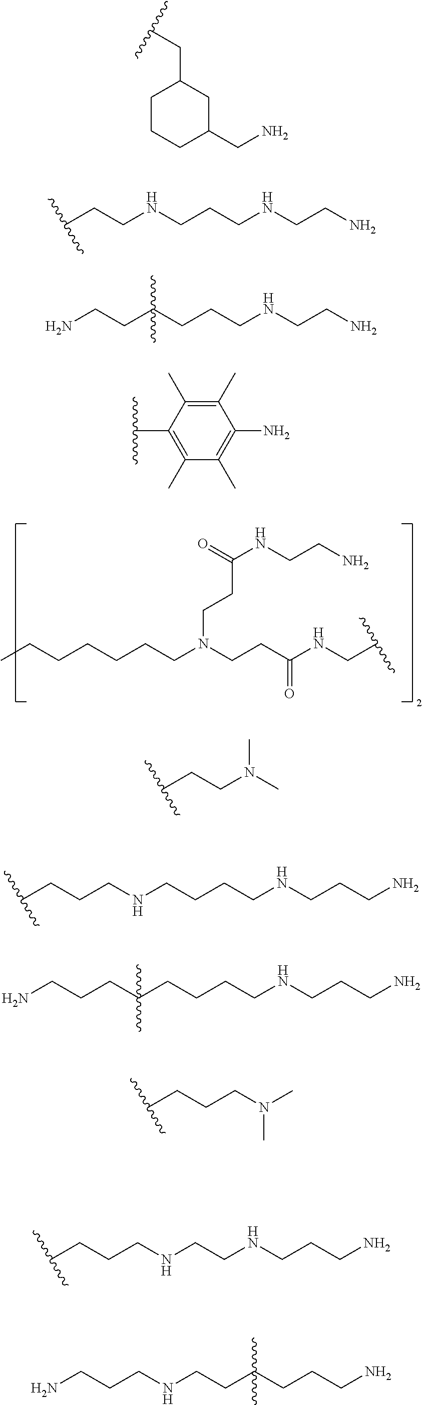

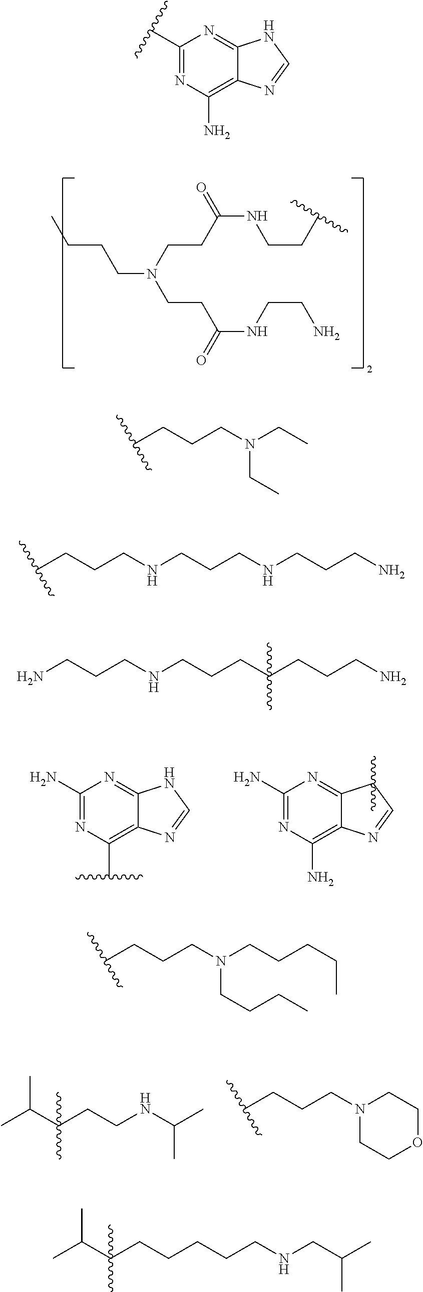

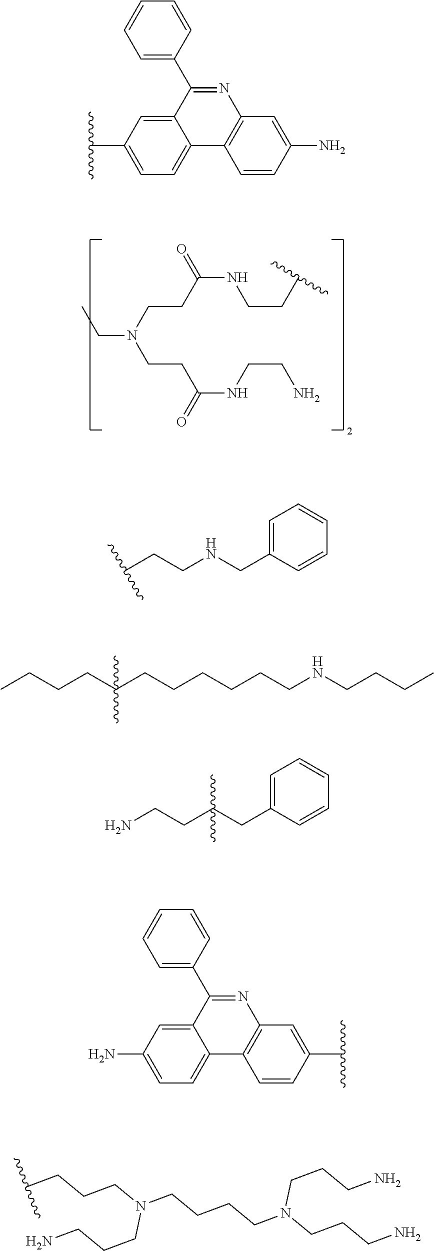

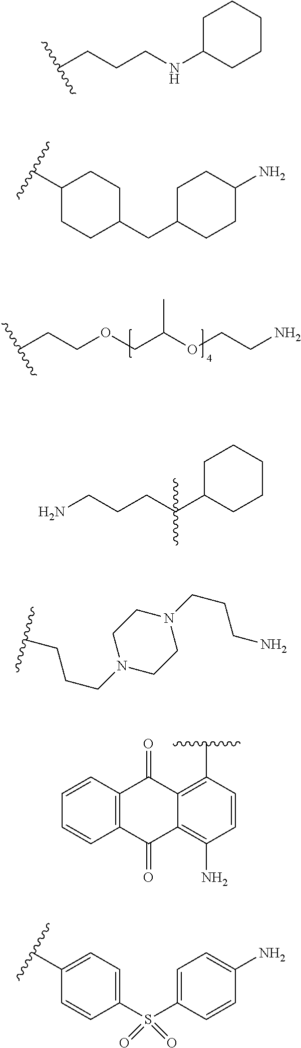

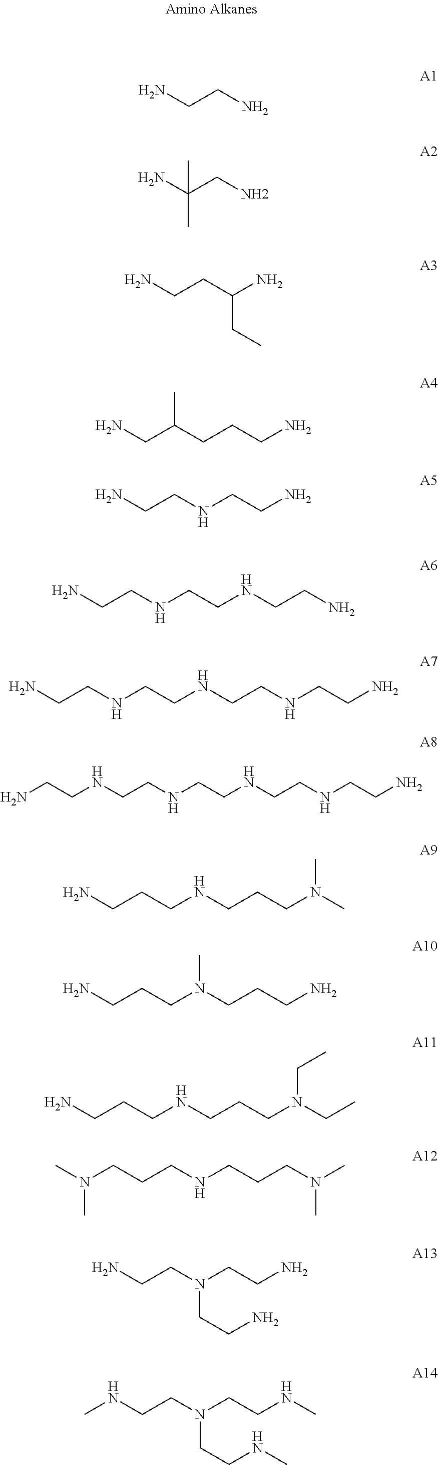

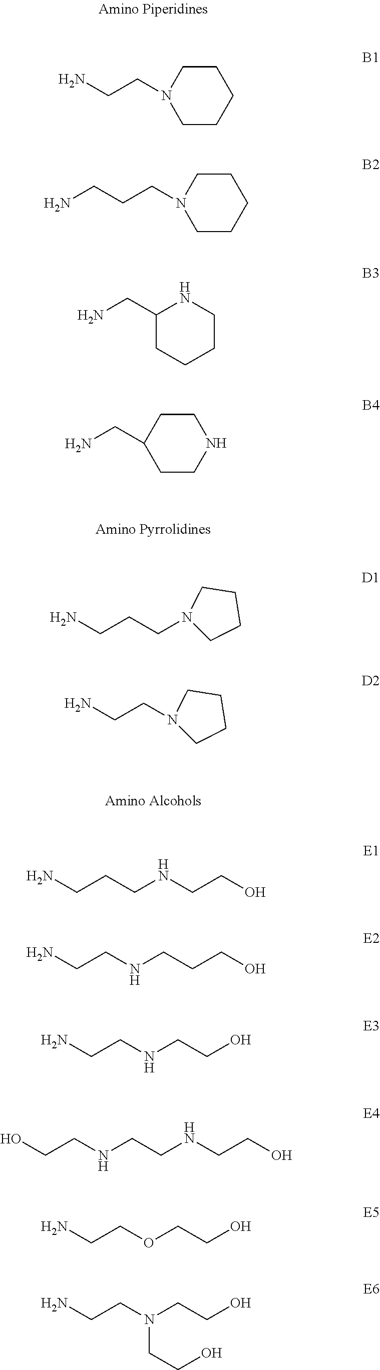

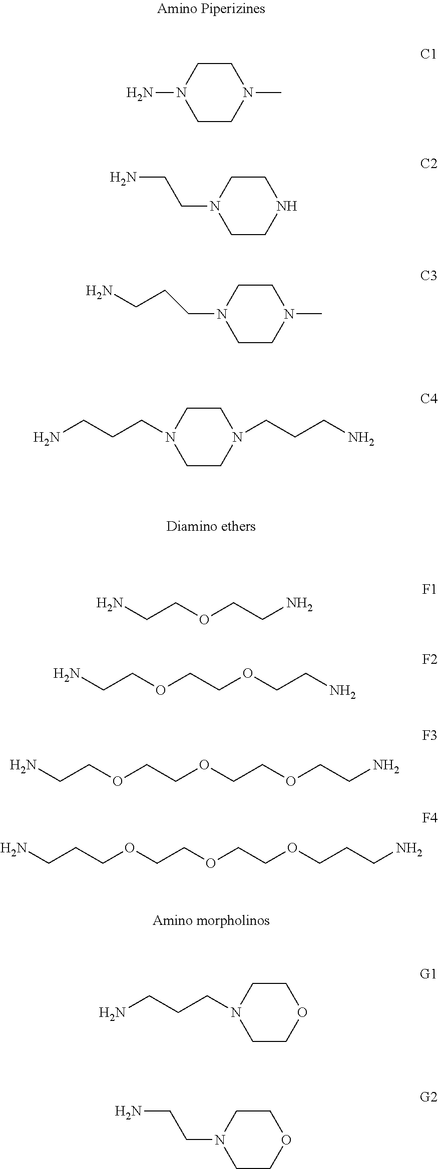

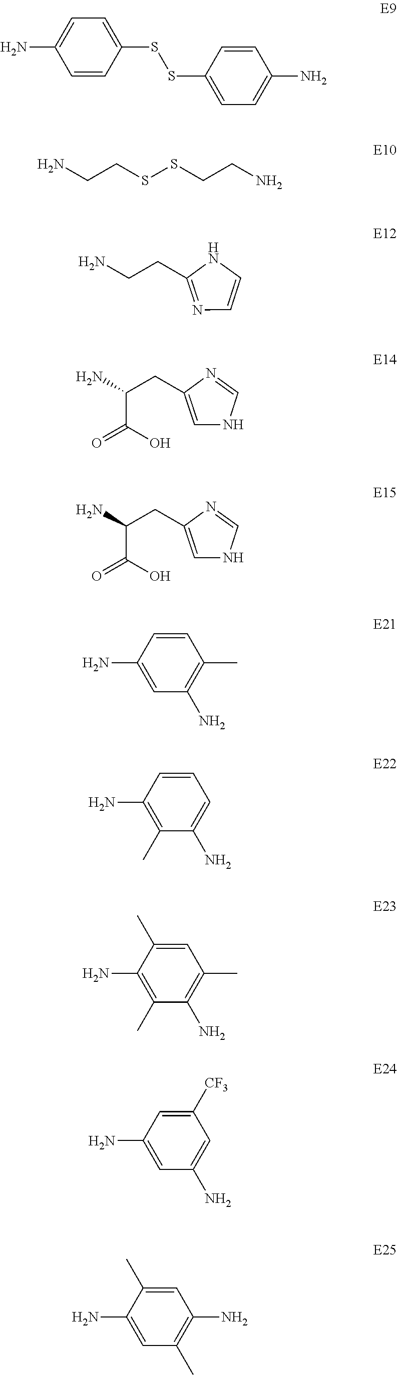

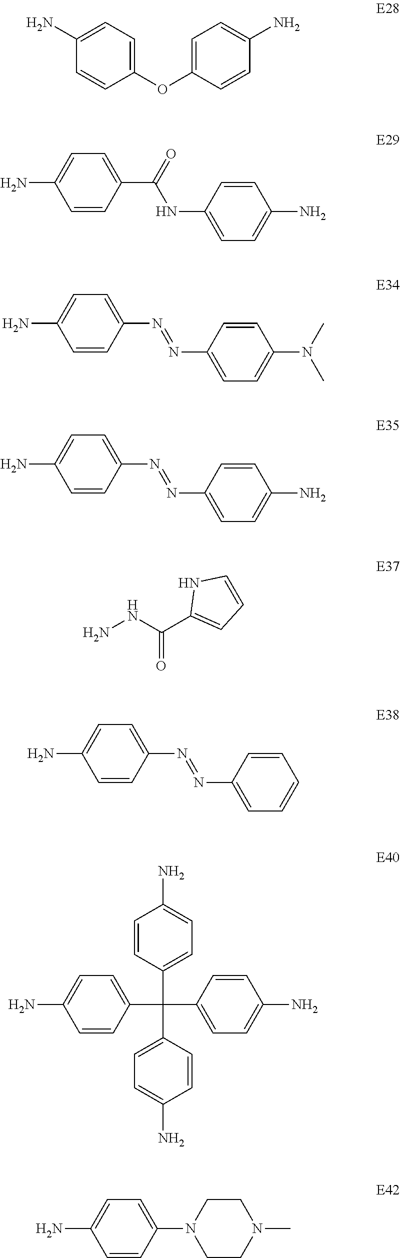

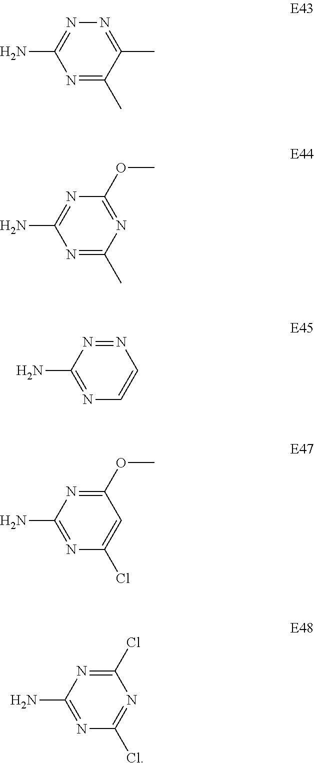

8. The nanoparticle of any one of claims 4-7, wherein the amine-terminated bioreducible poly(beta-amino ester) is selected from the group consisting of R646 and R647: 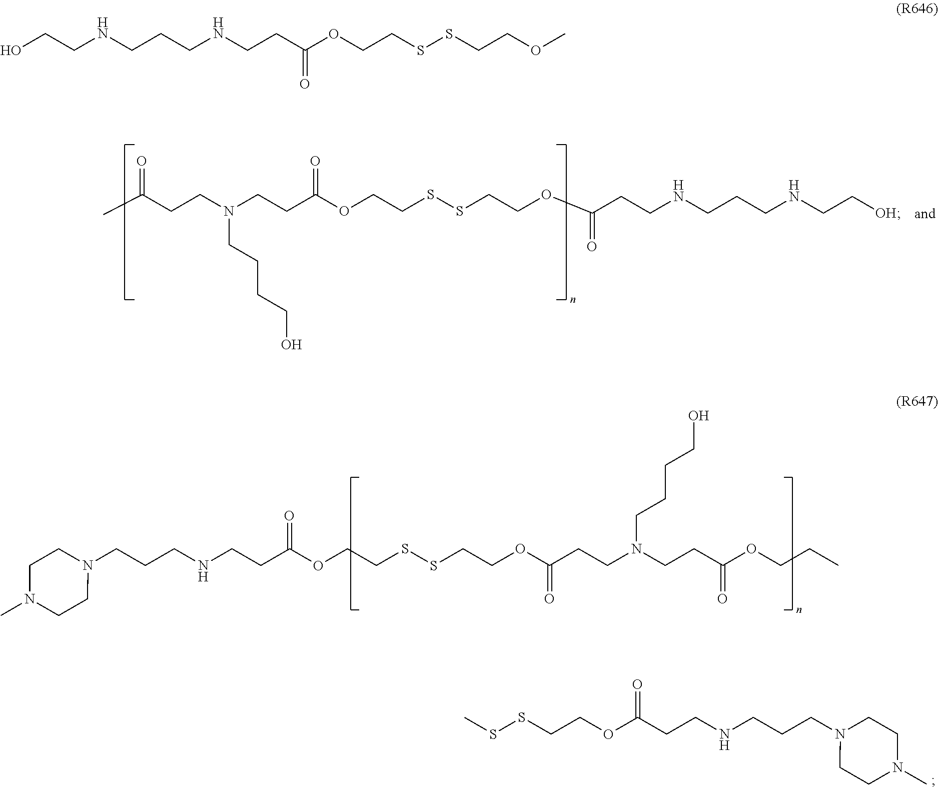
wherein n is an integer from 1 to 10,000.

9. The nanoparticle of claim 1, wherein the crosslinking polymer comprises an acrylate-terminated bioreducible poly(beta-amino ester) having at least one disulfide bond.

10. The nanoparticle of claim 9, wherein the acrylate-terminated bioreducible poly(beta-amino ester) comprises a monomer having the following structure:

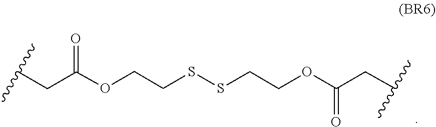 [SMILES: *CC(=O)OCCSSCCOC(=O)C*]

11. The nanoparticle of claim 10, wherein the acrylate-terminated bioreducible poly(beta-amino ester) comprises a compound having the following structure: 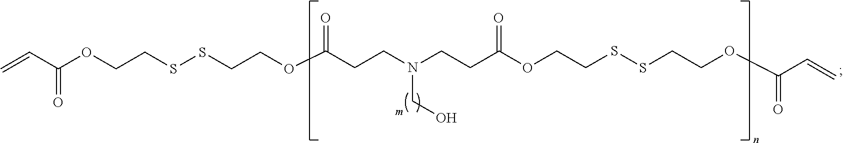
wherein:
n is an integer from 1 to 10,000; and
m is an integer selected from the group consisting of 2, 3, 4, 5, 6, 7, 8, and 9.

12. The nanoparticle of claim 11, wherein the acrylate-terminated bioreducible poly(beta-amino ester) comprises R64-Ac: 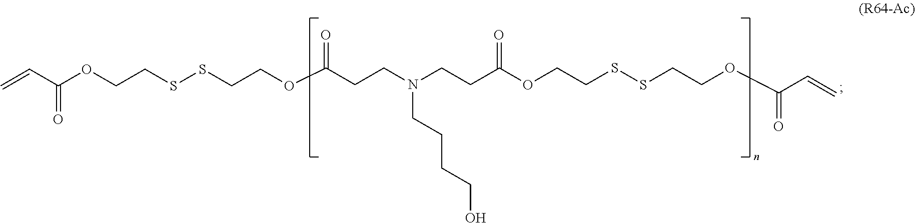
wherein n is an integer from 1 to 10,000.

13. The nanoparticle of claim 1, wherein the crosslinking polymer and the cationic polymer have a mass ratio (weight ratio) of about 1:1 to 1:5 (w/w).

14. The nanoparticle of claim 1, wherein the crosslinking polymer and the cationic polymer have a mass ratio (weight ratio) of about 1:3 (w/w).

15. The nanoparticle of claim 1, wherein the one or more nucleic acids are selected from the group consisting of DNA, RNA, siRNA, mRNA, miRNA, isRNA, agRNA, and smRNA.

16. The nanoparticle of claim 15, wherein the one or more nucleic acids comprises siRNA.

17. The nanoparticle of any one of claims 1-16, wherein the nanoparticle has a polymer:nucleic acid mass ratio (weight/weight or w/w) ranging from about 20 (w/w) polymer:nucleic acid to about 2,000 (w/w) polymer:nucleic acid.

18. The nanoparticle of any one of claims 1-17, wherein the nanoparticle has a substantially neutral surface charge.

19. The nanoparticle of any one of claims 1-18, wherein the nanoparticle has a particle size ranging from about 50 nm to 500 nm.

20. The nanoparticle of any one of claims 1-19, wherein the nanoparticle has a particle size ranging from about 100 nm to 250 nm.

21. A pharmaceutical formulation comprising a nanoparticle of any one of claims 1-20 and a pharmaceutically acceptable carrier.

22. A method for preparing a crosslinked nanoparticle, the method comprising:
(a) mixing a bioreducible cationic polymer, and crosslinking polymer, and one or more nucleic acids to form a self-assembled bioreducible nanoparticle;
(b) incubating the self-assembled bioreducible nanoparticle for a period of time;
(c) adding a photoinitiator to the self-assembled bioreducible nanoparticle; and
(d) exposing the self-assembled bioreducible nanoparticle and photoinitiator to ultraviolet light to form a crosslinked bioreducible nanoparticle comprising the one or more nucleic acids.

23. The method of claim 22, wherein the cationic polymer comprises a bioreducible poly(beta-amino ester).

24. The method of claim 23, wherein the bioreducible poly(beta-amino ester) comprises at least one degradable ester bond and at least one disulfide bond.

25. The method of claim 23 or claim 24, wherein the bioreducible poly(beta-amino ester) comprises an amine-terminated bioreducible poly(beta-amino ester).

26. The method of claim 25, further comprising synthesizing the amine-terminated bioreducible poly(beta-amino ester) with a BR6 monomer:

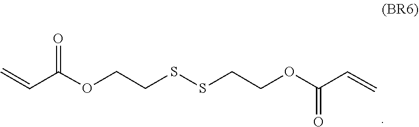 [SMILES: C=CC(=O)OCCSSCCOC(=O)C=C]

27. The method of claim 26, further comprising synthesizing the amine-terminated bioreducible poly(beta-amino ester) with a BR6 monomer and a linear amino alcohol monomer of the general formula NH₂—R—OH, where R comprises an alkyl chain consisting of 2, 3, 4, 5, 6, 7, 8, or 9 carbons.

28. The method of claim 27, wherein the amine-terminated bioreducible poly(beta-amino ester) comprises a compound having the following structure: 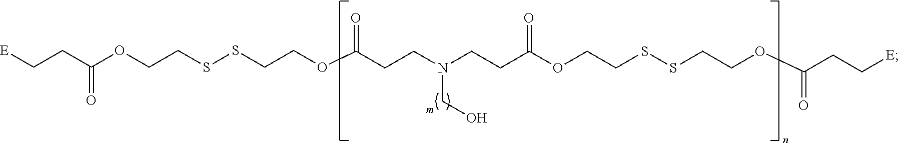
wherein:
n is an integer from 1 to 10,000;
m is an integer selected from the group consisting of 2, 3, 4, 5, 6, 7, 8, and 9; and
E is an amine-containing endcapping group.

29. The method of claim 28, wherein the amine-containing endcapping group is selected from the group consisting of:

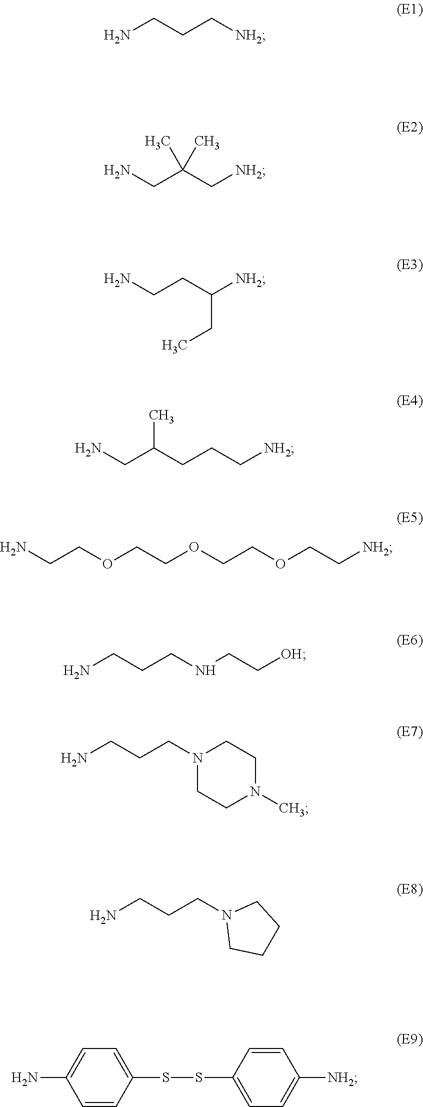

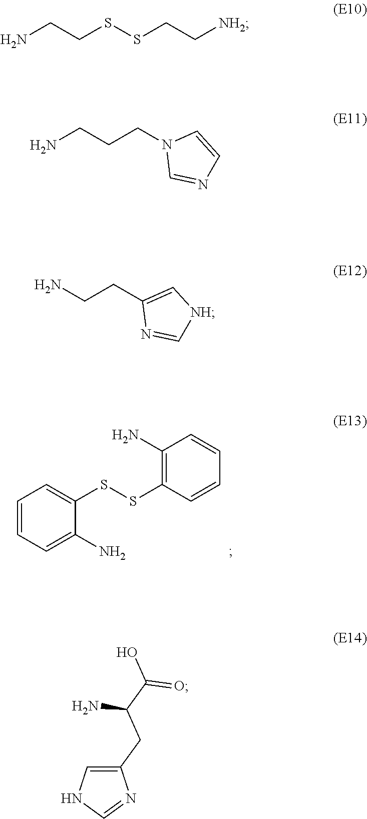 [SMILES: NCCSSCCN ; NCCCn1ccnc1 ; NCCc1c[nH]cn1 ; Nc1ccccc1SSc1ccccc1N ; N[C@H](Cc1c[nH]cn1)C(=O)O]

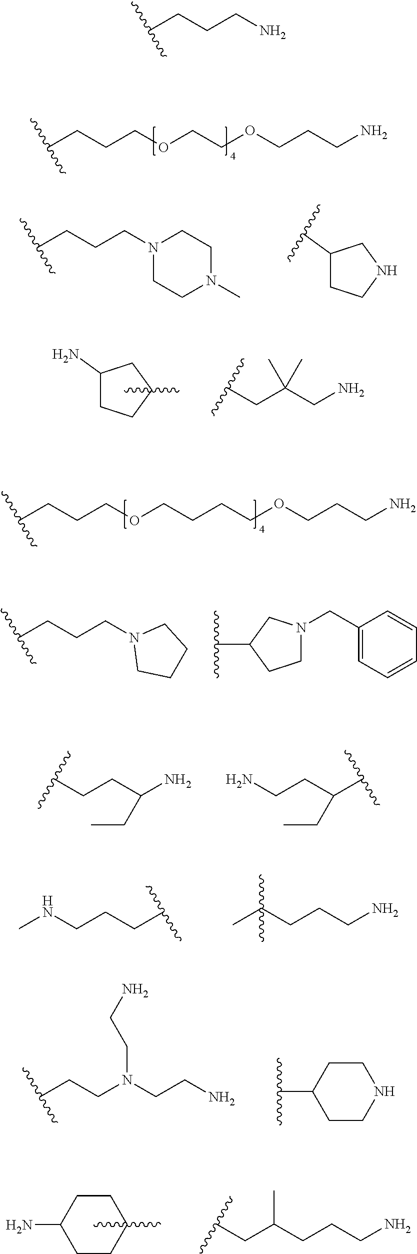

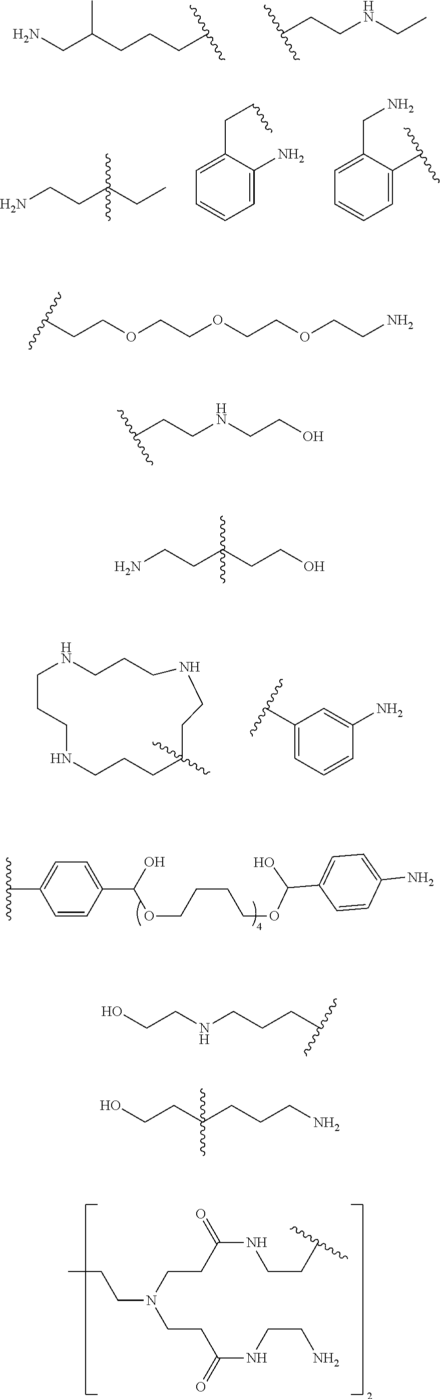

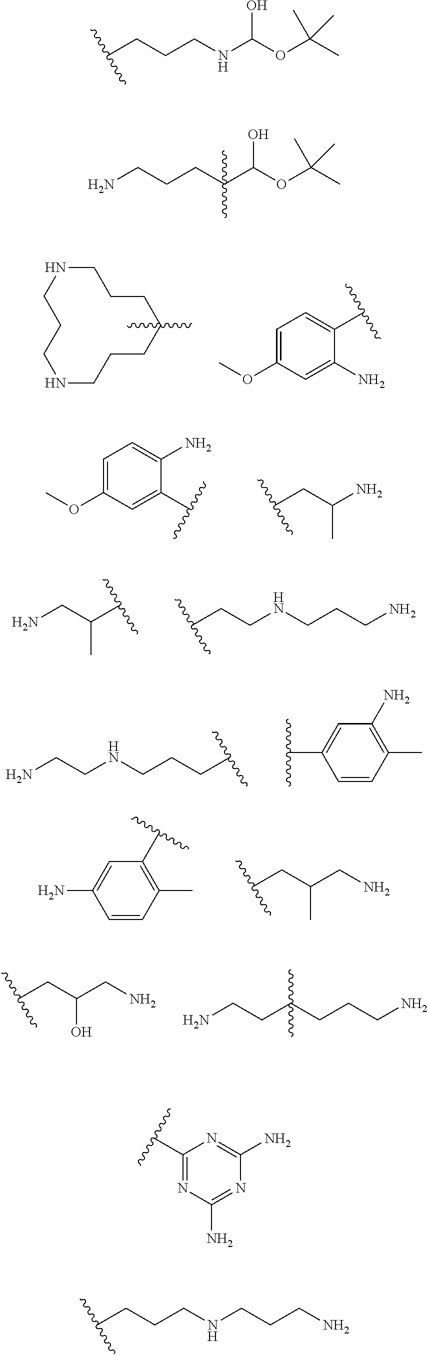

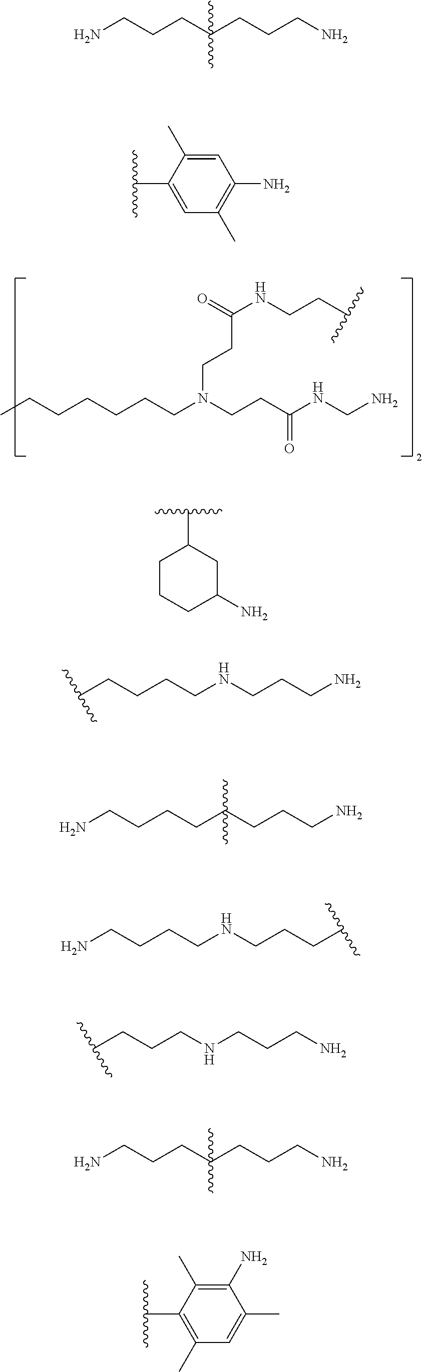

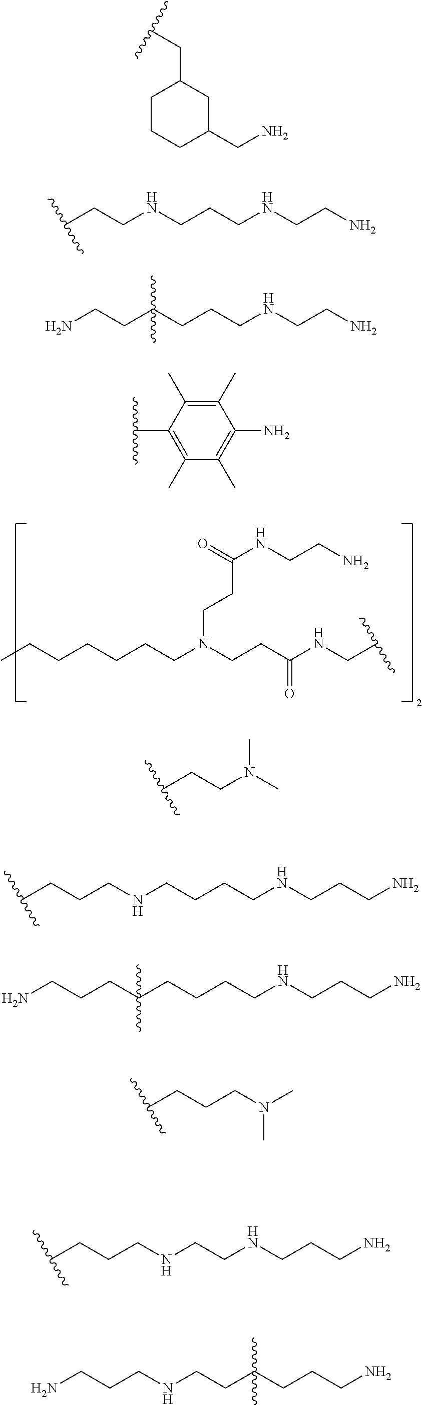

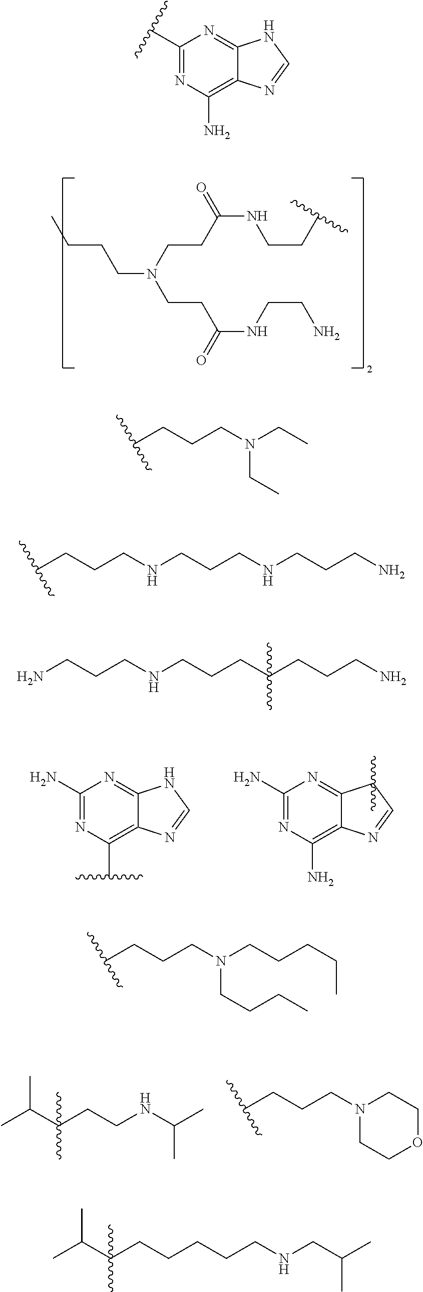

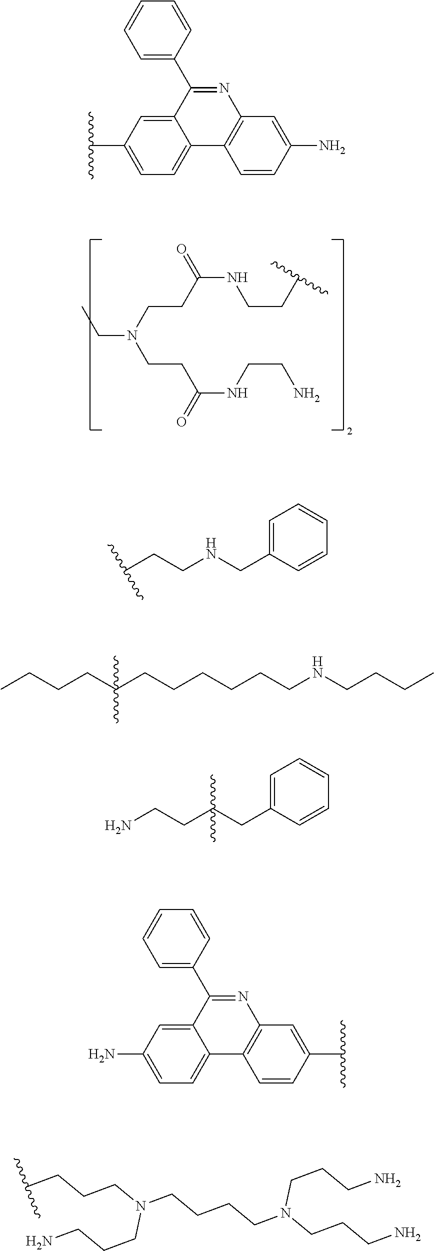

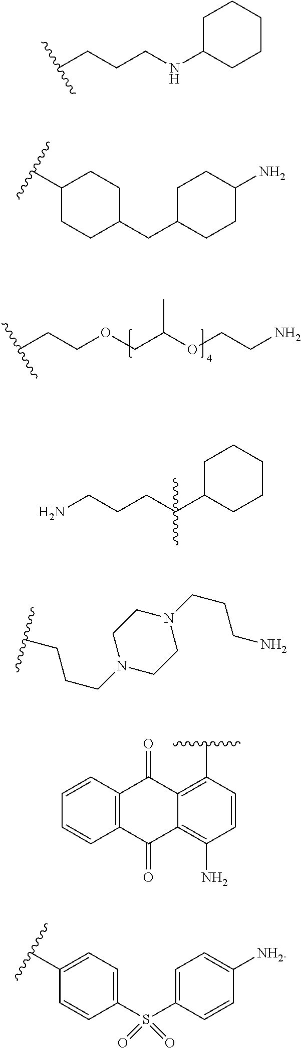

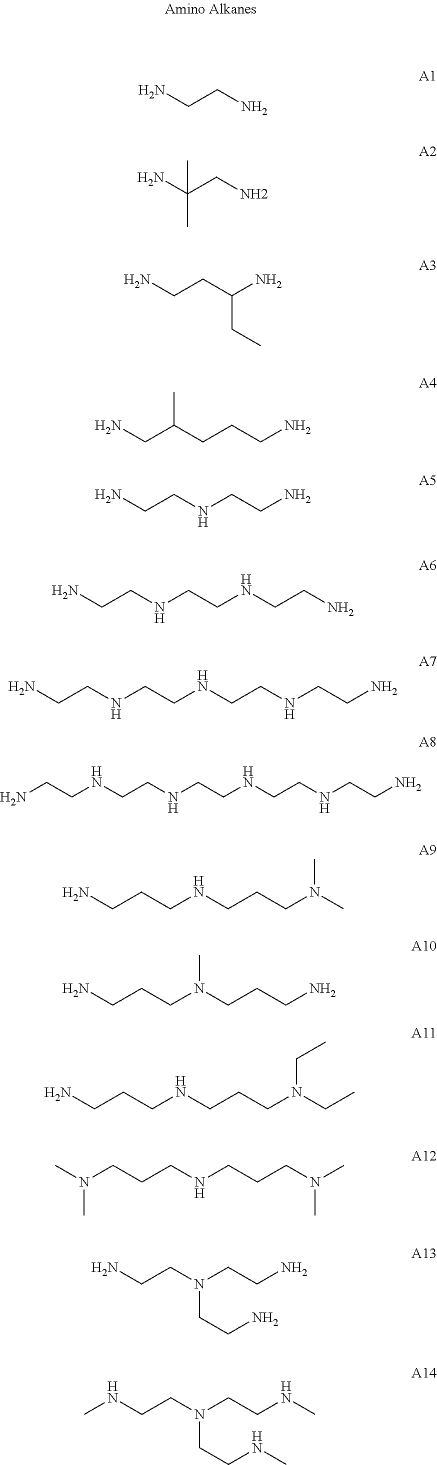

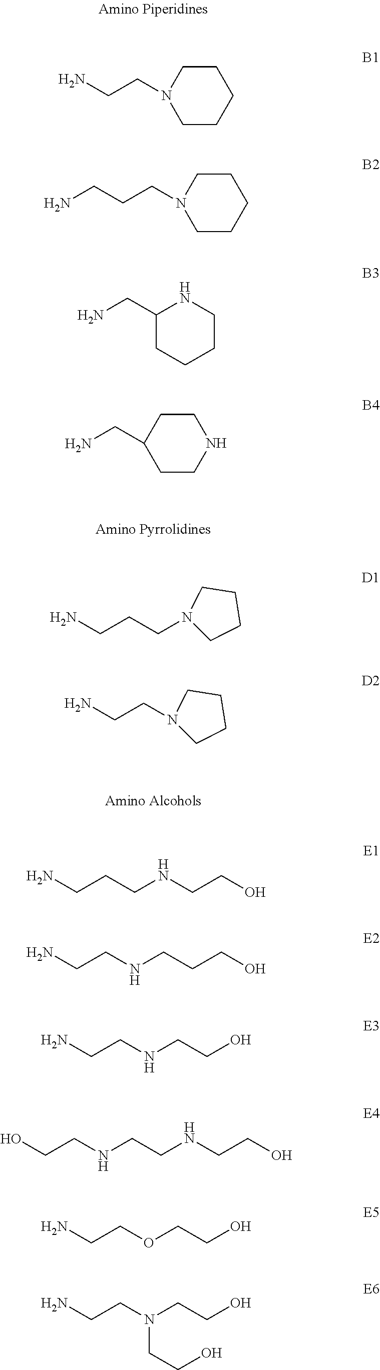

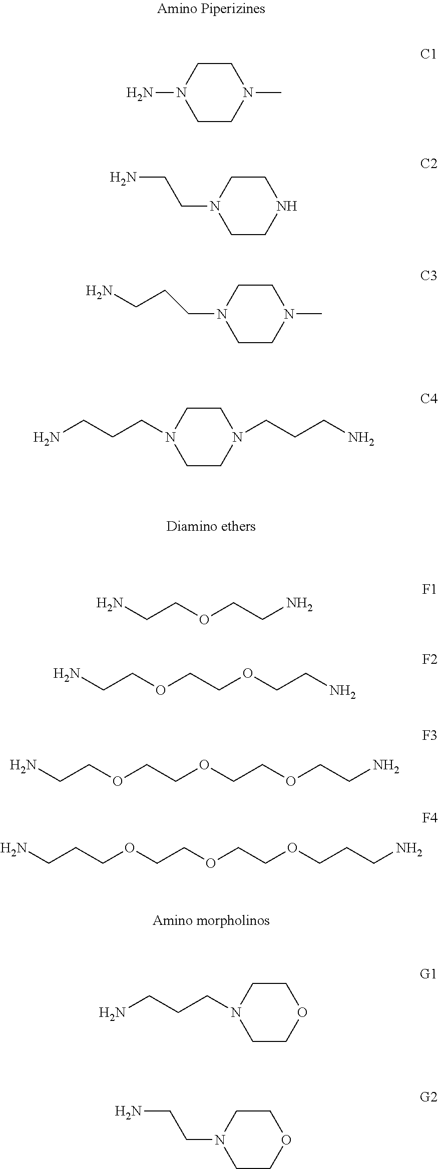

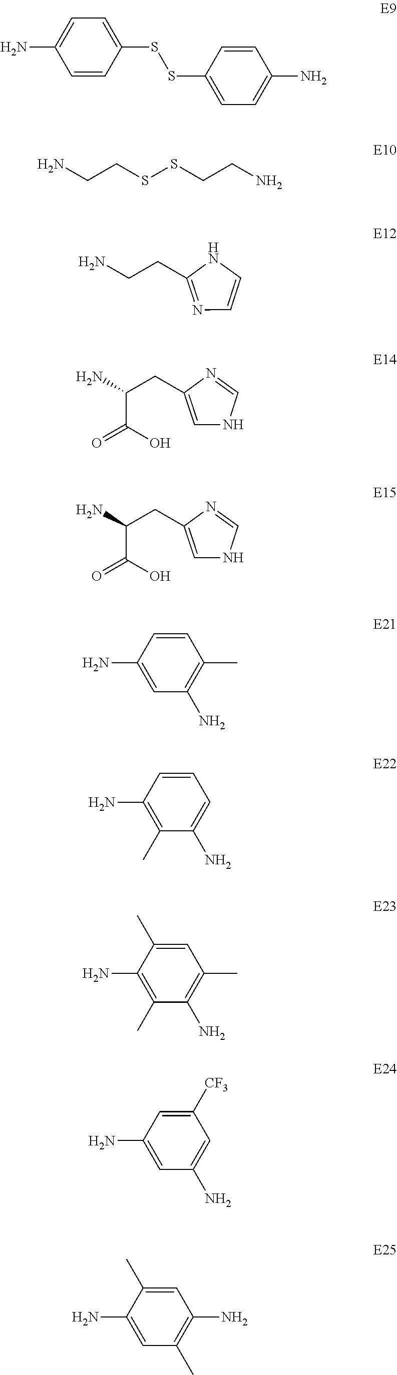

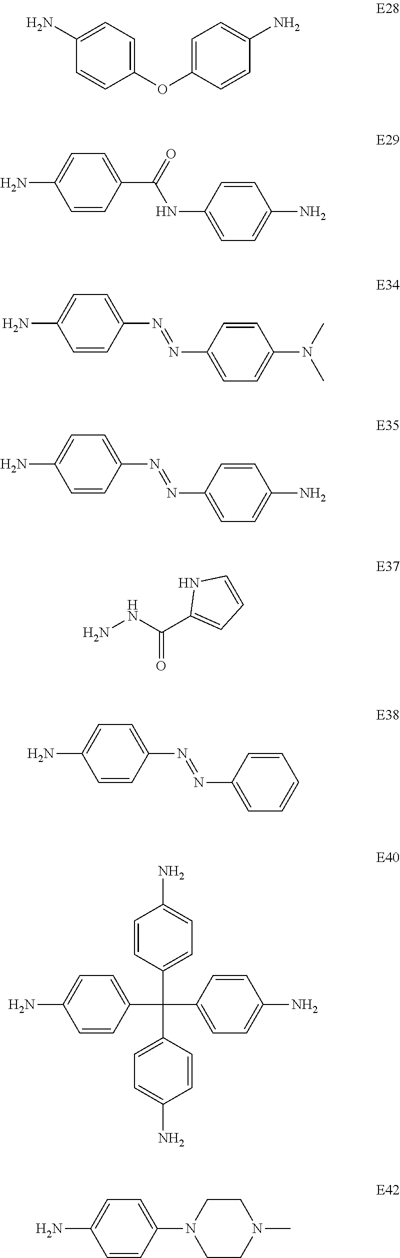

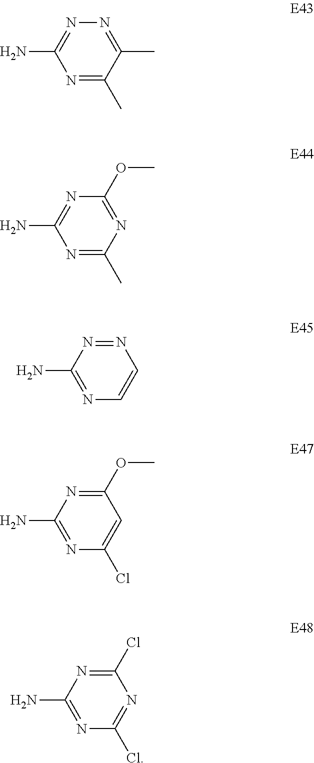

30. The method of any one of claims 25-29, wherein the amine-terminated bioreducible poly(b-amino ester) is selected from the group consisting of R646 and R647: 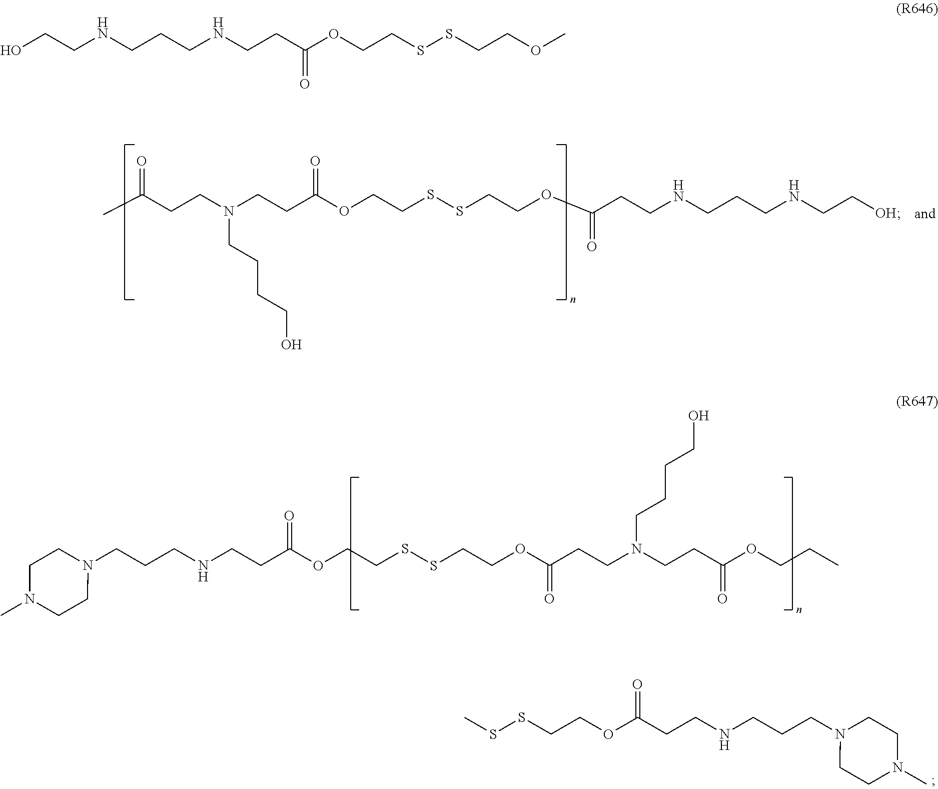
wherein n is an integer from 1 to 10,000.

31. The method of claim 22, wherein the crosslinking polymer comprises an acrylate-terminated bioreducible poly(beta-amino ester) having at least one disulfide bond.

32. The method of claim 31, further comprising synthesizing the acrylate-terminated bioreducible poly(beta-amino ester) with a BR6 monomer:

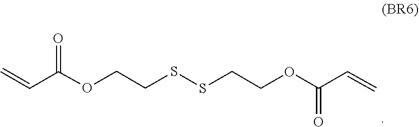 [SMILES: C=CC(=O)OCCSSCCOC(=O)C=C]

33. The method of claim 32, wherein the acrylate-terminated bioreducible poly(beta-amino ester) comprises a compound having the following structure: 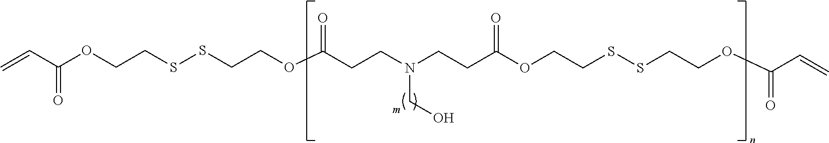
wherein:
n is an integer from 1 to 10,000; and
m is an integer selected from the group consisting of 2, 3, 4, 5, 6, 7, 8, and 9.

34. The method of claim 32, wherein the acrylate-terminated bioreducible poly(beta-amino ester) comprises R64-Ac: 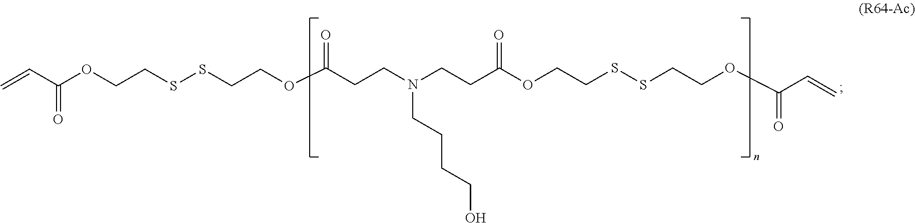
wherein n is an integer from 1 to 10,000.

35. The method of claim 22, wherein the crosslinking polymer and the cationic polymer have a mass ratio (weight ratio) of about 1:1 to 1:5 (w/w).

36. The method of claim 35, wherein the crosslinking polymer and the cationic polymer have a mass ratio (weight ratio) of about 1:3 (w/w).

37. The method of claim 22 or 23, wherein the one or more nucleic acids are selected from the group consisting of DNA, RNA, siRNA, mRNA, miRNA, isRNA, agRNA, and smRNA.

38. The method of claim 37, wherein the nucleic acid comprises siRNA.

39. The method of any one of claims 22-38, wherein the nanoparticle has a polymer:nucleic acid mass ratio (weight/weight or w/w) ranging from about 20 (w/w) polymer:nucleic acid to about 2,000 (w/w) polymer:nucleic acid.

40. A method for treating a cancer, the method comprising administering to a subject in need of treatment thereof a therapeutically effective amount of a nanoparticle of any one of claims 1-20 or the pharmaceutical formulation of claim 21 to treat the cancer.

41. The method of claim 40, wherein the nanoparticle enters a cytosol of a cell of the subject.

42. The method of claim 40, wherein the at least one disulfide bond of the bioreducible poly(beta-amino ester) is reductively degraded in the cytosol to release the nucleic acid in the cytosol of the cell of the subject.

43. The method of claim 40, wherein the at least one disulfide bond is reductively degraded via glutathione.

44. The method of claim 40, comprising systemically administering the nanoparticle or pharmaceutical formulation.

45. The method of claim 40, wherein the one or more nanoparticles are delivered to one or more organs beyond the liver.

46. The method of claim 45, wherein the one or more organs beyond the liver comprises the lungs.

47. The method of claim 45, comprising preferential uptake of the nucleic acid in one or more cancer cells.

48. The method of claim 40, wherein the crosslinked nanoparticle exhibits enhanced colloidal stability in the bloodstream compared to a non-crosslinked nanoparticle.

49. The method of claim 40, wherein the crosslinked nanoparticle exhibits reduced adsorption of anionic serum proteins compared to a non-crosslinked nanoparticle.

50. The method of any one of claims 40-49, wherein the cancer is selected from the group consisting of brain cancer, melanoma, lung cancer, breast cancer, prostate cancer, and colorectal cancer.

51. The method of claim 50, wherein the brain cancer comprises a glioblastoma.
